(19) United States
(12) Patent Application Publication
(10) Pub. No.: US 2023/0162004 A1
(43) Pub. Date: May 25, 2023

(54) DEEP NEURAL NETWORKS FOR ESTIMATING POLYGENIC RISK SCORES

(71) Applicant: THE BOARD OF REGENTS OF THE UNIVERSITY OF OKLAHOMA, Norman, OK (US)

(72) Inventors: Chongle PAN, Norman, OK (US); Adrien BADRÉ, Norman, OK (US)

(73) Assignee: THE BOARD OF REGENTS OF THE UNIVERSITY OF OKLAHOMA, Norman, OK (US)

(21) Appl. No.: 17/930,505

(22) Filed: Sep. 8, 2022

Related U.S. Application Data
(60) Provisional application No. 63/241,645, filed on Sep. 8, 2021.

(51) Int. Cl. G06N 3/04; G06N 3/02

(52) U.S. Cl. CPC G06N 3/0454; G06N 3/02

(57) ABSTRACT

Disclosed herein are systems, methods, devices, and media for the risk for diseases and conditions in a patient. Deep neural networks enable the automated analysis of a patient’s SNP profile to generate predictions of a patient’s risk for developing a disease or condition.

### REFERENCE TO RELATED APPLICATIONS

The present application claims the priority benefit of U.S. Provisional Application No. 63/241,645, filed Sep. 8, 2021, the entire contents of which is incorporated herein by reference.

### BACKGROUND

Breast cancer is the second deadliest cancer for U.S. women. Approximately one in eight women in the U.S. will develop invasive breast cancer over the course of their lifetime (NIH, 2019). Early detection of breast cancer is an effective strategy to reduce the death rate. If breast cancer is detected in the localized stage, the 5-year survival rate is 99% (NIH, 2019). However, only ∼62% of the breast cancer cases are detected in the localized stage (NIH, 2019). In ∼30% of the cases, breast cancer is detected after it spreads to the regional lymph nodes, reducing the 5-year survival rate to 85%. Furthermore, in 6% of cases, the cancer is diagnosed after it has spread to a distant part of the body beyond the lymph nodes and the 5-year survival rate is reduced to 27%. To detect breast cancer early, the US Preventive Services Task Force (USPSTF) recommends a biennial screening mammography for women over 50 years old. For women under 50 years old, the decision for screening must be individualized to balance the benefit of potential early detection against the risk of false positive diagnosis. False-positive mammography results, which typically lead to unnecessary follow-up diagnostic testing, become increasingly common for women 40 to 49 years old (Nelson et al., 2009). Nevertheless, for women with high risk for breast cancer (i.e. a lifetime risk of breast cancer higher than 20%), the American Cancer Society advises a yearly breast MRI and mammogram starting at 30 years of age (Oeffinger et al., 2015).

Polygenic risk scores (PRS) assess the genetic risks of complex diseases based on the aggregate statistical correlation of a disease outcome with many genetic variations over the whole genome. Single-nucleotide polymorphisms (SNPs) are the most commonly used genetic variations. While genome-wide association studies (GWAS) report only SNPs with statistically significant associations to phenotypes (Dudbridge, 2013), PRS can be estimated using a greater number of SNPs with higher adjusted p-value thresholds to improve prediction accuracy.

Previous research has developed a variety of PRS estimation models based on Best Linear Unbiased Prediction (BLUP), including gBLUP (Clark et al., 2013), rr-BLUP (Whittaker et al., 2000; Meuwissen et al., 2001), and other derivatives (Maier et al., 2015; Speed & balding, 2014). These linear mixed models consider genetic variations as fixed effects and use random effects to account for environmental factors and individual variability. Furthermore, linkage disequilibrium was utilized as a basis for the LDpred (Vilhjalmsson et al., 2015; Khera et al., 2018) and PRS-CS (Ge et al., 2019) algorithms.

PRS estimation can also be defined as a supervised classification problem. The input features are genetic variations and the output response is the disease outcome. Thus, machine learning techniques can be used to estimate PRS based on the classification scores achieved (Ho et al., 2019). A large-scale GWAS dataset may provide tens of thousands of individuals as training examples for model development and benchmarking. Wei et al. (2019) compared support vector machine and logistic regression to estimate PRS of Type-1 diabetes. The best Area Under the receiver operating characteristic Curve (AUC) was 84% in this study. More recently, neural networks have been used to estimate human height from the GWAS data, and the best R² scores were in the range of 0.4 to 0.5 (Bellot et al., 2018). Amyotrophic lateral sclerosis was also investigated using Convolutional Neural Networks (CNN) with 4511 cases and 6127 controls (Yin et al., 2019) and the highest accuracy was 76.9%.

Significant progress has been made for estimating PRS for breast cancer from a variety of populations. In a recent study (Mavaddat et al., 2019), multiple large European female cohorts were combined to compare a series of PRS models. The most predictive model in that study used lasso regression with 3,820 SNPs and obtained an AUC of 65%. A PRS algorithm based on the sum of log odds ratios of important SNPs for breast cancer was used in the Singapore Chinese Health Study (Chan et al., 2018) with 46 SNPs and 56.6% AUC, the Shanghai Genome-Wide Association Studies (Wen et al., 2016) with 44 SNPs and 60.6% AUC, and a Taiwanese cohort (Hsieh et al., 2017) with 6 SNPs and 59.8% AUC. A pruning and thresholding method using 5,218 SNPs reached an AUC of 69% for the UK Biobank dataset (Khera et al., 2018).

### BRIEF DESCRIPTION OF THE DRAWINGS

The following drawings form part of the present specification and are included to further demonstrate certain aspects of the present disclosure. The accompanying drawings illustrate one or more implementations described herein and, together with the description, explain these implementations. The drawings are not intended to be drawn to scale, and certain features and certain views of the figures may be shown exaggerated, to scale or in schematic in the interest of clarity and conciseness. Not every component may be labeled in every drawing. Like reference numerals in the figures may represent and refer to the same or similar element or function.

FIG. 1. Computational workflow of predictive genomics. The DRIVE dataset was randomly split into the training set, the validation set, and the test set. Only the training set was used for association analysis, which generated the p-values for selection of SNPs as input features. The training data was then used to train machine learning models and statistical models. The validation set was used to select the best hyperparameters for each model based on the validation AUC score. Finally, the test set was used for performance benchmarking and model interpretati on.

FIGS. 2A-2B. SNP filtering and model training for DNN. (FIG. 2A) Manhattan plot from the association analysis. Each point represents a SNP with its p-value in the log10 scale on the y-axis and its position in a chromosome on the x-axis. The x-axis is labeled with the chromosome numbers. Chromosome 23 represents the X chromosome. Chromosomes 24 and 25 represent the pseudoautosomal region and non-pseudoautosomal region of the Y chromosome, respectively. Chromosome 26 designates mitochondrial chromosome. The top horizontal line marks the p-value cutoff at 9.5x10⁻⁸ and the bottom horizontal line marks the p-value cutoff at 10^{~3}. (FIG. 2B) Performance of the DNN models trained using five SNP sets filtered with increasing p-value cutoffs. The models were compared by their training costs and performances in the training and validation sets.

FIG. 3. Comparison of machine learning approaches for PRS estimation. The performance of the models were represented as Receiver Operating Characteristic (ROC) curves. At the X-axis value of 0.4, the top solid line represents “DNN” and the bottom solid line represents “Decision Tree”. The Area under the ROC curve (AUC) and the accuracy from the test set are shown in the legend. The DNN model outperformed the other machine learning models in terms of AUC and accuracy.

FIG. 4. Score histograms of DNN, BLUP, BayesA and LDpred. The case and control populations are shown in the right-shifted and left-shifted histograms, respectively. The vertical line represents the score cutoff corresponding to the precision of 90% for each model. DNN had a much higher recall than the other algorithms at the 90% precision.

FIG. 5. Effects of dropout and batch normalization on the 5,273-SNP DNN model. At the X-axis value of 100, the lines represent, from top to bottom, “DNN with dropout and batch normalization”, “DNN with dropout and without batch normalization”, “DNN without dropout and without batch normalization”, and “DNN without dropout and with batch normalization”.

FIG. 6. Venn diagram of important SNPs found by LIME, DeepLift, and association analysis. The top left circle represents the top-100 salient SNPs identified by LIME. The top right circle represents the top-100 salient SNPs identified by DeepLift. The large circle represents the 1,061 SNPs that had p-values lower than the Bonferroni-corrected critical value. The numbers in the Venn diagram show the sizes of the intersections and complements among the three sets of SNPs.

FIGS. 7A-7B. Genotype-phenotype relationships for salient SNPs used in the DNN model. For each Genotype value, the left bar represents “Case” and the right bar represents “Control”. (FIG. 7A) Four salient SNPs with linear relationships as shown by the lines and the significant association p-values. (FIG. 7B) Four salient SNPs with non-linear relationships as shown by the lines and the insignificant association p-values. The DNN model was able to use SNPs with non-linear relationships as salient features for prediction.

FIG. 8. First-order model-wise interpretation. The three bars of a feature represent the FP, DP, and IP scores, from left to right, of this feature in the LINA model.

FIG. 9. Second-order model-wise interpretation. The second-order model-wise importance scores (SP) are undirected between two features and are shown in a symmetric matrix as a heatmap. The importance scores for the feature self-interactions are set to zero in the diagonal of the matrix.

FIG. 10. An example LINA model for structured data. The LINA model uses an input layer and multiple hidden layers to output the attention weights in the attention layer. The attention weights are then multiplied with the input features element-wise in the linearization layer and then with the coefficients in the output layer. The crossed neurons in the linearization layer represent element-wise multiplication of their two inputs. The incoming connections to the crossed neurons have a constant weight of 1.

### DETAILED DESCRIPTION

The present disclosure relates generally to the field of deep learned-based medical diagnostics. More particularly, it concerns deep neural networks and methods for training deep neural networks to provide estimated polygenic risk scores.

In one embodiment the present disclosure is directed to computer-implemented methods of training a deep neural network for estimating a polygenic risk score for a disease. In some aspects, the method comprise collecting a first set of SNPs from at least 1,000 subjects with a known disease outcome from a database and a second set of SNPs from at least 1,000 other subjects with a known disease outcome from a database; encoding, independently, the first set of SNPs and the second set of SNPs by: labeling each subject as either a disease case or a control case based on the known disease outcome for the subject, and labeled each SNP in each subject as either homozygous with minor allele, heterozygous allele, or homozygous with the dominant allele; optionally applying one or more filter to the first encoded set to create a first modified set of SNPs; training the deep neural network using the first encoded set of SNPs or the first modified set of SNPs; and validating the deep neural network using the second encoded set of SNPs.

In some aspects, the filter comprises a p-value threshold.

In some aspects, the first set of SNPs and the second set of SNPs are both from at least 10,000 subjects. In some aspects, the SNPs are genome-wide. In some aspects, the SNPs are representative of at least 22 chromosomes. In some aspects, both the first set of SNPs and the second set of SNPs comprise the same at least 2,000 SNPs.

In some aspects, the disease is cancer. In some aspects, the cancer is breast cancer. In some aspects, the SNPs include at least five of the SNPs listed in Table 2.

In some aspects, the trained deep neural network has an accuracy of at least 60%. In some aspects, the trained deep neural network has an AUC of at least 65%.

In some aspects, the trained deep neural network comprises at least three hidden layers, and each layer comprises multiple neurons. For example, each layer may comprise 1000, 250, or 50 neurons.

In some aspects, the training the deep neural network comprises using stochastic gradient descent with regularization, such as dropout.

In some aspects, the deep neural network comprises a linearization layer on top of a deep inner attention neural network. In some aspects, the linearization layer computes an output as an element-wise multiplication product of input features, attention weights, and coefficients. In some aspects, the network learns a linear function of an input feature vector, coefficient vector, and attention vector. In some aspects, the attention vector is computed from the input feature vector using a multi-layer neural network. In some aspects, all hidden layers of the multi-layer neural network use a non-linear activation function, and wherein the attention layer uses a linear activation function. In some aspects, the layers of the inner attention neural network comprise 1000, 250, or 50 neurons before the attention layer.

In one embodiment, provided herein are methods of using a deep neural network training using data from subjects with a disease by the methods of the present embodiments to estimate a polygenic risk score for a patient for the disease. In some aspects, the methods comprise collecting a set of SNPs from a subject with an unknown disease outcome; encoding the set of SNPs by labeled each SNP in the subject as either homozygous with minor allele, heterozygous allele, or homozygous with the dominant allele; and applying the deep neural network to obtain an estimated polygenic risk score for the patient for the disease.

In some aspects, the methods further comprise performing, or having performed, further screening for the disease if the polygenic risk score indicates that the patient is at risk for the disease.

In one embodiment, provided herein are methods for determining a polygenic risk score for a disease for a subject. In some aspects, the methods comprise (a) obtaining a plurality of SNPs from genome of the subject; (b) generating a data input from the plurality of SNPs; and (c) determining the polygenic risk score for the disease by applying to the data input a deep neural network trained by the methods of the present embodiments. In some aspects, the methods further comprise performing, or having performed, further screening for the disease if the polygenic risk score indicates that the patient is at risk for the disease. In some aspects, the disease is breast cancer, and wherein the method comprises performing, or having performed, yearly breast MRI and mammogram if the patient’s polygenic risk score is greater than 20%.

In one embodiment, provided herein are polygenic risk score classifiers comprising a deep neural network that has been trained according to the methods provided herein.

In one non-limiting embodiment, the present disclosure is directed to a deep neural network (DNN) that was tested for breast cancer PRS estimation using a large cohort containing 26,053 cases and 23,058 controls. The performance of the DNN was shown to be higher than alternative machine learning algorithms and other statistical methods in this large cohort. Furthermore, DeepLift (Shrikumar et al., 2019) and LIME (Ribeiro et al., 2016) were used to identify salient SNPs used by the DNN for prediction.

Before further describing various embodiments of the apparatus, component parts, and methods of the present disclosure in more detail by way of exemplary description, examples, and results, it is to be understood that the embodiments of the present disclosure are not limited in application to the details of apparatus, component parts, and methods as set forth in the following description. The embodiments of the apparatus, component parts, and methods of the present disclosure are capable of being practiced or carried out in various ways not explicitly described herein. As such, the language used herein is intended to be given the broadest possible scope and meaning; and the embodiments are meant to be exemplary, not exhaustive. Also, it is to be understood that the phraseology and terminology employed herein is for the purpose of description and should not be regarded as limiting unless otherwise indicated as so. Moreover, in the following detailed description, numerous specific details are set forth in order to provide a more thorough understanding of the disclosure. However, it will be apparent to a person having ordinary skill in the art that the embodiments of the present disclosure may be practiced without these specific details. In other instances, features which are well known to persons of ordinary skill in the art have not been described in detail to avoid unnecessary complication of the description. While the apparatus, component parts, and methods of the present disclosure have been described in terms of particular embodiments, it will be apparent to those of skill in the art that variations may be applied to the apparatus, component parts, and/or methods and in the steps or in the sequence of steps of the method described herein without departing from the concept, spirit, and scope of the inventive concepts as described herein. All such similar substitutes and modifications apparent to those having ordinary skill in the art are deemed to be within the spirit and scope of the inventive concepts as disclosed herein.

All patents, published patent applications, and non-patent publications referenced or mentioned in any portion of the present specification are indicative of the level of skill of those skilled in the art to which the present disclosure pertains, and are hereby expressly incorporated by reference in their entirety to the same extent as if the contents of each individual patent or publication was specifically and individually incorporated herein.

Unless otherwise defined herein, scientific and technical terms used in connection with the present disclosure shall have the meanings that are commonly understood by those having ordinary skill in the art. Further, unless otherwise required by context, singular terms shall include pluralities and plural terms shall include the singular.

As utilized in accordance with the methods and compositions of the present disclosure, the following terms and phrases, unless otherwise indicated, shall be understood to have the following meanings: The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or when the alternatives are mutually exclusive, although the disclosure supports a definition that refers to only alternatives and “and/or.” The use of the term “at least one” will be understood to include one as well as any quantity more than one, including but not limited to, 2, 3, 4, 5, 6, 7, 8, 9, 10, 15, 20, 30, 40, 50, 100, or any integer inclusive therein. The phrase “at least one” may extend up to 100 or 1000 or more, depending on the term to which it is attached; in addition, the quantities of 100/1000 are not to be considered limiting, as higher limits may also produce satisfactory results. In addition, the use of the term “at least one of X, Y and Z” will be understood to include X alone, Y alone, and Z alone, as well as any combination of X, Y and Z.

As used in this specification and claims, the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

Throughout this application, the terms “about” or “approximately” are used to indicate that a value includes the inherent variation of error for the apparatus, composition, or the methods or the variation that exists among the objects, or study subjects. As used herein the qualifiers “about” or “approximately” are intended to include not only the exact value, amount, degree, orientation, or other qualified characteristic or value, but are intended to include some slight variations due to measuring error, manufacturing tolerances, stress exerted on various parts or components, observer error, wear and tear, and combinations thereof, for example.

The terms “about” or “approximately”, where used herein when referring to a measurable value such as an amount, percentage, temporal duration, and the like, is meant to encompass, for example, variations of ± 20% or ± 10%, or ± 5%, or ± 1%, or ± 0.1% from the specified value, as such variations are appropriate to perform the disclosed methods and as understood by persons having ordinary skill in the art. As used herein, the term “substantially” means that the subsequently described event or circumstance completely occurs or that the subsequently described event or circumstance occurs to a great extent or degree. For example, the term “substantially” means that the subsequently described event or circumstance occurs at least 90% of the time, or at least 95% of the time, or at least 98% of the time.

As used herein any reference to “one embodiment” or “an embodiment” means that a particular element, feature, structure, or characteristic described in connection with the embodiment is included in at least one embodiment. The appearances of the phrase “in one embodiment” in various places in the specification are not necessarily all referring to the same embodiment.

As used herein, all numerical values or ranges include fractions of the values and integers within such ranges and fractions of the integers within such ranges unless the context clearly indicates otherwise. A range is intended to include any sub-range therein, although that sub-range may not be explicitly designated herein. Thus, to illustrate, reference to a numerical range, such as 1-10 includes 1, 2, 3, 4, 5, 6, 7, 8, 9, 10, as well as 1.1, 1.2, 1.3, 1.4, 1.5, etc., and so forth. Reference to a range of 2-125 therefore includes 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, and 125, as well as sub-ranges within the greater range, e.g., for 2-125, sub-ranges include but are not limited to 2-50, 5-50, 10-60, 5-45, 15-60, 10-40, 15-30, 2-85, 5-85, 20-75, 5-70, 10-70, 28-70, 14-56, 2-100, 5-100, 10-100, 5-90, 15-100, 10-75, 5-40, 2-105, 5-105, 100-95, 4-78, 15-65, 18-88, and 12-56. Reference to a range of 1-50 therefore includes 1, 2, 3, 4, 5, 6, 7, 8, 9, 10, 11, 12, 13, 14, 15, 16, 17, 18, 19, 20, etc., up to and including 50, as well as 1.1, 1.2, 1.3, 1.4, 1.5, etc., 2.1, 2.2, 2.3, 2.4, 2.5, etc., and so forth. Reference to a series of ranges includes ranges which combine the values of the boundaries of different ranges within the series. Thus, to illustrate reference to a series of ranges, for example, a range of 1-1,000 includes, for example, 1-10, 10-20, 20-30, 30-40, 40-50, 50-60, 60-75, 75-100, 100-150, 150-200, 200-250, 250-300, 300-400, 400-500, 500-750, 750-1,000, and includes ranges of 1-20, 10-50, 50-100, 100-500, and 500-1,000. The range 100 units to 2000 units therefore refers to and includes all values or ranges of values of the units, and fractions of the values of the units and integers within said range, including for example, but not limited to 100 units to 1000 units, 100 units to 500 units, 200 units to 1000 units, 300 units to 1500 units, 400 units to 2000 units, 500 units to 2000 units, 500 units to 1000 units, 250 units to 1750 units, 250 units to 1200 units, 750 units to 2000 units, 150 units to 1500 units, 100 units to 1250 units, and 800 units to 1200 units. Any two values within the range of about 100 units to about 2000 units therefore can be used to set the lower and upper boundaries of a range in accordance with the embodiments of the present disclosure. More particularly, a range of 10-12 units includes, for example, 10, 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9, and 12.0, and all values or ranges of values of the units, and fractions of the values of the units and integers within said range, and ranges which combine the values of the boundaries of different ranges within the series, e.g., 10.1 to 11.5. Reference to an integer with more (greater) or less than includes any number greater or less than the reference number, respectively. Thus, for example, reference to less than 100 includes 99, 98, 97, etc. all the way down to the number one (1); and less than 10 includes 9, 8, 7, etc. all the way down to the number one (1).

Polygenic risk scores (PRS) estimate the genetic risk of an individual for a complex disease based on many genetic variants across the whole genome. Provided herein is a deep neural network (DNN) that was found to outperform alternative machine learning techniques and established statistical algorithms, including BLUP, BayesA and LDpred. In the test cohort with 50% prevalence, the Area Under the receiver operating characteristic Curve (AUC) were 67.4% for DNN, 64.2% for BLUP, 64.5% for BayesA, and 62.4% for LDpred. BLUP, BayesA, and LPpred all generated PRS that followed a normal distribution in the case population. However, the PRS generated by DNN in the case population followed a bi-modal distribution composed of two normal distributions with distinctly different means. This suggests that DNN was able to separate the case population into a high-genetic-risk case sub-population with an average PRS significantly higher than the control population and a normal-genetic-risk case sub-population with an average PRS similar to the control population. This allowed DNN to achieve 18.8% recall at 90% precision in the test cohort with 50% prevalence, which can be extrapolated to 65.4% recall at 20% precision in a general population with 12% prevalence. Interpretation of the DNN model identified salient variants that were assigned insignificant p-values by association studies, but were important for DNN prediction. These variants may be associated with the phenotype through non-linear relationships.

While the present method is discussed in the context of breast cancer, the methods used herein can be applied in a variety disease, and in particular genetically complex diseases, such as, for example, other types of cancer, diabetes, neurological disorders, and neuromuscular disorders.

Deep learning generally refers to methods that map data through multiple levels of abstraction, where higher levels represent more abstract entities. The goal of deep learning is to provide a fully automatic system for learning complex functions that map inputs to outputs, without using hand crafted features or rules. One implementation of deep learning comes in the form of feedforward neural networks, where levels of abstraction are modeled by multiple non-linear hidden layers.

On average, SNPs can occur at approximately 1 in every 300 bases and as such there can be about 10 million SNPs in the human genome. In some cases, the deep neural network is trained with a labeled dataset comprising at least about 1,000, at least about 2,000, at least about 3,000, at least about 4,000, at least about 5,000, at least about 10,000, at least about 15,000, at least about 18,000, at least about 20,000, at least about 21,000, at least about 22,000, at least about 23,000, at least about 24,000, at least about 25,000, at least about 26,000, at least about 28,000, at least about 30,000, at least about 35,000, at least about 40,000, or at least about 50,000 SNPs.

In some cases, the neural network may be trained such that a desired accuracy of PRS calling is achieved (e.g., at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99%). The accuracy of PRS calling may be calculated as the percentage of patients with a known disease state that are correctly identified or classified as having or not have the disease.

In some cases, the neural network may be trained such that a desired sensitivity of PRS calling is achieved (e.g., at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99%). The sensitivity of PRS calling may be calculated as the percentage of patient’s having a disease that are correctly identified or classified as having the disease.

In some cases, the neural network may be trained such that a desired specificity of PRS calling is achieved (e.g., at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99%). The specificity of PRS calling may be calculated as the percentage of healthy patients that are correctly identified or classified as not having a disease.

In some cases, the methods, systems, and devices of the present disclosure are applicable to diagnose, prognosticate, or monitor disease progression in a subject. For example, a subject can be a human patient, such as a cancer patient, a patient at risk for cancer, a patient suspected of having cancer, or a patient with a family or personal history of cancer. The sample from the subject can be used to analyze whether or not the subject carries SNPs that are implicated in certain diseases or conditions, e.g., cancer, Neurofibromatosis 1, McCune-Albright, incontinentia pigmenti, paroxysmal nocturnal hemoglobinuria, Proteus syndrome, or Duchenne Muscular Dystrophy. The sample from the subject can be used to determine whether or not the subject carries SNPs and can be used to diagnose, prognosticate, or monitor any cancer, e.g., any cancer disclosed herein.

In another aspect, the present disclosure provides a method comprising determining a polygenic risk score for a subject, and diagnosing, prognosticating, or monitoring the disease in the subject. In some cases, the method further comprises providing treatment recommendations or preventative monitoring recommendations for the disease, e.g., the cancer. In some cases, the cancer is selected from the group consisting of: adrenal cancer, anal cancer, basal cell carcinoma, bile duct cancer, bladder cancer, cancer of the blood, bone cancer, a brain tumor, breast cancer, bronchus cancer, cancer of the cardiovascular system, cervical cancer, colon cancer, colorectal cancer, cancer of the digestive system, cancer of the endocrine system, endometrial cancer, esophageal cancer, eye cancer, gallbladder cancer, a gastrointestinal tumor, hepatocellular carcinoma, kidney cancer, hematopoietic malignancy, laryngeal cancer, leukemia, liver cancer, lung cancer, lymphoma, melanoma, mesothelioma, cancer of the muscular system, Myelodysplastic Syndrome (MDS), myeloma, nasal cavity cancer, nasopharyngeal cancer, cancer of the nervous system, cancer of the lymphatic system, oral cancer, oropharyngeal cancer, osteosarcoma, ovarian cancer, pancreatic cancer, penile cancer, pituitary tumors, prostate cancer, rectal cancer, renal pelvis cancer, cancer of the reproductive system, cancer of the respiratory system, sarcoma, salivary gland cancer, skeletal system cancer, skin cancer, small intestine cancer, stomach cancer, testicular cancer, throat cancer, thymus cancer, thyroid cancer, a tumor, cancer of the urinary system, uterine cancer, vaginal cancer, vulvar cancer, and any combination thereof.

In some cases, the determination of a PRS can provide valuable information for guiding the therapeutic intervention, e.g., for the cancer of the subject. For instance, SNPs can directly affect drug tolerance in many cancer types; therefore, understanding the underlying genetic variants can be useful for providing precision medical treatment of a cancer patient. In some cases, the methods, systems, and devices of the present disclosure can be used for application to drug development or developing a companion diagnostic. In some cases, the methods, systems, and devices of the present disclosure can also be used for predicting response to a therapy. In some cases, the methods, systems, and devices of the present disclosure can also be used for monitoring disease progression. In some cases, the methods, systems, and devices of the present disclosure can also be used for detecting relapse of a condition, e.g., cancer. A presence or absence of a known somatic variant or appearance of new somatic variant can be correlated with different stages of disease progression, e.g., different stages of cancers. As cancer progresses from early stage to late stage, an increased number or amount of new mutations can be detected by the methods, systems, or devices of the present disclosure.

Methods, systems, and devices of the present disclosure can be used to analyze biological sample from a subject. The subject can be any human being. The biological sample for PRF determination can be obtained from a tissue of interest, e.g., a pathological tissue, e.g., a tumor tissue. Alternatively, the biological sample can be a liquid biological sample containing cell-free nucleic acids, such as blood, plasma, serum, saliva, urine, amniotic fluid, pleural effusion, tears, seminal fluid, peritoneal fluid, and cerebrospinal fluid. Cell-free nucleic acids can comprise cell-free DNA or cell-free RNA. The cell-free nucleic acids used by methods and systems of the present disclosure can be nucleic acid molecules outside of cells in a biological sample. Cell-free DNA can occur naturally in the form of short fragments.

A subject applicable by the methods of the present disclosure can be of any age and can be an adult, infant or child. In some cases, the subject is within any age range (e.g., between 0 and 20 years old, between 20 and 40 years old, or between 40 and 90 years old, or even older). In some cases, the subject as described herein can be a non-human animal, such as non-human primate, pig, dog, cow, sheep, mouse, rat, horse, donkey, or camel.

The use of the deep neural network can be performed with a total computation time (e.g., runtime) of no more than about 7 days, no more than about 6 days, no more than about 5 days, no more than about 4 days, no more than about 3 days, no more than about 48 hours, no more than about 36 hours, no more than about 24 hours, no more than about 22 hours, no more than about 20 hours, no more than about 18 hours, no more than about 16 hours, no more than about 14 hours, no more than about 12 hours, no more than about 10 hours, no more than about 9 hours, no more than about 8 hours, no more than about 7 hours, no more than about 6 hours, no more than about 5 hours, no more than about 4 hours, no more than about 3 hours, no more than about 2 hours, no more than about 60 minutes, no more than about 45 minutes, no more than about 30 minutes, no more than about 20 minutes, no more than about 15 minutes, no more than about 10 minutes, or no more than about 5 minutes.

In some cases, the methods and systems of the present disclosure may be performed using a single-core or multi-core machine, such as a dual-core, 3-core, 4-core, 5-core, 6-core, 7-core, 8-core, 9-core, 10-core, 12-core, 14-core, 16-core, 18-core, 20-core, 22-core, 24-core, 26-core, 28-core, 30-core, 32-core, 34-core, 36-core, 38-core, 40-core, 42-core, 44-core, 46-core, 48-core, 50-core, 52-core, 54-core, 56-core, 58-core, 60-core, 62-core, 64-core, 96-core, 128-core, 256-core, 512-core, or 1,024-core machine, or a multi-core machine having more than 1,024 cores. In some cases, the methods and systems of the present disclosure may be performed using a distributed network, such as a cloud computing network, which is configured to provide a similar functionality as a single-core or multi-core machine.

Various aspects of the technology can be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that can bear the software elements includes optical, electrical and electromagnetic waves, such as used across physical interfaces between local devices, through wired and optical landline networks and over various air-links. The physical elements that carry such waves, such as wired or wireless links, optical links or the like, also can be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as can be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media can be involved in carrying one or more sequences of one or more instructions to a processor for execution.

Any of the methods described herein can be totally or partially performed with a computer system including one or more processors, which can be configured to perform the operations disclosed herein. Thus, embodiments can be directed to computer systems configured to perform the operations of any of the methods described herein, with different components performing a respective operation or a respective group of operations. Although presented as numbered operations, the operations of the methods disclosed herein can be performed at a same time or in a different order. Additionally, portions of these operations can be used with portions of other operations from other methods. Also, all or portions of an operation can be optional. Additionally, any of the operations of any of the methods can be performed with modules, units, circuits, or other approaches for performing these operations.

The present disclosure will now be discussed in terms of several specific, non-limiting, examples and embodiments. The examples described below, which include particular embodiments, will serve to illustrate the practice of the present disclosure, it being understood that the particulars shown are by way of example and for purposes of illustrative discussion of particular embodiments and are presented in the cause of providing what is believed to be a useful and readily understood description of procedures as well as of the principles and conceptual aspects of the present disclosure.

### Example 1 - Materials & Methods

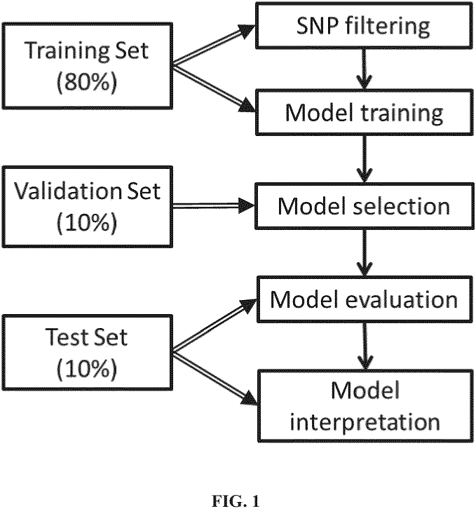

Breast cancer GWAS data. This study used a breast cancer genome-wide association study (GWAS) dataset generated by the Discovery, Biology, and Risk of Inherited Variants in Breast Cancer (DRIVE) project (Amos et al., 2017) and was obtained from the NIH dbGaP database under the accession number of phs001265.v1.pl. The DRIVE dataset was stored, processed and used on the Schooner supercomputer at the University of Oklahoma in an isolated partition with restricted access. The partition consisted of 5 computational nodes, each with 40 CPU cores (Intel Xeon Cascade Lake) and 200 GB of RAM. The DRIVE dataset in the dbGap database was composed of 49,111 subjects genotyped for 528,620 SNPs using OncoArray (Amos et al., 2017). 55.4% of the subjects were from North America, 43.3% from Europe, and 1.3% from Africa. The disease outcome of the subjects was labeled as malignant tumor (48%), in situ tumor (5%), and no tumor (47%). In this study, the subjects in the malignant tumor and in situ tumor categories were labeled as cases and the subjects in the no tumor category were labeled as controls, resulting in 26,053 (53%) cases and 23,058 (47%) controls. The subjects in the case and control classes were randomly assigned to a training set (80%), a validation set (10%), and a test set (10%) (FIG. 1). The association analysis was conducted on the training set using Plink 2.0 (Chang et al., 2015). For a subject, each of the 528,620 SNPs may take the value of 0, 1, or 2, representing the genotype value on a SNP for this subject. The value of 0 meant homozygous with minor allele, 1 meant heterozygous allele, and 2 meant homozygous with the dominant allele. Such encoding of the SNP information was also used in the following machine learning and statistical approaches. The p-value for each SNP was calculated using logistic regression in Plink 2.0.

Development of deep neural network models for PRS estimation. A variety of deep neural network (DNN) architectures (Bengio, 2009) were trained using Tensorflow 1.13. The Leaky Rectified Linear Unit (ReLU) activation function (Xu et al., 2019) was used on all hidden-layers neurons with the negative slope co-efficient set to 0.2. The output neuron used a sigmoid activation function. The training error was computed using the cross-entropy function:

$$\sum {}_{i = 1}^{n} y * \text{log} \left(p\right) + \left(1 - y\right) * \text{log} \left(1 - p\right) ,$$

where p ∈ [0,1] is the prediction probability from the model and y ∈ is the prediction target at 1 for case and 0 for control. The prediction probability was considered as the PRS from D NN.

DNNs were evaluated using different SNP feature sets. SNPs were filtered using their Plink association p-values at the thresholds of 10⁻², 10⁻³, 10⁻⁴ and 10⁻⁵. DNN was also tested using the full SNP feature set without any filtering. The DNN models were trained using mini-batches with a batch size of 512. The Adam optimizer (Kingma & Ba, 2019), an adaptive learning rate optimization algorithm, was used to update the weights in each mini-batch. The initial learning rate was set to 10⁻⁴,and the models were trained for up to 200 epochs with early stopping based on the validation AUC score. Dropout (Srivastava et al., 2014) was used to reduce overfitting. The dropout rates of 20%, 30%, 40%, 50%, 60%, 70%, 80%, and 90% were tested for the first hidden layer and the final dropout rate was selected based on the validation AUC score. The dropout rate was set to 50% on the other hidden layers in all architectures. Batch normalization (BN) (Ioffe & Szegedy, 2019) was used to accelerate the training process, and the momentum for the moving average was set to 0.9 in BN.

Development of alternative machine learning models for PRS estimation. Logistic regression, decision tree, random forest, AdaBoost, gradient boosting, support vector machine (SVM), and Gaussian naive Bayes were implemented and tested using the scikit-learn machine learning library in Python. These models were trained using the same training set as the DNNs and their hyperparameters were tuned using the same validation set based on the validation AUC (FIG. 1). These models are briefly described below.
Decision Tree: The gini information gain with best split was used. The maximum depth was not set to let the tree expanded until all leaves were pure or contained less than a minimum number of two examples per split (sklearn default parameters).
Random Forest: classification decision trees (as configured above) were used as base learners. The optimum number of decision trees were found to be 3,000 based on a parameter sweep between 500 and 5,000 with a step size of 500. Bootstrap samples were used to build each base learner. When searching for each tree’s best split, the maximum number of considered features was set to be the square root of the number of features.
AdaBoost: classification decision trees (as configured above) were used as base learners. The optimum number of decision trees were found to be 2,000 based on a parameter sweep between 500 and 5,000 with a step size of 500. The learning rate was set to 1. The algorithm used was SAMME.R (Hastie et al., 2009).
Gradient Boosting: regression decision trees (as configured above) were used as the base learners. The optimum number of decision trees were found to be 400 based on a parameter sweep between 100 and 1,000 with a step size of 100. Log-loss was used as the loss function. The learning rate was set to 0.1. The mean squared error with improvement score (Friedman, 2001) was used to measure the quality of a split.
SVM: The kernel was a radial basis function with
$$γ = \frac{1}{n * V a r}$$
, where n is the number of SNPs and Var is the variance of the SNPs across individuals. The regularization parameter C was set to 1 based on a parameter sweep over 0.001, 0.01, 0.1, 1, 5, 10, 15 and 20.
Logistic Regression: L2 regularization with a = 0.5 was used based on a parameter sweep for a over 0.0001, 0.001, 0.01, 0.1, 0.2, 0.3, 0.4, 0.5, 0.6, 0.7 and 0.8. L1 regularization was tested, but not used, because it did not improve the performance.
Gaussian Naive Bayes: The likelihood of the features was assumed to be Gaussian. The classes had uninformative priors.

Development of statistical models for PRS estimation. The same training and validation sets were used to develop statistical models (FIG. 1). The BLUP and BayesA models were constructed using the bWGR R package. The LDpred model was constructed as described (Vilhjalmsson et al., 2015).
BLUP: The linear mixed model was y = µ+ Xb + e , where y were the response variables, µ were the intercepts, X were the input features, b were the regression coefficients, and e were the residual coefficients.
BayesA: The priors were assigned from a mixture of normal distributions.
LDpred: The p-values were generated by our association analysis described above. The validation set was provided as reference for LDpred data coordination. The radius of the Gibbs sampler was set to be the number of SNPs divided by 3000 as recommended by the LDpred user manual (available at github.com/bvilhjal/ldpred/blob/master/ldpred/run.py).

The score distributions of DNN, BayesA, BLUP and LDpred were analyzed with the Shapiro test for normality and the Bayesian Gaussian Mixture (BGM) expectation maximization algorithm. The BGM algorithm decomposed a mixture of two Gaussian distributions with weight priors at 50% over a maximum of 1000 iterations and 100 initializations.

DNN model interpretation. LIME and DeepLift were used to interpret the DNN predictions for subjects in the test set with DNN output scores higher than 0.67, which corresponded to a precision of 90%. For LIME, the submodular pick algorithm was used, the kernel size was set to 40, and the number of explainable features was set to 41. For DeepLift, the importance of each SNPs was computed as the average across all individuals, and the reference activation value for a neuron was determined by the average value of all activations triggered across all subjects.

### Example 2 - Development of a Machine Learning Model for Breast Cancer PRS Estimation

The breast cancer GWAS dataset containing 26,053 cases and 23,058 controls was generated by the Discovery, Biology, and Risk of Inherited Variants in Breast Cancer (DRIVE) project (Amos et al., 2017). The DRIVE data is available from the NIH dbGaP database under the accession number of phs001265.v1.p1. The cases and controls were randomly split to a training set, a validation set, and a test set (FIG. 1). The training set was used to estimate p-values of SNPs using association analysis and train machine learning and statistical models. The hyperparameters of the machine learning and statistical models were optimized using the validation set. The test set was used for the final performance evaluation and model interpretation.

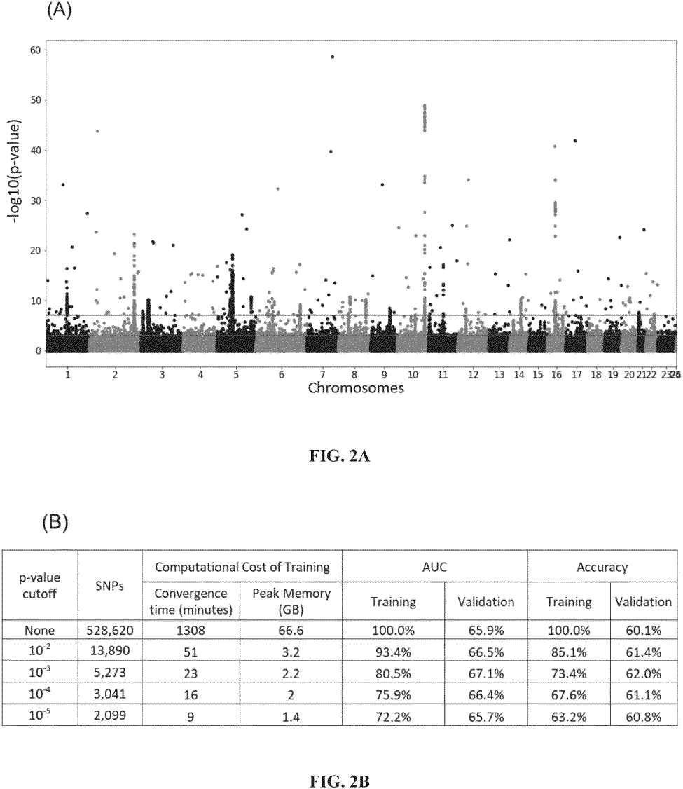

Statistical significance of the disease association with the 528,620 SNPs was assessed with Plink using only the training set. To obtain unbiased benchmarking results on the test set, it was critical not to use the test set in the association analysis (FIG. 1) and not to use association p-values from previous GWAS studies that included subjects in the test set, as well-described in the Section 7.10.2 of Hastie et al. (2009). The obtained p-values for all SNPs are shown in FIG. 2A as a Manhattan plot. There were 1,061 SNPs with a p-value less than the critical value of 9.5 - 10⁻⁸, which was set using the Bonferroni correction (9.5 ̇̇ 10⁻⁸ = 0.05/528,620). Filtering with a Bonferroni-corrected critical value may remove many informative SNPs that have small effects on the phenotype, epistatic interactions with other SNPs, or non-linear association with the phenotype (De et al., 2014). Relaxed filtering with higher p-value cutoffs was tested to find the optimal feature set for DNN (FIG. 2B and Tables 3A-E). The DNN models in FIG. 2B had a deep feedforward architecture consisting of an input layer of variable sizes, followed by 3 successive hidden layers containing 1000, 250, and 50 neurons, and finally an output layer with a single neuron. As the p-value cutoff increased, a greater number of SNPs were incorporated as input features, and training consumed a larger amount of computational resources in terms of computing time and peak memory usage. A feature set containing 5,273 SNPs above the p-value cutoff of 10⁻³ provided the best prediction performance measured by the AUC and accuracy on the validation set. In comparison with smaller feature sets from more stringent p-value filtering, the 5,273-SNP feature set may have included many informative SNPs providing additional signals to be captured by DNN for prediction. On the other hand, more relaxed filtering with p-value cutoffs greater than 10⁻³ led to significant overfitting as indicated by an increasing prediction performance in the training set and a decreasing performance in the validation set (FIG. 2B).

Previous studies (Khera et al., 2018; Gola et al., 2020) have used a large number of SNPs for PRS estimation on different datasets. In our study, the largest DNN model, consisting of all 528,620 SNPs, decreased the validation AUC score by 1.2% and the validation accuracy by 1.9% from the highest achieved values. This large DNN model relied an 80% dropout rate to obtain strong regularization, while all the other DNN models utilized a 50% dropout rate. This suggested that DNN was able to perform feature selection without using association p-values, although the limited training data and the large neural network size resulted in complete overfitting with a 100% training accuracy and the lowest validation accuracy (FIG. 2B).

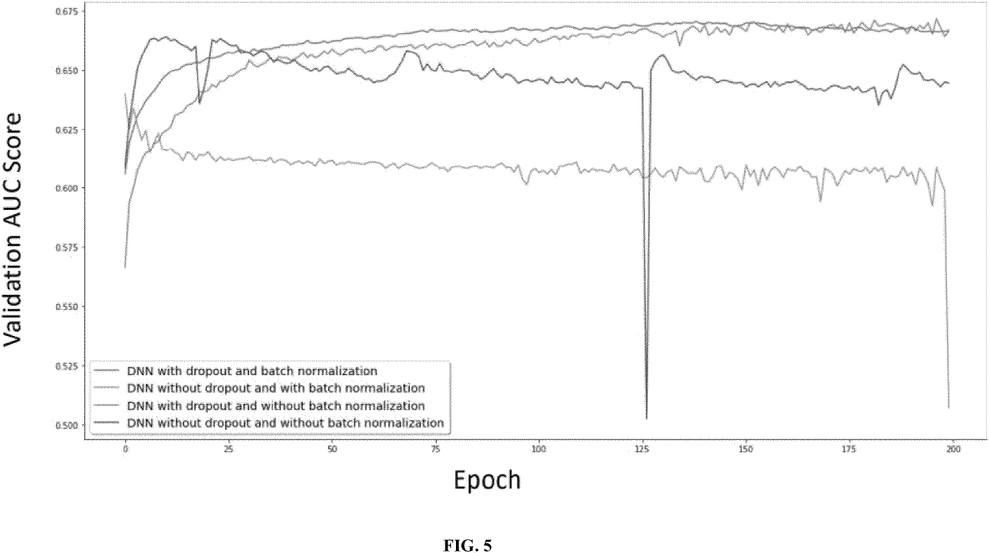

The effects of dropout and batch normalization were tested using the 5,273-SNP DNN model (FIG. 5). Without dropout, the DNN model using only batch normalization had a 3.0% drop in AUC and a 4.0% drop in accuracy and its training converged in only two epochs. Without batch normalization, the DNN model had 0.1% higher AUC and 0.3% lower accuracy but its training required a 73% increase in the number of epochs to reach convergence.

As an alternative to filtering, autoencoding was tested to reduce SNPs to a smaller set of encodings as described previously (Fergus et al., 2018; Cudie et al., 2018). An autoencoder was trained to encode 5273 SNPs into 2000 features with a mean square error (MSE) of 0.053 and a root mean square error (RMSE) of 0.23. The encodings from the autoencoder were used as the input features to train a DNN model with the same architecture as the ones shown in FIG. 2B except for the number of input neurons. The autoencoder-DNN model had a similar number of input neurons for DNN as the 2099-SNP DNN model, but had a 1.3% higher validation AUC and a 0.2% higher validation accuracy than the 2099-SNP DNN model (FIG. 2B). This increased validation AUC and accuracy suggested that the dimensionality reduction by the autoencoding from 5273 SNPs to 2000 encodings was better than the SNP filtering by the association p-values from 5273 SNPs to 2099 SNPs. However, the DNN models with 5,273 SNPs still had a 0.3% higher validation AUC score and a 1.6% higher validation accuracy than the autoencoder-DNN model.

The deep feedforward architecture benchmarked in FIG. 2B was compared with a number of alternative neural network architectures using the 5,273-SNP feature set (Table 4). A shallow neural network with only one hidden layer resulted in a 0.9% lower AUC and 1.1% lower accuracy in the validation set compared to the DNN. This suggested that additional hidden layers in DNN may allow additional feature selection and transformation in the model. One-dimensional convolutional neural network (ID CNN) was previously used to estimate the PRS for bone heel mineral density, body mass index, systolic blood pressure and waist-hip ratio (Bellot et al., 2018) and was also tested here for breast cancer prediction with the DRIVE dataset. The validation AUC and accuracy of ID CNN was lower than DNN by 3.2% and 2.0%, respectively. CNN was commonly used for image analysis, because the receptive field of the convolutional layer can capture space-invariant information with shared parameters. However, the SNPs distributed across a genome may not have significant space-invariant patterns to be captured by the convolutional layer, which may explain the poor performance of CNN.

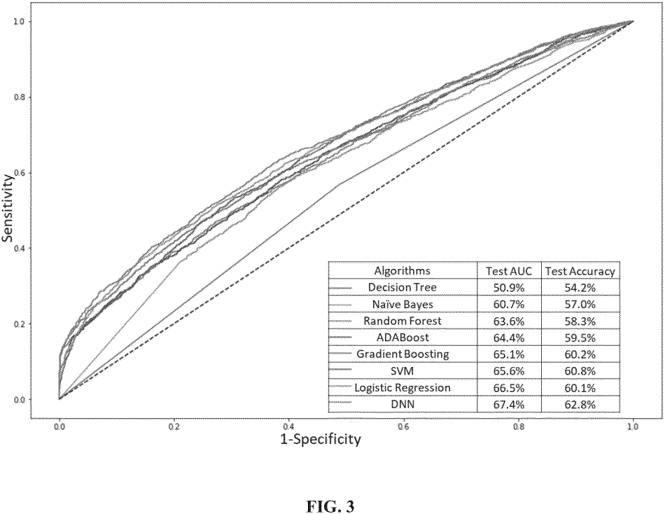

The 5,273-SNP feature set was used to test alternative machine learning approaches, including logistic regression, decision tree, naive Bayes, random forest, ADAboost, gradient boosting, and SVM, for PRS estimation (FIG. 3). These models were trained, turned, and benchmarked using the same training, validation, and test sets, respectively, as the DNN models (FIG. 1). Although the decision tree had a test AUC of only 50.9%, ensemble algorithms that used decision trees as the base learner, including random forest, ADABoost, and gradient boosting, reached test AUCs of 63.6%, 64.4%, and 65.1%, respectively. This showed the advantage of ensemble learning. SVM reached a test AUC of 65.6%. Naive Bayes and logistic regression were both linear models with the assumption of independent features. Logistic regression had higher AUC, but lower accuracy, than SVM and gradient boosting. The test AUC and test accuracy of DNN were higher than those of logistic regression by 0.9% and 2.7%, respectively. Out of all the machine learning models, the DNN model achieved the highest test AUC at 67.4% and the highest test accuracy at 62.8% (FIG. 3).

### Example 3 - Comparison of the DNN Model With Statistical Models for Breast Cancer PRS Estimation

The performance of DNN was compared with three representative statistical models, including BLUP, BayesA, and LDpred (Table 1). Because the relative performance of these methods may be dependent on the number of training examples available, the original training set containing 39,289 subjects was down-sampled to create three smaller training sets containing 10,000, 20,000, and 30,000 subjects. As the 5,273-SNP feature set generated with a p-value cutoff of 10⁻³ may not be the most appropriate for the statistical methods, a 13,890-SNP feature set (p-value cutoff = 10-² ) and a 2,099-SNP feature set (p-value cutoff = 10⁻⁵) were tested for all methods.

Although LDpred also required training data, its prediction relied primarily on the provided p-values, which were generated for all methods using all 39,289 subjects in the training set. Thus, the down-sampling of the training set did not reduce the performance of LDpred. LDpred reached its highest AUC score at 62.4% using the p-value cutoff of 10⁻³. A previous study (Ge et al., 2019) that applied LDpred to breast cancer prediction using the UK Biobank dataset similarly obtained an AUC score of 62.4% at the p-value cutoff of 10⁻³ This showed consistent performance of LDpred in the two studies. When DNN, BLUP, and BayesA used the full training set, they obtained higher AUCs than LDpred at their optimum p-value cutoffs.

DNN, BLUP, and BayesA all gained performance with the increase in the training set sizes (Table 1). The performance gain was more substantial for DNN than BLUP and BayesA. The increase from 10,000 subjects to 39,258 subjects in the training set resulted in a 1.9% boost to DNN’s best AUC, a 0.7% boost to BLUP, and a 0.8% boost to BayesA. This indicated the different variance-bias trade-offs made by DNN, BLUP, and BayesA. The high variance of DNN required more training data, but could capture non-linear relationships between the SNPs and the phenotype. The high bias of BLUP and BayesA had lower risk for overfitting using smaller training sets, but their models only considered linear relationships. The higher AUCs of DNN across all training set sizes indicated that DNN had a better variance-bias balance for breast cancer PRS estimation.

For all four training set sizes, BLUP and BayesA achieved higher AUCs using more stringent p-value filtering. When using the full training set, reducing the p-value cutoffs from 10⁻² to 10⁻⁵ increased the AUCs of BLUP from 61.0% to 64.2% and the AUCs of BayesA from 61.1% to 64.5%. This suggested that BLUP and BayesA preferred a reduced number of SNPs that were significantly associated with the phenotype. On the other hand, DNN produced lower AUCs using the p-value cutoff of 10⁻⁵ than the other two higher cutoffs. This suggested that DNN can perform better feature selection in comparison to SNP filtering based on association p-values.

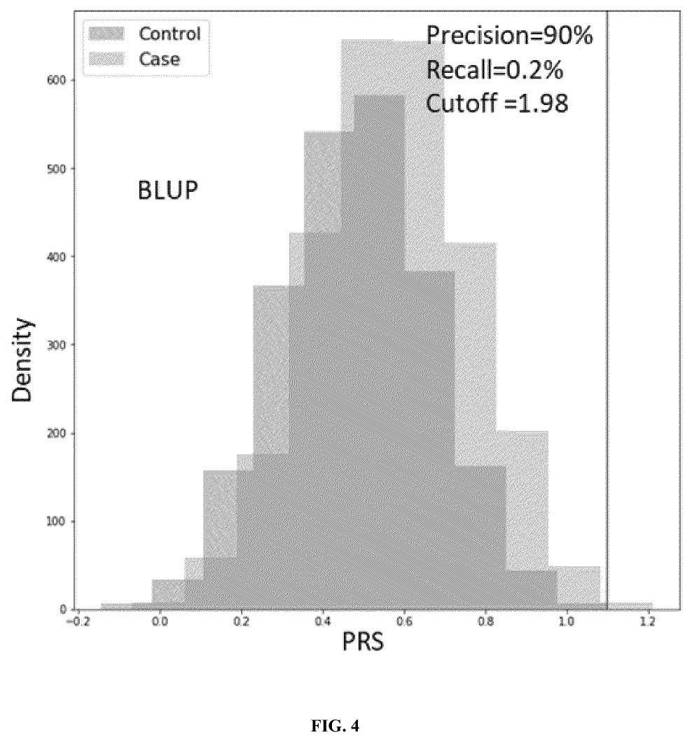

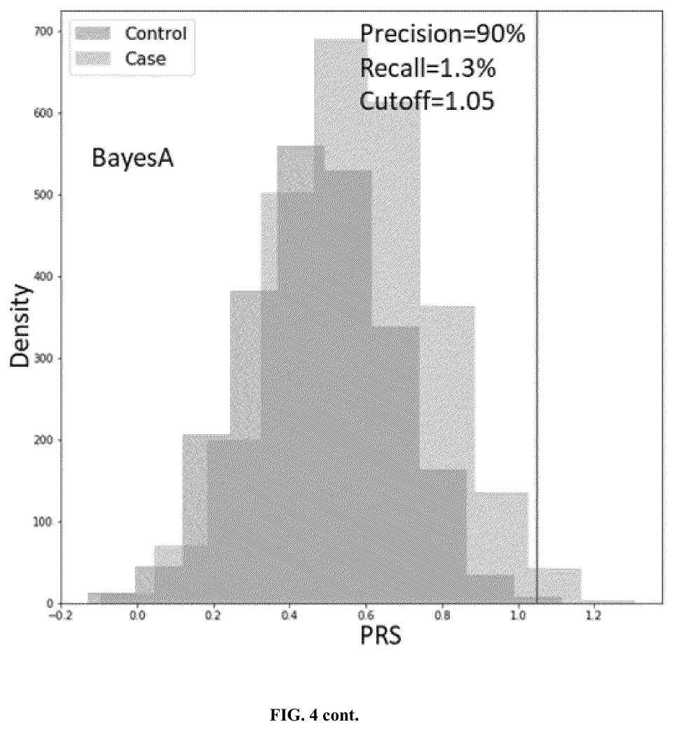

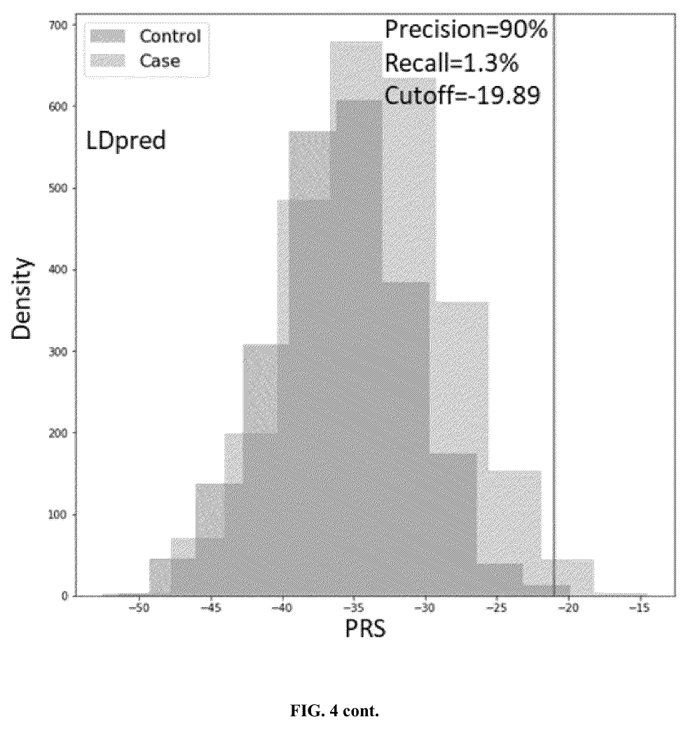

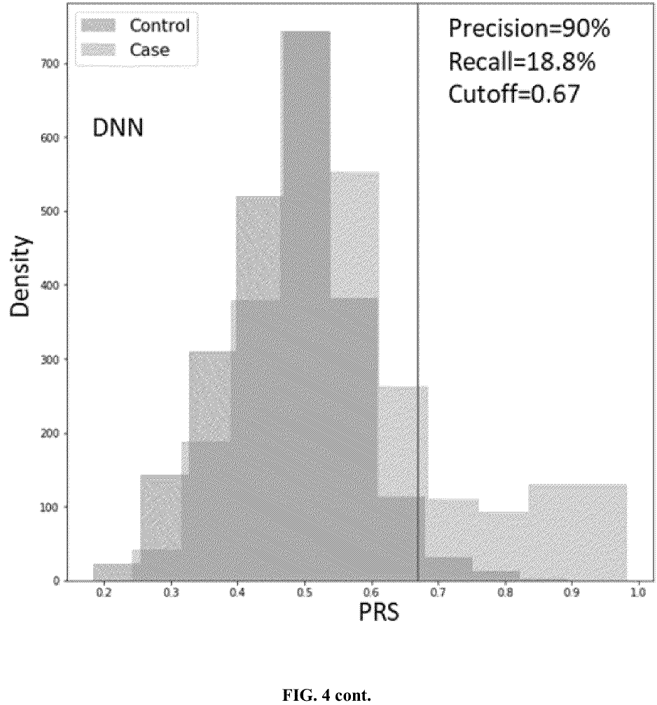

The four algorithms were compared using the PRS histograms of the case population and the control population from the test set in FIG. 4. The score distributions of BLUP, BayesA, and LDpred all followed normal distributions. The p-values from the Shapiro normality test of the case and control distributions were 0.46 and 0.43 for BayesA, 0.50 and 0.95 for BLUP, and 0.17 and 0.24 for LDpred, respectively. The case and control distributions were ^{N}_{case}^{(µ}= 0.577, σ= 0.20) and N_{control}(µ= 0.479, σ= 0.19) from BayesA, N_{case}(µ = 0.572, σ= 0.19) and ^{N}_{control}^{(µ}= 0.483, σ= 0.18) from BLUP, and ^{N}_{case}^{(µ} = -33.52, σ= 5.4) and ^{N}_{control}^{(µ} = -35.86, σ= 4.75) from LDpred. The means of the case distributions were all significantly higher than the control distributions for BayesA (p-value < 10⁻¹⁶), BLUP (p-value < 10⁻¹⁶), and LDpred (p-value < 10⁻¹⁶), and their case and control distributions had similar standard deviations.

The score histograms of DNN did not follow normal distributions based on the Shapiro normality test with a p-value of 4.1 _{*} 10⁻³⁴ for the case distribution and a p-value of 2.5 _{*} 10⁻⁹ for the control distribution. The case distribution had the appearance of a bi-modal distribution. The Bayesian Gaussian mixture expectation maximization algorithm decomposed the case distribution to two normal distributions: ^{N}_{case1}^{(µ} = 0.519, σ= 0.096) with an 86.5% weight and N_{case2}^{(µ}= 0.876, σ= 0.065) with a 13.5% weight. The control distribution was resolved into two normal distributions with similar means and distinct standard deviations: ^{N}_{control1}^{(µ}= 0.471, σ = 0.1) with an 85.0% weight and N_{control2}(µ= 0.507, σ = 0.03) with a 15.0% weight. The N_{case1} distribution had a similar mean as the ^{N}_{control1} and N_{control2} distributions. This suggested that the N_{case1} distribution may represent a normal-genetic-risk case sub-population, in which the subjects may have a normal level of genetic risk for breast cancer and the oncogenesis likely involved a significant environmental component. The mean of the N_{case2} distribution was higher than the means of both the N_{case1} and N_{control1} distributions by more than 4 standard deviations (p-value < 10⁻¹⁶). Thus, the N_{case2} distribution likely represented a high-genetic-risk case sub-population for breast cancer, in which the subjects may have inherited many genetic variations associated with breast cancer.

Three GWAS were performed between the high-genetic-risk case sub-population with DNN PRS > 0.67, the normal-genetic-risk case sub-population with DNN PRS < 0.67, and the control population (Table 5). The GWAS analysis of the high-genetic-risk case sub-population versus the control population identified 182 significant SNPs at the Bonferroni level of statistical significance. The GWAS analysis of the high-genetic-risk case sub-population versus the normal-genetic-risk case sub-population identified 216 significant SNPs. The two sets of significant SNPs found by these two GWAS analyses were very similar, sharing 149 significant SNPs in their intersection. Genes associated with these 149 SNPs were investigated with pathway enrichment analysis (Fisher’s Exact Test; P < 0.05) using SNPnexus (Dayem et al., 2018) (Table 6). Many of the significant pathways were involved in DNA repair (O’Connor, 2015), signal transduction (Kolch et al., 2015), and suppression of apoptosis (Fernald & Kurokawa, 2013). Interestingly, the GWAS analysis of the normal-genetic-risk case sub-population and the control population identified no significant SNP. This supported the classification of the cases into the normal-genetic-risk subjects and the high-genetic-risk subjects based on their PRS scores from the DNN model.

In comparison with AUCs, it may be more relevant for practical applications of PRS to compare the recalls of different algorithms at a given precision that warrants clinical recommendations. At 90% precision, the recalls were 18.8% for DNN, 0.2% for BLUP, 1.3% for BayesA, and 1.3% for LDpred in the test set of the DRIVE cohort with a ∼50% prevalence. This indicated that DNN can make a positive prediction for 18.8% of the subjects in the DRIVE cohort and these positive subjects would have an average chance of 90% to eventually develop breast cancer. American Cancer Society advises yearly breast MRI and mammogram starting at the age of 30 years for women with a lifetime risk of breast cancer greater than 20%, which meant a 20% precision for PRS. By extrapolating the performance in the DRIVE cohort, the DNN model should be able to achieve a recall of 65.4% at a precision of 20% in the general population with a 12% prevalence rate of breast cancer.

TABLE 1

AUC test scores of DNN, BLUP, BayesA and LDpred models at different p-value cutoffs (PC) and training set sizes (TS)

| | DNN | | | BLUP | | | BayesA | | | LDpred | | |
|---|---|---|---|---|---|---|---|---|---|---|---|---|
| | 10⁻⁵* | 10⁻³* | 10⁻²* | 10⁻⁵* | 10⁻³* | 10⁻²* | 10⁻⁵* | 10⁻³* | 10⁻²* | 10⁻⁵* | 10⁻³* | 10⁻²* |
| 10,000** | 64.8% | 65.5% | 65.1% | 63.5% | 62.5% | 60.6% | 63.7% | 62.0% | 59.9% | 60.8% | 62.4% | 61.6% |
| 20,000*** | 65.6% | 66.6% | 66.4% | 62.9% | 63.0% | 60.6% | 62.7% | 63.0% | 60.4% | 60.8% | 62.4% | 61.6% |
| 30,000** | 66.0% | 66.9% | 66.6% | 64.2% | 63.1% | 60.7% | 64.3% | 63.1% | 60.7% | 60.7% | 62.4% | 61.6% |
| 39,289** | 66.2% | 67.4% | 67.3% | 64.2% | 63.3% | 61.0% | 64.5% | 63.4% | 61.1% | 60.7% | 62.4% | 61.6% |

*: p-value cutoff
**: training set size

TABLE 2

Top salient SNPs identified by both LIME and DeepLift from the DNN model

| SNP | locus | LIME (10⁻⁴) | DeepLift (10⁻²) | p-value | MAF* | Genes of interest** |
|---|---|---|---|---|---|---|
| corect_rs139337779 | 12q24.22 | 4.5 | -3.3 | 6.5E-04 | 11% | NOS1 |
| chr13_113796587A G | 3q34 | 4.3 | -3.8 | 2.8E-04 | 3% | F10 |
| chr9_16917672_G_T | 9p22.2 | 4.5 | -2.5 | 7.6E-05 | 4% | BNC2/RP11-132E11.2 |
| chr8_89514784_A_G | 8q21.3 | 27.0 | 3.7 | 2.5E--05 | 56% | RP11-586K2.1 |
| chr17_4961271_G_T | 17p13.2 | 4.2 | -2.2 | 8.2E-06 | 4% | SLC52A1/RP11-46l8.1 |
| rs11642757 | 16q23.2 | 5.3 | -2.9 | 2.0E-06 | 6％ | RP11-345M22.1 |
| rs404605 | 1p36.33 | 4.4 | 2.4 | 9.6E-07 | 37% | RP11-5407.3/SAMD11 |
| chr5_180405432_G_Thr5 180405432_G_T | 5q35.3 | 4.1 | -3.4 | 2.3E-07 | 3% | CTD-2593A12.3/CTD-2593A12.4 |
| Chr6:30954121:G:T | 6p21.33 | 6.8 | 4.9 | 1.0E-08 | 42% | MUC21 |
| chrl4_101121371_G_T | 14q32.2 | 5.8 | 3.9 | 1.0E-10 | 33% | CTD-2644121.1/LINC00523 |
| rsl2542492 | 8q21.11 | 40.0 | 2.8 | 6.3E-11 | 34% | RP11-434l12.2 |
| corect_rs116995945 | 17q22 | 3.6 | -4.5 | 2.5E-11 | 5% | SCPEP1/RNF126P1 |
| chr14_76886176C_T | 14q24.3 | 3.5 | 2.3 | 2.3E-11 | 41% | ESRRB |
| chr2_171708059_C_T | 2q31.1 | 4.1 | -6.7 | 1.9E-11 | 7% | GAD1 |
| chr7_102368966_A_G | 7q22.1 | 4.1 | -2.6 | 6.8E-12 | 4% | RA5A4DP/FAM 185A |
| chr8_130380476_C_T | 8q24.21 | 4.3 | 2.5 | 4.7E-12 | 22% | CCDC26 |
| corect_rs181578054 | 22q13.33 | 4.1 | 3.0 | 7.1E-14 | 40% | ARSA/YRNA |
| rs3858522 | 11p15.5 | 7.7 | 3.3 | 2.2E-17 | 52% | H19/IGF2 |
| chr3_46742523_A_C | 3p21.31 | 5.2 | 4.9 | 1.8E-22 | 35% | ALS2CL/TMIE |
| r13_113284191_C_T13_113284191_C_T | 13q34 | 4.0 | -4.0 | 7.8E-23 | 5% | TUBGCP3/Cl3orf35 |
| chr1_97788840_A_G | 1p21.3 | 6.0 | -6.8 | 6.6E-34 | 9% | DPYD |
| chr7_118831547_C_T | 7q31.31 | 4.0 | -3.5 | 1.9E-40 | 6% | RP11-500M10.1 /AC091320.2 |
| chr6_52328666_C_T | 16q12.1 | 23.0 | 5.2 | 1.5E-41 | 21% | RP11-142G1.2/TOX3 |

*Minor Allele Frequency
** < 300 kb from target SNPs

TABLE 3A

Performance benchmarking of a 528,620-SNP DNN Model (no SNP p-value cutoff)

| Threshold = 0.449 | | Actual labels | | Threshold = 0.682 | | Actual labels | |
|---|---|---|---|---|---|---|---|
| | | Case | Control | | | Case | Control |
| Predicted labels | Case | 2354 | 1814 | Predicted labels | Case | 362 | 41 |
| | Control | 263 | 481 | | Control | 2255 | 2254 |
| Performance Measure | | Value | | Performance Measure | | Value | |
| Sensitivity (recall) | | 90.0% | | Sensitivity (recall) | | 13.8% | |
| Specificity | | 21.0% | | Specificity | | 98.2% | |
| Precision | | 56.5% | | Precision | | 90.0% | |
| Negative predictive value | | 64.7% | | Negative predictive value | | 50.0% | |

TABLE 3B

Performance benchmarking of a 13,890-SNP DNN Model (SNP p-value cutoff = 10⁻²)

| Threshold = 0.385 | | Actual labels | | Threshold = 0.736 | | Actual labels | |
|---|---|---|---|---|---|---|---|
| | | Case | Control | | | Case | Control |
| Predicted labels | Case | 2352 | 1803 | Predicted labels | Case | 431 | 49 |
| | Control | 265 | 492 | | Control | 2186 | 2246 |

| Performance Measure | Value | Performance Measure | Value |
|---|---|---|---|
| Sensitivity (recall) | 16.5% | Sensitivity (recall) | 16.5% |
| Specificity | 97.9% | Specificity | 97.9% |
| Precision | 90.0% | Precision | 90.0% |
| Negative predictive value | 50.7% | Negative predictive value | 50.7% |

TABLE 3C

Performance benchmarking of a 5,273-SNP DNN Model (SNP p-value cutoff = 10⁻³)

| Threshold = 0.4 | | Actual labels | | Threshold = 0.68 | | Actual labels | |
|---|---|---|---|---|---|---|---|
| | | Case | Control | | | Case | Control |
| Predicted labels | Case | 2349 | 1784 | Predicted labels | Case | 444 | 51 |
| | Control | 268 | 511 | | Control | 2173 | 2244 |

| Performance Measure | Value | Performance Measure | Value |
|---|---|---|---|
| Sensitivity (recall) | 90.0% | Sensitivity (recall) | 17.0% |
| Specificity | 22.3% | Specificity | 97.8% |
| Precision | 56.8% | Precision | 90.0% |
| Negative predictive value | 65.6% | Negative predictive value | 50.8% |

TABLE 3D

Performance benchmarking of a 3,041-SNP DNN Model (SNP p-value cutoff = 10⁻⁴)

| Threshold = 0.401 | | Actual labels | | Threshold = 0.645 | | Actual labels | |
|---|---|---|---|---|---|---|---|
| | | Case | Control | | | Case | Control |
| Predicted labels | Case | 2350 | 1806 | Predicted labels | Case | 422 | 50 |
| | Control | 267 | 486 | | Control | 2195 | 2245 |

| Performance Measure | Value | Performance Measure | Value |
|---|---|---|---|
| Sensitivity (recall) | 90.0% | Sensitivity (recall) | 16.0% |
| Specificity | 21.3% | Specificity | 97.8% |
| Precision | 56.5% | Precision | 90.0% |
| Negative predictive value | 64.7% | Negative predictive value | 50.5% |

TABLE 3E

Performance benchmarking of a 2,099-SNP DNN Model (SNP p-value cutoff = 10⁻⁵)

| Threshold = 0.413 | | Actual labels | | Threshold = 0.644 | | Actual labels | |
|---|---|---|---|---|---|---|---|
| | | Case | Control | | | Case | Control |
| Predicted labels | Case | 2350 | 1792 | Predicted labels | Case | 391 | 46 |
| | Control | 267 | 503 | | Control | 2226 | 2249 |

| Performance Measure | Value | Performance Measure | Value |
|---|---|---|---|
| Sensitivity (recall) | 90.0% | Sensitivity (recall) | 14.8% |
| Specificity | 21.9% | Specificity | 98.0% |
| Precision | 56.7% | Precision | 90.0% |
| Negative predictive value | 65.3% | Negative predictive value | 50.3% |

TABLE 4

Comparison of neural network (NN) architectures

| Model | Architecture | Validation AUC | Validation Accuracy | Convergence (#Epoches) |
|---|---|---|---|---|
| DNN | 3 hidden layers with 1000, 250, and 50 neurons. Dropout and batch normalization (BN) enabled | 67.1% | 62.0% | 110 |
| Shallow NN (SNN) | 1 hidden layer with 50 neurons. With dropout but no BN | 66.2% | 60.9% | 20 |
| 1D Convolutional NN (1D CNN) | 2 convolution layers with max pooling followed by 3 hidden layers with 1000, 250, and 50 neurons. Dropout and BN enabled | 63.9% | 59.9% | 155 |
| Autoencoder-DNN | autoencoding with no hidden layer followed by DNN with dropout and BN enabled | 67.0% | 61.0% | 31 |

TABLE 5

GWAS between the high-genetic-risk case sub-population, the normal-genetic-risk case sub-population, and the control population
High-genetic-risk case sub-population vs. normal-genetic-risk case sub-population

| SNP | Chr. | Position | p.value | Genes* |
|---|---|---|---|---|
| rs609805 | 1 | 1226889 | 4.80E-08 | SCNN1D |
| chr1_1914124_C_T | 1 | 1914124 | 9.22E-11 | Clorf222 |
| rs74820022 | 1 | 3408706 | 5.25E-10 | MEGF6 |
| chr1_10617906_A_T | 1 | 10617906 | 1.93E-11 | PEX14 |
| chr1_15348453_A_C | 1 | 15348453 | 3.09E-14 | KAZN |
| rs602946 | 1 | 20915535 | 1.02E-09 | CDA |
| chr1_ 28632870_A_C | 1 | 28632870 | 4.38E-08 | SESN2,MED18 |
| rs4316319 | 1 | 78810600 | 2.28E-08 | PTGFR |
| chr1_97788840_A_G | 1 | 97788840 | 7.62E-18 | DPYD |
| chr1_114136037_C_T | 1 | 114136037 | 7.18E-09 | MAGI3 |
| rs1884296 | 1 | 115235716 | 1.49E-11 | AMPD1 |
| chr1_171056203_C_T | 1 | 171056203 | 2.62E-18 | RP5-1092L12.2,FMO3 |
| chr1_202172594_C_T | 1 | 202172594 | 7.70E-09 | LGR6 |
| chr1_204008939_C_T | 1 | 204008939 | 2.24E-09 | LINC00303 |
| rs729125 | 1 | 238118749 | 1.12E-14 | MTND5P18, YWHAQP9 |
| corect_rs189944458 | 2 | 18059890 | 1.67E-10 | KCNS3 |
| rs10193919 | 2 | 20880833 | 2.17E-08 | AC012065.7,C2orf43 |
| chr2_23168305_A_G | 2 | 23168305 | 2.49E-09 | RN7SKP27,AC016768.1 |
| chr2_23222481_C_T | 2 | 23222481 | 4.17E-15 | RN7SKP27,AC016768.1 |
| chr2_26526169_A_G | 2 | 26526169 | 1.41E-08 | HADHB,GPR113 |
| chr2_28150862_A_C | 2 | 28150862 | 3.84E-16 | BRE,MRPL33 |
| chr2_29009089_A_C | 2 | 29009089 | 2.84E-10 | PPP1CB,SPDYA |
| chr2_85901719_A_G | 2 | 85901719 | 7.33E-08 | SFTPB,GNLY |
| chr2_111862303_C_T | 2 | 111862303 | 1.21E-24 | AC096670.3,ACOXL |
| chr2_120189404_A_G | 2 | 120189404 | 6.71E-08 | TMEM37 |
| rs4988235 | 2 | 136608646 | 9.95E-08 | MCM6 |
| chr2_150721127_A_C | 2 | 150721127 | 1.36E-08 | AC007364.1,AC016682.1 |
| chr2_172017549_G_T | 2 | 172017549 | 1.24E-10 | TLK1 |
| exm-rs6707846 | 2 | 191286516 | 2.92E-08 | NA |
| chr2_192542793_C_G | 2 | 192542793 | 5.84E-08 | MYO1B,NABP1 |
| rs74948099 | 2 | 231010534 | 2.85E-09 | AC009950.2 |
| corect_rs187745955 | 3 | 20612509 | 9.52E-10 | RNU6-815P, AC104441.1 |
| rs9851291 | 3 | 20994957 | 2.98E-12 | RNU6-815P, AC104441.1 |
| chr3_28889125_C_T | 3 | 28889125 | 8.80E-10 | LlNC00693,AC097361.1 |
| rs2343912 | 3 | 32445089 | 3.30E-11 | CMTM7 |
| rs9813107 | 3 | 40987921 | 1.01E-13 | RP11-761N21.1,RP11-520A21.1 |
| chr3_46742523_A_C | 3 | 46742523 | 3.50E-22 | ALS2CL,TMIE |
| chr3_49501384_C_T | 3 | 49501384 | 2.25E-11 | NICN1,RNA5SP130 |
| chr3_50192826_C_T | 3 | 50192826 | 7.03E-15 | RP11-493K19.3,SEMA3F |
| chr3_53880367_G_T | 3 | 53880367 | 2.55E-11 | CHDH |
| rs13098429 | 3 | 114875160 | 2.25E-14 | ZBTB20,RP11-190P13.2 |
| chr3_138459216_A_G | 3 | 138459216 | 1.57E-11 | PIK3CB |
| rs11925421 | 3 | 145888162 | 9.82E-14 | PLOD2,PLSCR4 |
| chr3_149390610_A_T | 3 | 149390610 | 3.66E-14 | WWTR1 |
| chr3_149688990_A_G | 3 | 149688990 | 1.83E-10 | PFN2 |
| rs9866700 | 3 | 180281446 | 2.34E-08 | U8,RP11-496B10.3 |
| chr4_8182559_C_T | 4 | 8182559 | 6.67E-08 | GMPSP1,SH3TC1 |
| rs77204838 | 4 | 8605475 | 4.20E-17 | CPZ,GPR78 |
| chr4_9462484_G_T | 4 | 9462484 | 4.19E-18 | OR7E86P,OR7E84P |
| chr4_16013048_C_T | 4 | 16013048 | 1.12E-10 | PROM1 |
| chr4_39691575_G_T | 4 | 39691575 | 7.10E-08 | RP11-539G18.2,UBE2K |
| rs11735107 | 4 | 40038146 | 1.12E-10 | KRT18P25,RP11-333E13.4 |
| kgp21013528 | 4 | 46152421 | 4.63E-08 | NA |
| rs10518461 | 4 | 126164298 | 6.18E-11 | ANKRD50,FAT4 |
| rs73859240 | 4 | 162446738 | 3.01E-09 | FSTL5 |
| corect_rs112923443 | 4 | 172579034 | 3.07E-09 | RP11-97E7.2,GALNTL6 |
| rs3922497 | 4 | 190170679 | 1.28E-08 | RP11-706F1.1,RP11-706F1.2 |
| chr5_521096_C_T | 5 | 521096 | 2.89E-11 | SLC9A3 |
| chr5_524827_A_G | 5 | 524827 | 4.99E-08 | RP11-310P5.2 |
| chr5_770093_A_G | 5 | 770093 | 4.22E-12 | ZDHHC11 |
| rs456752 | 5 | 1484826 | 3.40E-12 | LPCAT1 |
| chr5_26168640_C_T | 5 | 26168640 | 3.08E-21 | RNU4-43P,RP 11 -3 51N6.1 |
| chr5_49502516_C_T | 5 | 49502516 | 3.37E-13 | CTD-2013M15.001,EMB |
| chr5_67103091_A_C | 5 | 67103091 | 1.96E-11 | RP11-434D9.2 |
| rs554514 | 5 | 111130274 | 7.17E-22 | NREP |
| chr5_116877991_A_C | 5 | 116877991 | 1.53E-15 | LINC00992 |
| rs801752 | 5 | 134819978 | 7.26E-16 | CTB-138E-5.1, NEUROG1 |
| rs1990941 | 5 | 164991054 | 2.23E-09 | CTC-535M15.2 |
| rs1736999 | 6 | 29764656 | 2.25E-12 | HLA-V |
| rs1633097 | 6 | 29784192 | 1.17E-08 | MICG,HLA-G |
| chr6_30243235_C_T | 6 | 30243235 | 1.01E-09 | HCG17,HLA-L |
| rs130065 | 6 | 31122500 | 7.49E-20 | CCHCR1 |
| chr6_31248091_A_G | 6 | 31248091 | 9.09E-08 | USP8P1,RPL3P2 |
| rs2523545 | 6 | 31333499 | 7.37E-10 | XXbac-BPG248L24.12,DHFRP2 |
| rs805288 | 6 | 31678028 | 1.83E-10 | MEGT1,LINC00908 |
| chr6_32470283_A_C | 6 | 32470283 | 7.86E-08 | HLA-DRB9,HLA-DRB5 |
| chr6_32480507_C_T | 6 | 32480507 | 2.82E-19 | HLA-DRB9,HLA-DRB5 |
| chr6_32484554_A_T | 6 | 32484554 | 1.30E-11 | HLA-DRB9,HLA-DRB5 |
| chr6_32494206_A_C | 6 | 32494206 | 5.51E-08 | HLA-DRB5 |
| chr6_32552168_A_G | 6 | 32552168 | 1.84E-14 | HLA-DRB1 |
| rs9271611 | 6 | 32591609 | 1.40E-10 | HLA-DRB1,HLA-DQA1 |
| chr6_32691173_C_T | 6 | 32691173 | 8.74E-11 | XXbac-BPG254F23.7,HLA-DQB3 |
| rs9275851 | 6 | 32691186 | 2.91E-18 | XXbac-BPG254F23.7,HLA-DQB3 |
| rs7753169 | 6 | 36614326 | 9.24E-21 | RNU1-88P,Y RNA |
| chr6_75480993_GT_I NDEL | 6 | 75480993 | 1.60E-09 | RP11-554D15.3,RP11-560O20.1 |
| chr6_93389344_C_T | 6 | 93389344 | 7.77E-08 | AL359987.1,RP11-127B16.1 |
| chr6_117598048_A_G | 6 | 117598048 | 4.32E-09 | VGLL2,ROS1 |
| rs79830246 | 6 | 151380101 | 8.83E-13 | MTHFD1L |
| chr6_153077792_A_G | 6 | 153077792 | 4.99E-12 | VIP |
| rs67465115 | 6 | 160267829 | 5.96E-08 | NA |
| chr6_161398697_G_I NDEL | 6 | 161398697 | 9.07E-10 | RP3-428L16.1,RP3-428L16.2 |
| rs61729932 | 7 | 2577816 | 4.05E-09 | BRAT1 |
| chr7_4832371_A_G | 7 | 4832371 | 2.67E-11 | AP5Z1 |
| corect_rs117345894 | 7 | 5470678 | 2.22E-10 | RP11-1275H24.3,FBXL18 |
| rs28379235 | 7 | 16129153 | 5.97E-19 | AC006035.1 |
| rs4724080 | 7 | 41949635 | 5.49E-08 | IN-HBA-AS1,GLI3 |
| chr7_91782274_A_G | 7 | 91782274 | 1.63E-11 | CTB-161K23.1,LRRD1 |
| rs58348977 | 7 | 99188014 | 3.17E-08 | GS1-259H13.10 |
| chr7_118831547_C_T | 7 | 118831547 | 2.52E-29 | RP11-500M10.1, AC091320.2 |
| rs10464695 | 7 | 128766463 | 7.67E-09 | CYCSP20,RP11-286H14.4 |
| rs62489409 | 7 | 129632800 | 2.34E-29 | RP11-306G20.1 |
| rs3927319 | 7 | 141248158 | 3.49E-09 | RP11-744I24.2 |
| rs10099442 | 8 | 1792759 | 7.47E-08 | ARHGEF10 |
| rs7821602 | 8 | 5220317 | 4.05E-11 | RN7SL318P,RP11-745K9.2 |
| chr8_17265628_A_G | 8 | 17265628 | 2.95E-12 | MTMR7 |
| chr8_21408145_C_T | 8 | 21408145 | 2.21E-09 | AC022716.1,GFRA2 |
| chr8_87816647_C_T | 8 | 87816647 | 2.95E-08 | RP11-386D6.2 |
| chr8_128146308_G_T | 8 | 128146308 | 2.69E-10 | PRNCR1,CASC19 |
| rs7856798 | 9 | 5952026 | 8.35E-08 | KIAA2026 |
| rs2578291 | 9 | 6642973 | 1.66E-08 | GLDC |
| rs1180130 | 9 | 72904219 | 9.35E-09 | SMC5 |
| rs59032320 | 9 | 109874246 | 3.53E-09 | RP11-508N12.2,RP11-196118.2 |
| chr9_121324567_A_G | 9 | 121324567 | 2.62E-08 | TPT1P9,RP11-349E4.1 |
| rs1889749 | 9 | 133958298 | 4.76E-08 | LAMC3 |
| chr9_138983799_A_G | 9 | 138983799 | 1.16E-09 | NACC2 |
| rs3739467 | 9 | 139009107 | 3.46E-08 | C9orf69 |
| chr10_5120332_G_T | 10 | 5120332 | 4.02E-12 | AKR1C3 |
| chr10_23728059_A_G | 10 | 23728059 | 1.55E-12 | snoU13,OTUD1 |
| chr10_43692630_C_T | 10 | 43692630 | 7.76E-08 | RASGEF1A |
| chr10_80842827_A_C | 10 | 80842827 | 1.83E-12 | ZMIZ1 |
| chr10_81006391_C_T | 10 | 81006391 | 1.97E-09 | ZMIZ1 |
| chr10_82842595_A_G | 10 | 82842595 | 3.77E-09 | WARS2P1,RPA2P2 |
| corect_rs139699745 | 10 | 87470588 | 8.60E-09 | GRID1 |
| rs4494234 | 10 | 89417840 | 2.64E-09 | RP11-57C13.3 |
| chr11_871530_C_T | 11 | 871530 | 1.74E-10 | CHID1 |
| exm876085 | 11 | 1267727 | 1.84E-09 | NA |
| rs3858522 | 11 | 2057647 | 9.06E-22 | H19,IGF2 |
| rs12807478 | 11 | 2161955 | 2.71E-10 | IGF2 |
| chr11_2597984_A_G | 11 | 2597984 | 1.41E-16 | KCNQ1 |
| exm889520 | 11 | 9074651 | 2.37E-08 | NA |
| chr11_32938165_A_G | 11 | 32938165 | 1.83E-10 | QSER1 |
| rs12289759 | 11 | 49095165 | 1.27E-08 | CTD-2132H18.3 ,UBTFL7 |
| chr11_59071087_C_T | 11 | 59071087 | 1.02E-10 | RN7SL435P,OR5AN2P |
| chr11_65398096_C_T | 11 | 65398096 | 4.64E-08 | PCNXL3 |
| chr11_65659450_A_G | 11 | 65659450 | 3.45E-08 | CCDC85B,FOSL1 |
| rs557625 | 11 | 68634722 | 2.75E-08 | CPT1A,RP11-757G1.6 |
| chr11_68980828_G_T | 11 | 68980828 | 1.92E-14 | RP11-554A11.8,MYEOV |
| chr11_69459104_C_G | 11 | 69459104 | 3.31E-08 | CCND1 |
| chr11_111757486_A_ G | 11 | 111757486 | 5.05E-22 | C11orf1,RPL37AP8 |
| chr12_28530125_C_G | 12 | 28530125 | 2.12E-15 | CCDC91 |
| rs7959675 | 12 | 39520651 | 1.78E-27 | RP11-554L12.1,RP11-421H10.2 |
| chr12_49951528_C_T | 12 | 49951528 | 3.61E-08 | KCNH3,MCRS1 |
| rs4135136 | 12 | 104379994 | 1.07E-08 | TDG |
| rs9658256 | 12 | 117799549 | 4.44E-08 | NOS1 |
| chr12_133155025_C_ T | 12 | 133155025 | 7.84E-08 | FBRSL1 |
| chr13_27131826_G_T | 13 | 27131826 | 7.15E-08 | CDK8,WASF3 |
| chr13_32914904_G_T | 13 | 32914904 | 2.11E-08 | BRCA2 |
| corect_rs111968842 | 13 | 46603855 | 9.27E-19 | ZC3H13 |
| chr13_113284191_C_ T | 13 | 113284191 | 4.62E-11 | TUBGCP3,C13orf35 |
| chr14_21816052_C_T | 14 | 21816052 | 2.71E-13 | RPGRIP1 |
| rs7144699 | 14 | 33250907 | 2.42E-08 | AKAP6 |
| rs79214033 | 14 | 54461711 | 6.14E-09 | ATP5C1P1,CDKN3 |
| chr14_76886176_C_T | 14 | 76886176 | 1.74E-14 | ESRRB |
| rs7158184 | 14 | 92586247 | 5.25E-12 | NDUFB1 |
| chr14_101121371_G_ T | 14 | 101121371 | 3.13E-13 | CTD-2644121.1,LINC00523 |
| chr14_104819550_C_ T | 14 | 104819550 | 8.40E-12 | RP11-260M19.2,RP11-260M19.1 |
| chr14_105240784_C_ G | 14 | 105240784 | 1.22E-08 | AKT1 |
| rs12910968 | 15 | 26849256 | 1.24E-08 | GABRB3 |
| chr15_40529113_A_G | 15 | 40529113 | 8.44E-08 | PAK6 |
| rs2903992 | 15 | 78709146 | 5.34E-08 | RP11-5023.1,IREB2 |
| chr16_611683_C_T | 16 | 611683 | 5.80E-09 | C16orf11 |
| exm1197778 | 16 | 711429 | 8.92E-08 | NA |
| chr16_8755147_C_T | 16 | 8755147 | 2.68E-14 | METTL22,ABAT |
| chr16_52328666_C_T | 16 | 52328666 | 9.47E-12 | RP11-142G’,TOX3 |
| rs71647871 | 16 | 55857570 | 3.39E-11 | CES1 |
| chr16_61365835_C_T | 16 | 61365835 | 3.43E-08 | RP11-5106.1,CDH8 |
| rs12447656 | 16 | 77749442 | 2.95E-11 | AC092724.1,NUDT7 |
| rs2326255 | 16 | 84435229 | 3.39E-09 | ATP2C2 |
| chr16_88835229_C_T | 16 | 88835229 | 1.25E-21 | PIEZO1 |
| rs1968109 | 16 | 89854829 | 3.43E-09 | FANCA |
| chr17_7164499_C_T | 17 | 7164499 | 6.92E-13 | CLDN7 |
| chr17_29055710_C_T | 17 | 29055710 | 4.24E-08 | SUZ 12P |
| rs9910757 | 17 | 29839696 | 3.74E-16 | RAB11FIP4 |
| chr17_35438073_C_T | 17 | 35438073 | 3.92E-08 | AATF,ACACA |
| chr17_41196821_IND EL_T | 17 | 41196821 | 4.39E-38 | BRCA1 |
| chr17_46041404_A_T | 17 | 46041404 | 9.29E-10 | RP11-6N17.9 |
| chr17_77945111_C_T | 17 | 77945111 | 1.92E-09 | TBC1D16 |
| chr17_78243909_A_G | 17 | 78243909 | 2.00E-16 | RNF213 |
| corect_rs117045048 | 17 | 78927335 | 3.90E-10 | RPTOR |
| rs62078752 | 17 | 80057953 | 5.83E-09 | FASN,CCDC57 |
| rs292347 | 18 | 5132226 | 4.56E-09 | RP11-92G19.2,RP11-190I17.4 |
| rs8087677 | 18 | 74282273 | 1.83E-08 | NA |
| chr18_74757361_C_T | 18 | 74757361 | 1.78E-08 | MBP |
| rs61744452 | 19 | 1003657 | 4.10E-08 | GRIN3B |
| chr19_2090950_C_T | 19 | 2090950 | 3.07E-10 | MOB3A |
| chr19_2472833_C_T | 19 | 2472833 | 1.06E-16 | AC005624.2,GADD45B |
| chr19_13269181_A_G | 19 | 13269181 | 2.08E-08 | CTC-250I14.1 |
| rs13345139 | 19 | 17435033 | 6.47E-08 | ANO8 |
| chr19_19548246_A_G | 19 | 19548246 | 5.99E-08 | GATAD2A |
| chr19_28927856_C_T | 19 | 28927856 | 1.23E-10 | AC005307.3 |
| rs73022296 | 19 | 33774236 | 2.39E-08 | SLC7A10,CTD-2540B15.12 |
| chr19_42463049_IND EL_T | 19 | 42463049 | 1.62E-10 | RABAC1 |
| rs2974217 | 19 | 48087491 | 1.20E-09 | RN7SL322P,CTD-2571L23.8 |
| chr19_51302154_C_T | 19 | 51302154 | 3.38E-12 | C19orf48 |
| chr19_54502409_C_T | 19 | 54502409 | 7.08E-16 | CACNG6 |
| rs62126247 | 19 | 58165417 | 2.81E-10 | ZNF211,AC003682.17 |
| chr20_25058424_G_T | 20 | 25058424 | 4.04E-11 | VSX1 |
| chr20_36836192_A_G | 20 | 36836192 | 5.69E-13 | TGM2,KIAA1755 |
| rs2427282 | 20 | 60892545 | 6.82E-08 | LAMA5 |
| chr20_61052092_C_T | 20 | 61052092 | 5.29E-08 | GATA5,RP13-379O24.3 |
| rs41309371 | 20 | 61443716 | 2.42E-08 | OGFR |
| chr20_62321128_A_G | 20 | 62321128 | 3.99E-12 | RTEL1 |
| chr20_62328445_AC AACCGTG_INDEL | 20 | 62328445 | 5.54E-08 | TNTRSF6B |
| chr21_19567725_C_T | 21 | 19567725 | 6.90E-12 | CHODL |
| chr21_41532756_C_T | 21 | 41532756 | 9.56E-10 | DSCAM |
| chr21_46408134_A_G | 21 | 46408134 | 1.90E-08 | FAM207A,LINC00163 |
| chr22_17733251_A_G | 22 | 17733251 | 6.39E-08 | CECR1,CECR3 |
| rs450710 | 22 | 21446768 | 3.57E-10 | TUBA3GP,BCRP2 |
| chr22_23919448_C_T | 22 | 23919448 | 1.75E-08 | IGLL1 |
| rs4820792 | 22 | 29161007 | 5.25E-08 | HSCB,CCDC117 |
| rs1971653 | 22 | 31023326 | 7.53E-09 | TCN2, SLC35E4 |
| chr22_37686987_G_T | 22 | 37686987 | 1.08E-11 | CYTH4 |
| chr22_50436488_A_G | 22 | 50436488 | 1.22E-08 | IL17REL |
| corect rs181578054 | 22 | 51084318 | 9.72E-12 | ARSA,Y_RNA |
| kgp22771613 | 23 | 43639615 | 5.49E-11 | NA |
| rs16988375 | 23 | 91340786 | 8.93E-08 | PCDH11X |
| High-genetic-risk case sub-population vs. the control population | | | | |
| SNP | Chr. | Position | p.value | Genes* |
| chr1_1914124_C_T | 1 | 1914124 | 1.73E-09 | C1orf222 |
| chr1_2501064_A_G | 1 | 2501064 | 7.54E-08 | RP3-395M20.7 |
| rs74820022 | 1 | 3408706 | 2.18E-08 | MEGF6 |
| chr1_10617906_A_T | 1 | 10617906 | 9.34E-10 | PEX14 |
| chr1_15348453_A_C | 1 | 15348453 | 1.39E-11 | KAZN |
| rs602946 | 1 | 20915535 | 1.74E-09 | CDA |
| chr1_28632870_A_C | 1 | 28632870 | 9.41E-08 | SESN2,MED 18 |
| rs4316319 | 1 | 78810600 | 5.04E-08 | PTGFR,RP11-183M13.1 |
| chr1_97788840_A_G | 1 | 97788840 | 5.53E-15 | DPYD |
| rs1884296 | 1 | 115235716 | 3.41E-10 | AMPD1 |
| chr1_171056203_C_T | 1 | 171056203 | 2.74E-19 | RP5-1092L12.2,FMO3 |
| rs10752892 | 1 | 183036055 | 1.62E-09 | LAMC 1 |
| chr1_202172594_C_T | 1 | 202172594 | 4.36E-08 | LGR6 |
| chr1_204008939_C_T | 1 | 204008939 | 1.90E-08 | LINC00303 |
| rs729125 | 1 | 238118749 | 7.84E-16 | MTND5P18,YWHAQP9 |
| corect_rs189944458 | 2 | 18059890 | 4.46E-08 | KCNS3 |
| chr2_23168305_A_G | 2 | 23168305 | 7.95E-10 | RN7SKP27,AC016768.1 |
| chr2_23222481_C_T | 2 | 23222481 | 9.51E-17 | RN7SKP27,AC016768.1 |
| chr2_26526169_A_G | 2 | 26526169 | 2.19E-08 | HADHB,GPR113 |
| chr2_28150862_A_C | 2 | 28150862 | 2.15E-34 | BRE,MRPL33 |
| chr2_29009089_A_C | 2 | 29009089 | 2.15E-11 | PPP1CB,SPDYA |
| rs6707103 | 2 | 103994511 | 9.01E-08 | AC073987.2,AC092568.1 |
| chr2_111862303_C_T | 2 | 111862303 | 3.44E-20 | AC096670.3 |
| rs11678485 | 2 | 143788391 | 1.50E-08 | KYNU |
| chr2_150721127_A_C | 2 | 150721127 | 2.97E-08 | AC007364.1,AC016682.1 |
| chr2_172017549_G_T | 2 | 172017549 | 3.90E-08 | TLK1 |
| rs74948099 | 2 | 231010534 | 2.85E-09 | AC009950.2 |
| corect_rs187745955 | 3 | 20612509 | 9.17E-10 | RNU6-815P,AC104441.1 |
| rs9851291 | 3 | 20994957 | 5.72E-14 | RNU6-815P,AC104441.1 |
| rs2343912 | 3 | 32445089 | 6.86E-10 | CMTM7 |
| rs9813107 | 3 | 40987921 | 1.79E-09 | RP11-761N21.1,RP11-520A21.1 |
| chr3_46742523_A_C | 3 | 46742523 | 5.01E-22 | ALS2CL,TMIE |
| chr3_49501384_C_T | 3 | 49501384 | 5.43E-09 | NICN1,RNA5SP130 |
| chr3_50192826_C_T | 3 | 50192826 | 1.16E-14 | SEMA3F |
| chr3_53880367_G_T | 3 | 53880367 | 5.42E-11 | CHDH |
| rs13098429 | 3 | 114875160 | 1.01E-08 | ZBTB20,RP11-190P13.2 |
| chr3_138459216_A_G | 3 | 138459216 | 1.09E-10 | PIK3CB |
| rs 11925421 | 3 | 145888162 | 2.47E-10 | PLOD2,PLSCR4 |
| chr3_149390610_A_T | 3 | 149390610 | 1.02E-14 | WWTR1 |
| chr3_149688990_A_G | 3 | 149688990 | 4.65E-09 | PFN2 |
| rs77204838 | 4 | 8605475 | 1.37E-13 | CPZ,GPR78 |
| chr4_9462484_G_T | 4 | 9462484 | 7.60E-14 | OR7E86P,OR7E84P |
| chr4_39691575_G_T | 4 | 39691575 | 4.34E-08 | RP11-539G18.2,UBE2K |
| rs11735107 | 4 | 40038146 | 1.41E-10 | KRT18P25,RP11-333E13.4 |
| kgp21013528 | 4 | 46152421 | 1.44E-08 | NA |
| rs10518461 | 4 | 126164298 | 3.32E-11 | ANKRD50,FAT4 |
| rs73859240 | 4 | 162446738 | 7.89E-09 | FSTL5 |
| corect_rs112923443 | 4 | 172579034 | 4.05E-09 | RP 11-97E7.2,GALNTL6 |
| chr5_521096_C_T | 5 | 521096 | 2.61E-12 | SLC9A3 |
| chr5_770093_A_G | 5 | 770093 | 3.33E-10 | ZDHHC11 |
| rs456752 | 5 | 1484826 | 8.79E-08 | LPCAT1 |
| chr5_26168640_C_T | 5 | 26168640 | 1.01E-20 | RNU4-43P,RP11-351N6.1 |
| chr5_49502516_C_T | 5 | 49502516 | 3.71E-10 | CTD-2013M15.1,EMB |
| chr5_67103091_A_C | 5 | 67103091 | 1.17E-09 | RP11-434D9.2 |
| rs554514 | 5 | 111130274 | 3.46E-20 | NREP |
| chr5_116877991_A_C | 5 | 116877991 | 5.60E-16 | LINC00992 |
| rs801752 | 5 | 134819978 | 1.49E-15 | CTB-138E5.1, NEUROG1 |
| rs1990941 | 5 | 164991054 | 3.85E-10 | CTC-535M15.2 |
| rs1736999 | 6 | 29764656 | 2.40E-11 | HLA-V |
| rs1633097 | 6 | 29784192 | 9.77E-08 | MICG,HLA-G |
| chr6_30243235_C_T | 6 | 30243235 | 8.17E-10 | HCG17,HLA-L |
| rs130065 | 6 | 31122500 | 7.11E-20 | CCHCR1 |
| chr6_31161571_C_G | 6 | 31161571 | 5.62E-08 | POU5F1,XXbac-BPG299F13.17 |
| chr6_32470283_A_C | 6 | 32470283 | 9.64E-09 | HLA-DRB9,HLA-DRB5 |
| chr6_32480507_C_T | 6 | 32480507 | 2.91E-18 | HLA-DRB9,HLA-DRB5 |
| chr6_32484554_A_T | 6 | 32484554 | 1.09E-10 | HLA-DRB9,HLA-DRB5 |
| chr6_32552168_A_G | 6 | 32552168 | 9.24E-13 | HLA-DRB1 |
| rs9271611 | 6 | 32591609 | 1.23E-11 | HLA-DRB1,HLA-DQA1 |
| chr6_32691173_C_T | 6 | 32691173 | 9.40E-10 | XXbac-BPG254F23.7,HLA-DQB3 |
| rs9275851 | 6 | 32691186 | 2.29E-18 | XXbac-BPG254F23.7,HLA-DQB3 |
| rs7753169 | 6 | 36614326 | 2.28E-24 | RNU1-88P,Y_RNA |
| chr6_75480993_GT_I NDEL | 6 | 75480993 | 3.05E-10 | RP11-554D15.3,RP11-560O20.1 |
| chr6_117598048_A_G | 6 | 117598048 | 9.39E-08 | VGLL2,ROS1 |
| rs4895919 | 6 | 131630319 | 3.41E-09 | AKAP7,RPL21P67 |
| rs79830246 | 6 | 151380101 | 7.30E-12 | MTHFD1L |
| chr6_153077792_A_G | 6 | 153077792 | 5.24E-10 | VIP |
| chr6_161398697_G_I NDEL | 6 | 161398697 | 5.48E-09 | RP3-428L16.1,RP3-428L16.2 |
| rs61729932 | 7 | 2577816 | 2.60E-08 | BRAT1 |
| chr7_4832371_A_G | 7 | 4832371 | 4.54E-09 | AP5Z1 |
| corect_rs117345894 | 7 | 5470678 | 7.06E-08 | RP11-1275H24.3,FBXL18 |
| rs28379235 | 7 | 16129153 | 2.80E-15 | AC006035.1,RP11-196O16.1 |
| rs4724080 | 7 | 41949635 | 9.16E-09 | INHBA-AS,1GLI3 |
| rs4717142 | 7 | 74027839 | 3.35E-08 | RP5-1186P10.2,GTF2I |
| chr7_91782274_A_G | 7 | 91782274 | 8.66E-09 | CTB-161K23.1,LRRD1 |
| rs58348977 | 7 | 99188014 | 2.08E-08 | GS1-259H13.10 |
| chr7_118831547_C_T | 7 | 118831547 | 9.99E-32 | RP11-500M10.1,AC091320.2 |
| rs10464695 | 7 | 128766463 | 5.19E-08 | CYCSP20,RP11-286H14.4 |
| rs62489409 | 7 | 129632800 | 3.00E-34 | RP11-306G20.1 |
| rs3927319 | 7 | 141248158 | 6.17E-08 | RP11-744I24.2 |
| rs7821602 | 8 | 5220317 | 1.50E-09 | RN7SL318P,RP11-745K9.2 |
| chr8_17265628_A_G | 8 | 17265628 | 2.09E-09 | MTMR7 |
| chr8_21408145_C_T | 8 | 21408145 | 4.41E-08 | AC022716.1,GFRA2 |
| chr8_87816647_C_T | 8 | 87816647 | 2.08E-09 | RP11-386D6.2 |
| chr8_128146308_G_T | 8 | 128146308 | 5.07E-10 | PRNCR1,CASC19 |
| rs7856798 | 9 | 5952026 | 1.48E-08 | KIAA2026 |
| rs2578291 | 9 | 6642973 | 5.41E-08 | GLDC |
| rs1180130 | 9 | 72904219 | 7.71E-09 | SMC5 |
| rs59032320 | 9 | 109874246 | 6.46E-08 | RP11-508N12.2,RP11-196118.2 |
| chr10_5120332_G_T | 10 | 5120332 | 1.15E-14 | AKR1C3 |
| rs2388742 | 10 | 8532669 | 2.67E-09 | RP11-543F8.3,KRT8P37 |
| chr10_23728059_A_G | 10 | 23728059 | 1.32E-09 | snoU13,OTUD1 |
| chr10_80842827_A_C | 10 | 80842827 | 2.61E-14 | ZMIZ1 |
| chr10_82842595_A_G | 10 | 82842595 | 1.04E-08 | WARS2P 1,RPA2P2 |
| rs11200014 | 10 | 123334930 | 8.61E-09 | FGFR2 |
| chr10_123337066_C_ T | 10 | 123337066 | 1.91E-09 | FGFR2 |
| rs2912780 | 10 | 123337117 | 2.99E-08 | FGFR2 |
| rs2912779 | 10 | 123337182 | 5.95E-08 | FGFR2 |
| rs2981579 | 10 | 123337335 | 3.99E-08 | FGFR2 |
| rs1078806 | 10 | 123338975 | 4.14E-09 | FGFR2 |
| rs11599804 | 10 | 123340664 | 1.51E-09 | FGFR2 |
| rs4752571 | 10 | 123342567 | 1.42E-09 | FGFR2 |
| rs1219651 | 10 | 123344501 | 7.79E-10 | FGFR2 |
| rs2981575 | 10 | 123346116 | 7.56E-09 | FGFR2 |
| rs1219648 | 10 | 123346190 | 1.58E-09 | FGFR2 |
| rs1219642 | 10 | 123348389 | 1.12E-09 | FGFR2 |
| rs2912774 | 10 | 123348662 | 1.61E-08 | FGFR2 |
| rs2936870 | 10 | 123348902 | 1.28E-08 | FGFR2 |
| rs2981584 | 10 | 123350216 | 6.15E-09 | FGFR2 |
| rs2860197 | 10 | 123351302 | 8.13E-08 | FGFR2 |
| rs2420946 | 10 | 123351324 | 9.85E-08 | FGFR2 |
| rs2981582 | 10 | 123352317 | 3.70E-08 | FGFR2 |
| rs3135718 | 10 | 123353869 | 8.56E-08 | FGFR2 |
| chr11_871530_C_T | 11 | 871530 | 7.51E-09 | CHID1 |
| exm876085 | 11 | 1267727 | 7.73E-08 | NA |
| rs3858522 | 11 | 2057647 | 2.21E-16 | H19,IGF2 |
| chr11_2597984_A_G | 11 | 2597984 | 1.97E-15 | KCNQ1 |
| chr11_32938165_A_G | 11 | 32938165 | 7.10E-11 | QSER1 |
| rs12289759 | 11 | 49095165 | 6.84E-10 | CTD-2132H18.3,UBTFL7 |
| chr11_59071087_C_T | 11 | 59071087 | 3.24E-11 | RN7SL435P,OR5AN2P |
| chr11_68980828_G_T | 11 | 68980828 | 3.53E-12 | RP11-554A11.8,MYEOV |
| chr11_111757486 _A_G | 11 | 111757486 | 4.40E-23 | C11orf1,RPL37AP8 |
| chr11_130943681_A_G | 11 | 130943681 | 9.40E-16 | RN7SL167P,AP002806.1 |
| kgp18707282 | 12 | 21527350 | 3.54E-08 | NA |
| chr12_28530125_C_G | 12 | 28530125 | 2.79E-15 | CCDC91 |
| rs7959675 | 12 | 39520651 | 1.51E-28 | RP11-554L12.1,RP11-421H10.2 |
| rs9658256 | 12 | 117799549 | 2.25E-08 | NOS 1 |
| corect_rs11968842 | 13 | 46603855 | 1.11E-16 | ZC3H13 |
| chr13_113284191_C_T | 13 | 113284191 | 3.68E-13 | TUBGCP3,C13orf35 |
| chr14_21816052_C_T | 14 | 21816052 | 2.67E-11 | RPGRIP1 |
| chr14_76886176_C_T | 14 | 76886176 | 1.12E-13 | ESRRB |
| rs7158184 | 14 | 92586247 | 1.72E-13 | NDUFB1 |
| chr14_101121371_G_T | 14 | 101121371 | 1.70E-11 | CTD-2644I2 1.1,LINC00523 |
| chr14_104819550_C_T | 14 | 104819550 | 6.64E-11 | RP11-26OM19.2,RP11-260M19.1 |
| rs2903992 | 15 | 78709146 | 6.81E-08 | RP11-5O23.1,IREB2 |
| chr16_8755147_C_T | 16 | 8755147 | 4.21E-12 | METTL22,ABAT |
| chr16_52328666_C_T | 16 | 52328666 | 3.22E-13 | RP11-142G1.2,TOX3 |
| chr16_52583143_C_T | 16 | 52583143 | 2.31E-10 | TOX3,CASC16 |
| rs71647871 | 16 | 55857570 | 1.85E-10 | CES1 |
| rs12447656 | 16 | 77749442 | 7.75E-08 | AC092724.1,NUDT7 |
| rs2326255 | 16 | 84435229 | 9.20E-11 | ATP2C2 |
| chr16_88835229_C_T | 16 | 88835229 | 2.42E-17 | PIEZO1 |
| rs1968109 | 16 | 89854829 | 3.46E-09 | FANCA |
| chr17_7164499_C_T | 17 | 7164499 | 3.39E-13 | CLDN7,RP1-4G17.5 |
| chr17_29055710_C_T | 17 | 29055710 | 2.18E-09 | SUZ12P |
| rs9910757 | 17 | 29839696 | 5.35E-11 | RAB 1 1FIP4 |
| chr17_41196821_IND EL_T | 17 | 41196821 | 8.17E-35 | BRCA1 |
| chr17_46041404_A_T | 17 | 46041404 | 4.91E-10 | RP11-6N17.9 |
| corect_rs116995945 | 17 | 55095153 | 7.01E-08 | SCPEP1 ,RNF126P1 |
| chr17_77945111_C_T | 17 | 77945111 | 8.01E-08 | TBC1D16 |
| chr17_78243909_A_G | 17 | 78243909 | 1.68E-12 | RNF213 |
| corect_rs117045048 | 17 | 78927335 | 4.66E-09 | RPTOR |
| rs292347 | 18 | 5132226 | 8.49E-12 | RP11-92G19.2,RP11-190I17.4 |
| chr19_2090950_C_T | 19 | 2090950 | 7.03E-09 | MOB3A |
| chr19_2472833_C_T | 19 | 2472833 | 9.54E-12 | AC005624.2,GADD45B |
| rs34923393 | 19 | 15756618 | 2.00E-08 | CYP4F3 |
| chr19_19548246_A_G | 19 | 19548246 | 1.85E-08 | GATAD2A |
| chr19_28927856_C_T | 19 | 28927856 | 9.99E-08 | AC005307.3 |
| rs2974217 | 19 | 48087491 | 9.03E-09 | RN7SL322P,CTD-2571L23.8 |
| chr19_51302154_C_T | 19 | 51302154 | 7.91E-09 | C19orf48 |
| chr19_54502409_C_T | 19 | 54502409 | 8.63E-13 | CACNG6 |
| rs62126247 | 19 | 58165417 | 5.06E-08 | ZNF211,AC003682.17 |
| chr20_25058424_G_T | 20 | 25058424 | 2.28E-09 | VSX 1 |
| chr20_36836192_A_G | 20 | 36836192 | 4.03E-10 | TGM2,KIAA1755 |
| chr20_62321128_A_G | 20 | 62321128 | 1.03E-09 | RTEL1 |
| chr21_19567725_C_T | 21 | 19567725 | 1.80E-11 | CHODL |
| chr21_41532756_C_T | 21 | 41532756 | 6.41E-13 | DSCAM |
| chr22_17733251_A_G | 22 | 17733251 | 4.22E-08 | CECR1,CECR3 |
| rs450710 | 22 | 21446768 | 8.30E-09 | TUBA3GP,BCRP2 |
| rs2527343 | 22 | 30111558 | 2.01E-08 | RP1-76B20.11 |
| chr22_37686987_G_T | 22 | 37686987 | 1.30E-11 | CYTH4 |
| corect rs181578054 | 22 | 51084318 | 8.67E-11 | ARSA,Y RNA |
| kgp22771613 | 23 | 43639615 | 1.18E-08 | NA |
| Normal-genetic-risk case sub-population vs. the control population | | | | |
| SNP | Chromosome | Position | p.value | Genes |

None
*Genes are annotate as overlapped gene or nearest upstream/downstream gene for each SNP

TABLE 6

Pathway enrichment analysis of genes associated with the 149 shared significant SNPs

| Pathway ID | Description | Parent(s) | p-Value | Genes Involved | SNPs |
|---|---|---|---|---|---|
| R-HSA-69473 | G2/M DNA damage checkpoint | Cell Cycle | 0.038752 | BRE,BRCA1 | chr17_41196821_IN DEL_T,chr2_28150862_A_C |
| R-HSA-376172 | DSCAM interactions | Developmental Biology | 0.043704 | DSCAM | chr21_41532756_C_T |
| R-HSA-9635465 | Suppression of apoptosis | Disease | 0.028032 | RNF213 | chr17_78243909_A_G |
| R-HSA-9673767 | Signaling by PDGFRA transmembrane, juxtamembrane and kinase domain mutants | Disease | 0.047584 | PIK3CB | chr3_138459216_A_G |
| R-HSA-9673770 | Signaling by PDGFRA extracellular domain mutants | Disease | 0.047584 | PIK3CB | chr3_138459216_A_G |
| R-HSA-5693554 | Resolution of D-loop Structures through Synthesis-Dependent Strand Annealing (SDSA) | DNA Repair | 0.004901 | BRCA1,RTEL1 | chr17_41196821_IN DEL_T,chr20_62321 128_A_G |
| R-HSA-5693537 | Resolution of D-Loop Structures | DNA Repair | 0.008289 | BRCA1,RTEL1 | chr17_41196821_IN DEL_T,chr20_62321 128_A_G |
| R-HSA-5693567 | HDR through Homologous Recombination (HRR) or Single Strand Annealing (SSA) | DNA Repair | 0.010909 | BRE,BRCA1,RTEL1 | chr17_41196821_IN DEL_T,chr20_62321 128_A_G,chr2_2815 0862_A_C |
| R-HSA-5693538 | Homology Directed Repair | DNA Repair | 0.012529 | BRE,BRCA1,RTEL1 | chr17_41196821_IN DEL_T,chr20_62321 128_A_G,chr2_2815 0862_A_C |
| R-HSA-5693571 | Nonhomologous End-Joining (NHEJ) | DNA Repair | 0.018038 | BRE,BRCA1 | chr17_41196821_IN DEL_T,chr2_281508 62_A_C |
| R-HSA-5693532 | DNA Double-Strand Break Repair | DNA Repair | 0.021846 | BRE,BRCA1,RTEL1 | chr17_41196821_IN DEL_T,chr20_62321 128_A_G,chr2_2815 0862_A_C |
| R-HSA-5693565 | Recruitment and ATM-mediated phosphorylation | DNA Repair | 0.022973 | BRE,BRCA1 | chr17_41196821_IN DEL_T,chr2281508 62_A_C |
| | of repair and signaling proteins at DNA double strand breaks | | | | |
| R-HSA-5693606 | DNA Double Strand Break Response | DNA Repair | 0.023719 | BRE,BRCA1 | chr17_41196821_IN DEL_T,chr2_281508 62_A_C |
| R-HSA-5685942 | HDR through Homologous Recombination (HRR) | DNA Repair | 0.030034 | BRCA1,RTEL1 | chr17_41196821_IN DEL_T,chr20_62321 128_A_G |
| R-HSA-73894 | DNA Repair | DNA Repair | 0.035376 | BRE,BRCA1,RTEL1,FANCA | chr17_41196821_IN DEL_T,chr20_62321 128_A_G,chr2_2815 0862_A_C,rs1968109 |
| R-HSA-5693607 | Processing of DNA double-strand break ends | DNA Repair | 0.041535 | BRE,BRCA1 | chr17_41196821_IN DEL_T,chr2_281508 62_A_C |
| R-HSA-8951671 | RUNX3 regulates YAP1-mediated transcription | Gene expression (Transcription) | 0.031973 | WWTR1 | chr3_149390610_A_T |
| R-HSA-8956321 | Nucleotide salvage | Metabolism | 0.003845 | AMPD1,CDA | rs1884296,rs602946 |
| R-HSA-1430728 | Metabolism | Metabolism | 0.006471 | LPCAT1,MT MR7,CHDH, GLDC,MTHF DIL,AKR1C 3,ACOXL,PP P1CB,RTEL1 ,AMPD1,KC NS3,PIK3CB, CES1,CDA,D PYD,NDUFB1 | chr10_5120332_G_T ,chr1_97788840_A_ G,chr20_62321128_ A_G,chr2_1186230 3_C_T,chr2_2900908 9_A_C,chr3_138459 216_A_G,chr3_5388 0367_G_T,chr8_172 65628_A_G,rs18842 96,rs189944458,rs25 78291,rs456752,rs60 2946,rs7158184,rs71 647871,rs79830246 |
| R-HSA-15869 | Metabolism of nucleotides | Metabolism | 0.007216 | AMPD1,CDA ,DPYD | ch1_97788840_A_G ,rs1884296,rs602946 |
| R-HSA-6783984 | Glycine degradation | Metabolism | 0.016114 | GLDC | rs2578291 |
| R-HSA-6798163 | Choline catabolism | Metabolism | 0.024075 | CHDH | chr3_53880367_G_T |
| R-HSA-389887 | Beta-oxidation of pristanoyl- CoA | Metabolism | 0.035899 | ACOXL | chr2_111862303_C_T |
| R-HSA-1483255 | PI Metabolism | Metabolism | 0.042479 | MTMR7,PIK 3CB | chr3_138459216_A_ G,chr8_17265628_A G |
| R-HSA-1660517 | Synthesis of PIPs at the late endosome membrane | Metabolism | 0.043704 | MTMR7 | chr8_17265628_A_G |
| R-HSA-73614 | Pyrimidine salvage | Metabolism | 0.043704 | CDA | rs602946 |
| R-HSA-73621 | Pyrimidine catabolism | Metabolism | 0.047584 | DPYD | chr1_97788840_A_G |
| R-HSA-5689901 | Metall oprotease DUBs | Metabolism of proteins | 0.006076 | BRE,BRCA1 | chr17_41196821_IN DEL_T,chr2_281508 62 A_C |
| R-HSA-3108214 | SUMOylation of DNA damage response and repair proteins | Metabolism of proteins | 0.036939 | SMC5,BRCA1 | chr17_41196821_IN DEL_T,rs1180130 |
| R-HSA-5576891 | Cardiac conduction | Muscle contraction | 0.002163 | WWTR1,NOS1,KCNQ1,CACNG6 | chr11_2597984_A_G,chr19_54502409_C_T,chr3_149390610_A_T,rs9658256 |
| R-HSA-5576893 | Phase 2 - plateau phase | Muscle contraction | 0.004536 | KCNQ1,CACNG6 | chr11_2597984_A _G ,chr19_54502409_C_T |
| R-HSA-397014 | Muscle contraction | Muscle contraction | 0.009114 | WWTR1,NOS1,KCNQ1,CACNG6 | chr11_2597984_A_G ,chr19_54502409_C T,chr3_149390610_ A_T,rs9658256 |
| R-HSA-5576890 | Phase 3 - rapid repolarisation | Muscle contraction | 0.031973 | KCNQ1 | chr11_2597984_A_G |
| R-HSA-5578768 | Physiological factors | Muscle contraction | 0.047584 | WWTR1 | chr3_149390610_A_T |
| R-HSA-1296072 | Voltage gated Potassium channels | Neuronal System | 0.013039 | KCNQ1,KCNS3 | chr11_2597984 _A _G ,rs189944458 |
| R-HSA-8943724 | Regulation of PTEN gene transcription | Signal Transduction | 0.02524 | GATAD2A,RPTOR | chr19_19548246 _A_G,rs1 17045048 |
| R-HSA-170834 | Signaling by TGF-beta Receptor Complex | Signal Transduction | 0.03516 | WWTR1,PPP1CB | chr2_29009089_A_C ,chr3 149390610 AT |
| R-HSA-198203 | PI3K/AKT activation | Signal Transduction | 0.035899 | PIK3CB | chr3 _138459216 _ A_G |
| R-HSA-391908 | Prostanoid ligand receptors | Signal Transduction | 0.035899 | PTGFR | rs4316319 |
| R-HSA-9027276 | Erythropoietin activates Phosphoinositide -3-kinase (PI3K) | Signal Transduction | 0.047584 | PIK3CB | chr3_138459216_A_G |
| R-HSA-425986 | Sodium/Proton exchangers | Transport of small molecules | 0.035899 | SLC9A3 | chr5_521096_C_T |

### Example 4 - Interpretation of the DNN Model

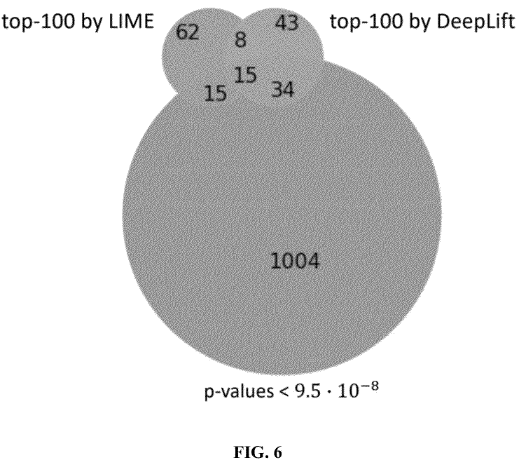

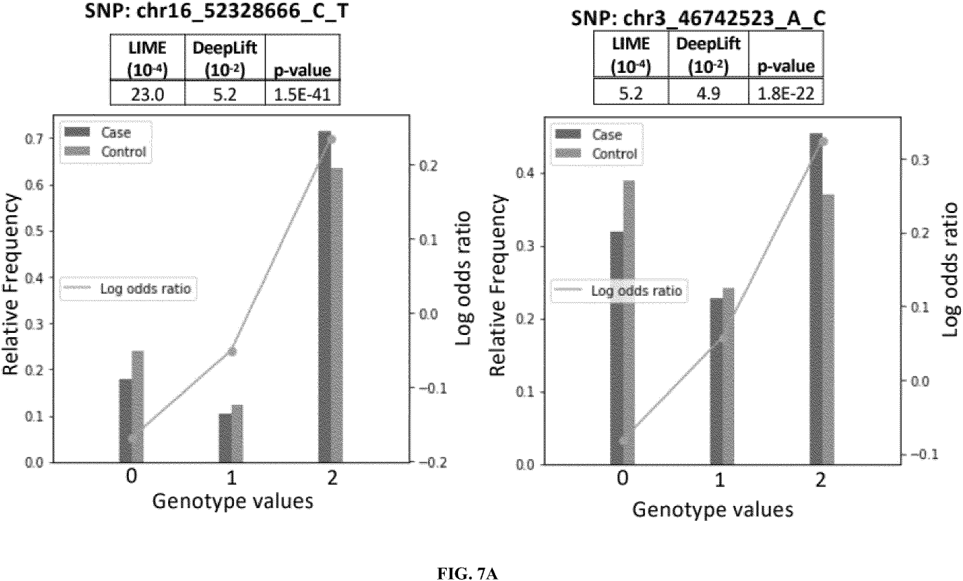

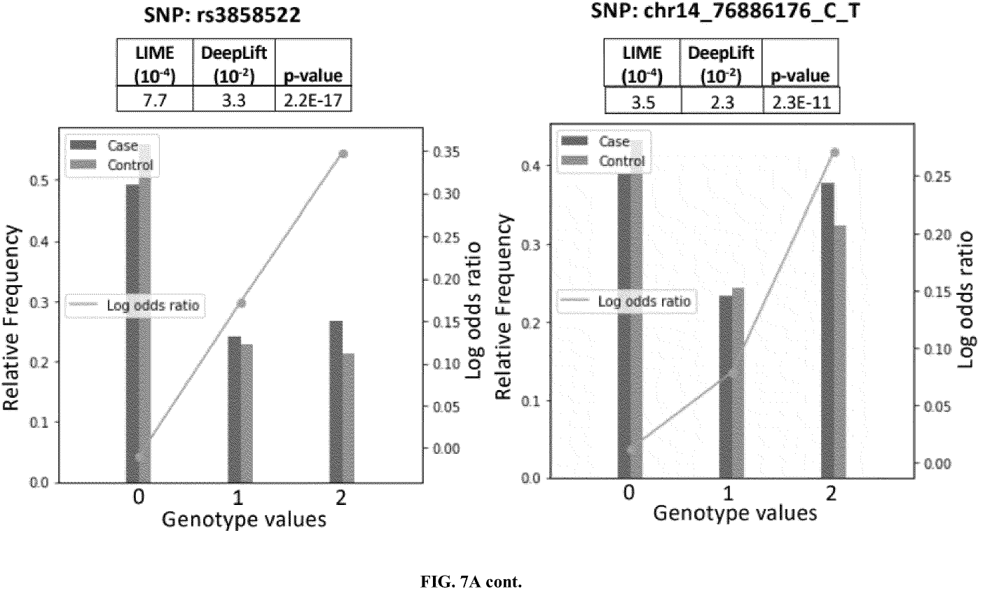

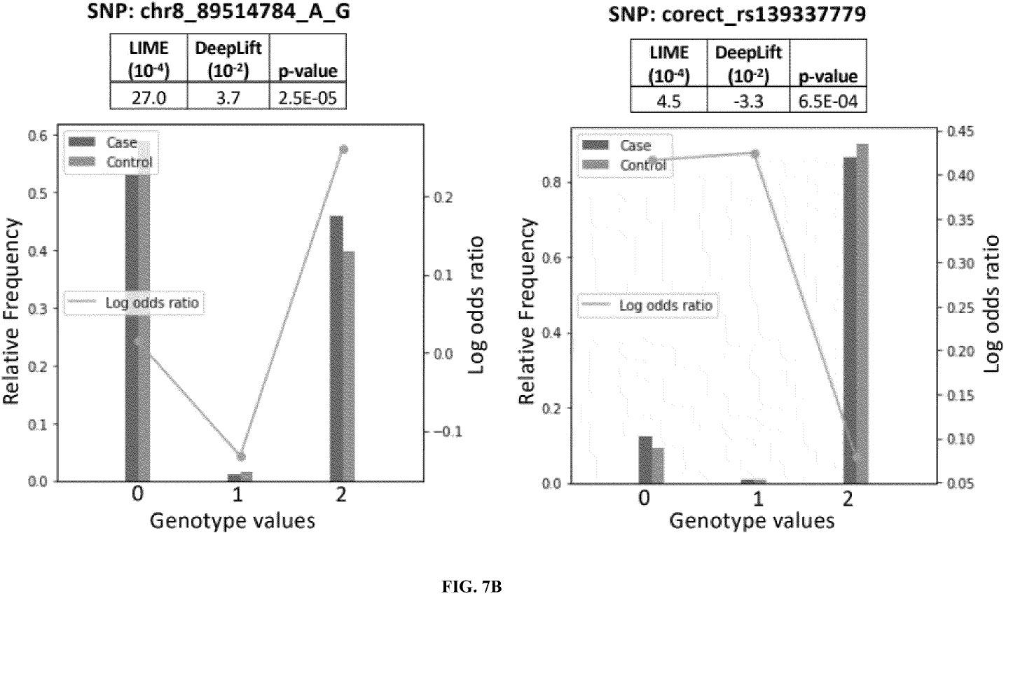

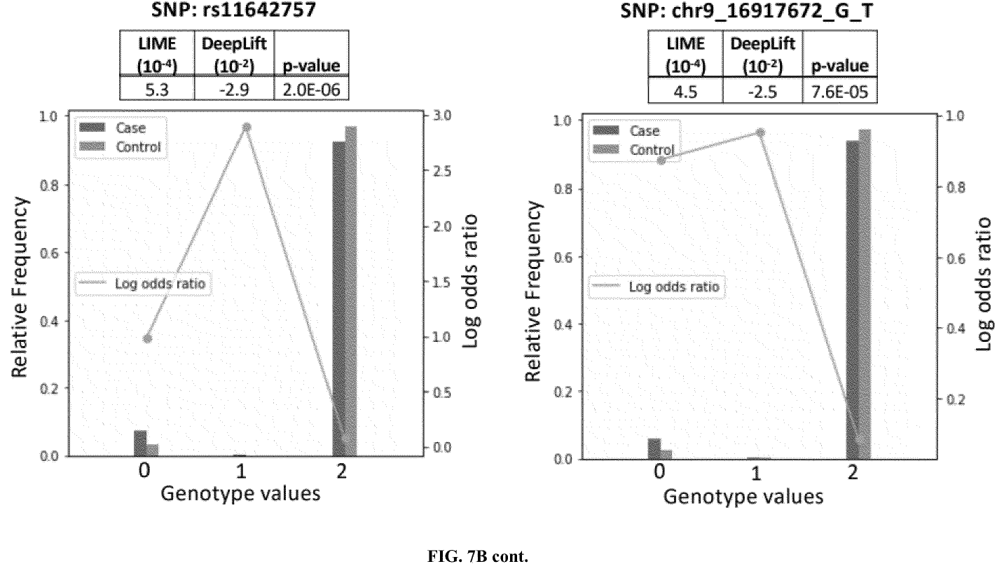

While the DNN model used 5,273 SNPs as input, only a small set of these SNPs were particularly informative for identifying the subjects with high genetic risks for breast cancer. LIME and DeepLift were used to find the top-100 salient SNPs used by the DNN model to identify the subjects with PRS higher than the 0.67 cutoff at 90% precision in the test set (FIG. 1). Twenty three SNPs were ranked by both algorithms to be among their top-100 salient SNPs (FIG. 6). The small overlap between their results can be attributed to their different interpretation approaches. LIME considered the DNN model as a black box and perturbed the input to estimate the importance of each variable; whereas, DeepLift analyzed the gradient information of the DNN model. 30% of LIME’s salient SNPs and 49% of DeepLift’s salient SNPs had p-values less than the Bonferroni significance threshold of 9.5 · 10⁻⁸. This could be attributed to the non-linear relationships between the salient SNP genotype and the disease outcome, which cannot be captured by the association analysis using logistic regression. To illustrate this, four salient SNPs with significant p-values were shown in FIG. 7A, which exhibited linear relationships between their genotype values and log odds ratios as expected. Four salient SNPs with insignificant p-values were shown in FIG. 7B, which showed clear biases towards cases or controls by one of the genotype values in a non-linear fashion.

Michailidiou et al. (2017) summarized a total of 172 SNPs associated with breast cancer. Out of these SNPs, 59 were not included on OncoArray, 63 had an association p-value less than 10⁻³ and were not included in the 5,273-SNP feature set for DNN, 34 were not ranked among the top-1000 SNPs by either DeepLIFT or LIME, and 16 were ranked among the top-1000 SNPs by DeepLIFT, LIME, or both (Table 7). This indicates that many SNPs with significant association may be missed by the interpretation of DNN models.

TABLE 7

Previously found SNPs compared with DeepLift and LIME findings

| Locus | SNP | Chromosome | Position | MAF | DeepLift Saliency Score Rank* | LIME Saliency ScoreRank* | Nearby Gene | P-value from PLINK * |
|---|---|---|---|---|---|---|---|---|
| 10p12.31 | rs7072776 | 22032942 | 10 | 0.29 | N/A | N/A | DNAJC 1 | D |
| 10p12.31 | rs11814448 | 22315843 | 10 | 0.02 | N/A | N/A | DNAJC1 | D |
| 10q25.2 | rs7904519 | 114773927 | 10 | 0.46 | N/A | N/A | TCFL2 | D |
| 10q26.12 | rs11199914 | 123093901 | 10 | 0.32 | N/A | N/A | None | D |
| 11q13.1 | rs3903072 | 65583066 | 11 | 0.47 | >1000 | >1000 | None | 1.82E-04 |
| 11q24.3 | rs1182064.6 | 129461171 | 11 | 0.4 | N/A | N/A | None | D |
| 12p13.1 | rs12422552 | 14413931 | 12 | 0.26 | N/A | N/A | None | D |
| 12q22 | rs17356907 | 96027759 | 12 | 0.3 | >1000 | 278 | NTN4 | 2.42E-08 |
| 13q13.1 | rs11571833 | 32972626 | 13 | 0.01 | >1000 | >1000 | BRCA2 | 2.90E-05 |
| 14q 13.3 | rs2236007 | 37132769 | 14 | 0.21 | >1000 | >1000 | PAX9 | 3.28E-05 |
| 14q24.1 | rs2588809 | 68660428 | 14 | 0.17 | >1000 | >1000 | RAD51B | 1.53E-04 |
| 14q32.11 | rs941764 | 91841069 | 14 | 0.35 | N/A | N/A | CCDC88C | D |
| 16q12.2 | rs17817449 | 53813367 | 16 | 0.41 | >1000 | >1000 | FTO | 1.89E-05 |
| 16q23.2 | rs13329835 | 80650805 | 16 | 0.23 | >1000 | >1000 | CDYL2 | 2.45E-07 |
| 18q11.2 | rs527616 | 24337424 | 18 | 0.38 | N/A | N/A | None | D |
| 18q11.2 | rs1436904 | 24570667 | 18 | 0.4 | N/A | N/A | CHST9 | D |
| 19p13.11 | rs4808801 | 18571141 | 19 | 0.34 | >1000 | >1000 | ELL | 3.28E-04 |
| 19q13.31 | rs3760982 | 44286513 | 19 | 0.46 | >1000 | >1000 | KCCN4, LYPD5 | 5. 16E-04 |
| 1p13.2 | rs11552449 | 114448389 | 1 | 0.17 | N/A | N/A | DCLRE1B | D |
| 1p36.22 | rs616488 | 10566215 | 1 | 0.33 | N/A | N/A | PEX14 | D |
| 22q12.1 | rs17879961 | 29121087 | 22 | 0.005 | N/A | N/A | CHEK2 | D |
| 22q12.2 | rs132390 | 29621477 | 22 | 0.04 | N/A | N/A | EM1D1 | D |
| 22q13.1 | rs6001930 | 40876234 | 22 | 0.1 | >1000 | >1000 | MKL 1 | 2.03E-07 |
| 2q14.1 | rs4849887 | 121245122 | 2 | 0.1 | N/A | N/A | | N/A |
| 2q31.1 | rs2016394 | 172972971 | 2 | 0.47 | N/A | N/A | DLX2NoneAS 1 | N/A |
| 2q31.1 | rs1550623 | 174212894 | 2 | 0.15 | >1000 | >1000 | CDCA7 | 7.07E-04 |
| 2q35 | rs16857609 | 218296508 | 2 | 0.26 | >1000 | >1000 | DIRC3 | 3.07E-06 |
| 3p.24.1 | rs12493607 | 30682939 | 3 | 0.34 | N/A | N/A | TGFBR2 | D |
| 3p26.1 | rs6762644 | 4742276 | 3 | 0.38 | >1000 | >1000 | EGOT/ITPR1 | 3.46E-08 |
| 4q24 | rs9790517 | 106084778 | 4 | 0.23 | N/A | N/A | TET2 | D |
| 4q34.1 | rs6828523 | 175846426 | 4 | 0.12 | >1000 | >1000 | ADAM29 | 7.39E-05 |
| 5q11.2 | rs10472076 | 58184061 | 5 | 0.38 | N/A | N/A | RAB3C | D |
| 5q11.2 | rs1353747 | 58337481 | 5 | 0.09 | N/A | N/A | PDE4D | D |
| 5q33.3 | rs1432679 | 158244083 | 5 | 0.43 | >1000 | >1000 | EBF1 | 7.65E-11 |
| 6p23 | rs204247 | 13722523 | 6 | 0.44 | >1000 | >1000 | RANBP9 | 4.38E-04 |
| 6p25.3 | rs11242675 | 1318878 | 6 | 0.37 | N/A | N/A | FOXQ1 | D |
| 7q35 | rs720475 | 144074929 | 7 | 0.25 | N/A | N/A | NOBOX, ARHGEF6 | D |
| 8p12 | rs9693444 | 29509616 | 8 | 0.32 | >1000 | >1000 | None | 2.45E-07 |
| 8q21.11 | rs6472903 | 76230301 | 8 | 0.17 | N/A | N/A | None | D |
| 8q21.11 | rs294-3559 | 76417937 | 8 | 0.08 | >1000 | >1000 | HNF4G | 6.82E-04 |
| 8q24.21 | rs11780156 | 129194641 | 8 | 0.17 | N/A | N/A | MYC | D |
| 9q31.2 | rs10759243 | 110306115 | 9 | 0.29 | >1000 | >1000 | None | 3.63E-06 |
| 6q25.19 | rs9485372 | 149608874 | 6 | 0.19 | N/A | N/A | TAB2 | D |
| 15q26.19 | rs2290203 | 91512067 | 15 | 0.21 | >1000 | >1000 | PRC1 | 9.74E-04 |
| 1q32.19 | rs4951011Â | 203766331 | 1 | 0.16 | N/A | N/A | ZC3H11A | D |
| 5q14.39 | rs10474352 | 90732225 | 5 | 0.16 | >1000 | >1000 | ARRDC3 | 5.71E-05 |
| 22q13.19 | chr22:39359355 | 39359355 | 22 | 0.1 | N/A | N/A | APOBEC3A,APOBEC3B | N/A |
| 14q32.12 | rs11627032 | 93104072 | 14 | 0.25 | N/A | N/A | RIN3 | D |
| 17q11.2 | rs146699004 | 29230520 | 17 | 0.27 | N/A | N/A | ATAD5 | N/A |
| 17q25.3 | rs745570 | 77781725 | 17 | 0.5 | N/A | N/A | None | D |
| 18q12.3 | rs6507583 | 42399590 | 18 | 0.07 | N/A | N/A | SETBP1 | D |
| 1q21.1 | rs12405132 | 145644984 | 1 | 0.37 | N/A | N/A | RNF115 | D |
| 1q21.2 | rs12048493 | 149927034 | 1 | 0.38 | N/A | N/A | OTUD7B | N/A |
| 1q43 | rs72755295 | 242034263 | 1 | 0.03 | N/A | N/A | EXO1 | D |
| 3p21.31 | rs6796502 | 46866866 | 3 | 0.1 | N/A | N/A | None | D |
| 5p13.3 | rs2012709 | 32567732 | 5 | 0.48 | N/A | N/A | None | D |
| 5p15.1 | rs13162653 | 16187528 | 5 | 0.45 | N/A | N/A | None | D |
| 5q14.2 | rs7707921 | 81538046 | 5 | 0.25 | >1000 | >1000 | ATG1 0 | 2.15E-04 |
| 6p22.1 | rs9257408 | 28926220 | 6 | 0.41 | N/A | N/A | None | N/A |
| 7q32.3 | rs4593472 | 130667121 | 7 | 0.3 5 | N/A | N/A | FLJ43663 | D |
| 8p11.23 | rs13365225 | 36858483 | 8 | 0.18 | >1000 | >1000 | None | 3.86E-11 |
| 8q23.3 | rs13267382 | 117209548 | 8 | 0.36 | N/A | N/A | LINC00536 | D |
| 2q35 | rs4442975 | 217920769 | 2 | 0.5 | >1000 | 408 | IGFBP5 | 5.63E-24 |
| 11p15.5 | rs3817198 | 1909006 | 11 | 0.32 | >1000 | >1000 | LSP1 | 7.99E-05 |
| 8q24.21 | rs13281615 | 128355618 | 8 | 0.41 | >1000 | >1000 | None | 4.75E-10 |
| 14q24.1 | rs999737 | 69034682 | 14 | 0.23 | >1000 | >1000 | RAD51B | 3.65E-08 |
| 1p11.2 | rs11249433 | 121280613 | 1 | 0.41 | 474 | 61 | EMBP 1 | 4.11E-17 |
| 16q 12.2 | rs11075995 | 53855291 | 16 | 0.24 | N/A | N/A | FTO | D |
| 1q32.1 | rs6678914 | 202187176 | 1 | 0.41 | N/A | N/A | LGR6 | D |
| 1q32.1 | rs4245739 | 20451884.2 | 1 | 0.26 | N/A | N/A | MDM4 | D |
| 2p24.1 | rs12710696 | 19320803 | 2 | 0.37 | N/A | N/A | None | D |
| 13q22.1 | rs6562760 | 73957681 | 13 | 0.24 | N/A | N/A | None | D |
| 2p23.2 | rs4577244 | 29120733 | 2 | 0.23 | N/A | N/A | WDR43 | D |
| 2q33.1 | rs1830298 | 202181247 | 2 | 0.28 | N/A | N/A | CASP8/ALS2CR12 | N/A |
| 2q35 | rs34005590 | 217963060 | 2 | 0.05 | N/A | N/A | IGFBP5 | N/A |
| 3p24.1 | rs4973768 | 27416013 | 3 | 0.47 | >1000 | >1000 | SLC4A7 | 1.61E-06 |
| 3p14.1 | rs1053338 | 63967900 | 3 | 0.14 | N/A | N/A | ATNX7 | D |
| 7q21.2 | rs6964587 | 91630620 | 7 | 0.39 | N/A | N/A | AKAP9 | D |
| 5p15.33 | rs10069690 | 1279790 | 5 | 0.26 | >1000 | >1000 | TERT | 9.50E-04 |
| 5p15.33 | rs3215401 | 1296255 | 5 | 0.3 1 | >1000 | 68 | TERT | 3.71E-07 |
| 5p12 | rs10941679 | 44706498 | 5 | 0.25 | 392 | >1000 | FGF10, MRPS30 | 8.06E-17 |
| 5q11.2 | rs62355902 | 56053723 | 5 | 0.16 | N/A | N/A | MAP3K1 | D |
| 6p24.3 | rs9348512 | 10456706 | 6 | 0.33 | N/A | N/A | TFAP2A | N/A |
| 20q11.22 | rs2284378Â | 32588095 | 20 | 0.32 | N/A | N/A | RALY | D |
| 6q14.1 | rs17529111 | 82128386 | 6 | 0.22 | N/A | N/A | None | N/A |
| 6q25 | rs3757322 | 151942194 | 6 | 0.32 | >1000 | >1000 | ESR1 | 8.02E-09 |
| 6q25 | rs9397437 | 151952332 | 6 | 0.07 | 746 | >1000 | ESR1 | 3.37E-07 |
| 6q25 | rs2747652 | 152437016 | 6 | 0.48 | N/A | N/A | ESR1 | D |
| 7q34 | rs11977670 | 139942304 | 7 | 0.43 | 950 | 608 | None | 1.51E-04 |
| 10p15.1 | rs2380205 | 5886734 | 10 | 0.44 | N/A | N/A | ANKRD16 | D |
| 10q22.3 | rs704010 | 80841148 | 10 | 0.3 8 | >1000 | >1000 | ZMZ1 | 9.95E-07 |
| 9p21.3 | rs1011970 | 22062134 | 9 | 0.16 | 982 | >1000 | CDKN2A,CDKN2B | 3.04E-04 |
| 9q31.2 | rs676256A | 110895353 | 9 | 0.38 | >1000 | >1000 | None | 3.85E-08 |
| 9q31.2 | rs10816625 | 110837073 | 9 | 0.06 | N/A | N/A | None | D |
| 9q31.2 | rs13294895 | 110837176 | 9 | 0.18 | N/A | N/A | None | D |
| 10q21.2 | rs10995201Â | 64299890 | 10 | 0.16 | N/A | N/A | ZNF365 | N/A |
| 10q26.13 | rs35054928 | 123340431 | 10 | 0.4 | N/A | N/A | FGFR2 | N/A |
| 10q26.13 | rs45631563 | 123349324 | 10 | 0.05 | N/A | N/A | FGFR2 | N/A |
| 10q26.13 | rs2981578 | 123340311 | 10 | 0.47 | >1000 | 748 | FGFR2 | 1.39E-35 |
| 11q13.3 | rs554219 | 69331642 | 11 | 0.13 | 418 | >1000 | CCND1 | 2.71E-17 |
| 11q13.3 | rs75915166 | 69379161 | 11 | 0.06 | >1000 | 841 | CCND1 | 4.86E-14 |
| 12p11.22 | rs7297051 | 28174817 | 12 | 0.24 | >1000 | >1000 | None | 7.40E-09 |
| 12q24.21 | rs1292011 | 115836522 | 12 | 0.42 | N/A | N/A | TBX3 | N/A |
| 21q21.1 | rs2823093 | 16520832 | 21 | 0.27 | >1000 | >1000 | NRIP1 | 5.29E-04 |
| 16q12.1 | rs4784227 | 52599188 | 16 | 0.24 | >1000 | 396 | TOX3 | 1.00E-34 |
| 17q22 | rs2787486 | 53209774 | 17 | 0.3 | N/A | N/A | None | N/A |
| 19p13.11 | rs67397200 | 17401404 | 19 | 0.3 | N/A | N/A | None | D |
| 10p14 | rs67958007 | 9088113 | 10 | 0.12 | N/A | N/A | None | N/A |
| 10q23.33 | rs140936696 | 95292187 | 10 | 0.18 | N/A | N/A | None | N/A |
| 11p15 | rs6597981 | 803017 | 11 | 0.48 | N/A | N/A | PIDD1 | N/A |
| 12q21.31 | rs202049448 | 85009437 | 12 | 0.34 | N/A | N/A | None | N/A |
| 12q24.31 | rs206966 | 120832146 | 12 | 0.16 | N/A | N/A | None | D |
| 14q32.33 | rs10623258 | 105212261 | 14 | 0.45 | N/A | N/A | ADSSL1 | N/A |
| 16q12.2 | rs28539243 | 54682064 | 16 | 0.49 | N/A | N/A | None | D |
| 16q13 | rs2432539 | 56420987 | 16 | 0.4 | N/A | N/A | AMFR | N/A |
| 16q24.2 | rs4496150 | 87085237 | 16 | 0.25 | N/A | N/A | None | N/A |
| 17q21.2 | rs72826962 | 40836389 | 17 | 0.01 | N/A | N/A | CNTNAP1 | D |
| 17q21.31 | rs2532263 | 44252468 | 17 | 0.19 | N/A | N/A | KANSL1 | N/A |
| 18q12.1 | rs117618124 | 29977689 | 18 | 0.05 | N/A | N/A | GAREM1 | N/A |
| 19p13.11 | rs2965183 | 19545696 | 19 | 0.35 | N/A | N/A | GATAD2A, MIR640 | D |
| 19p13.12 | rs2594714 | 13954571 | 19 | 0.23 | N/A | N/A | None | N/A |
| 19p13.13 | rs78269692 | 13158277 | 19 | 0.05 | N/A | N/A | NFIX1 | D |
| 19q13.22 | rs71338792 | 46183031 | 19 | 0.23 | N/A | N/A | GIPR | N/A |
| 1p12 | rs7529522 | 118230221 | 1 | 0.23 | N/A | N/A | None | N/A |
| 1p22.3 | rs17426269 | 88156923 | 1 | 0.15 | N/A | N/A | None | D |
| 1p32.3 | rs140850326 | 50846032 | 1 | 0.49 | N/A | N/A | None | N/A |
| 1p34.1 | rs1707302 | 46600917 | 1 | 0.34 | N/A | N/A | PIK3R3, LOC101929626 | D |
| 1p34.2 | rs4233486 | 41380440 | 1 | 0.36 | N/A | N/A | None | N/A |
| 1p34.2 | rs79724016 | 42137311 | 1 | 0.03 | N/A | N/A | HIVEP3 | N/A |
| 1p36.13 | rs2992756 | 18807339 | 1 | 0.49 | N/A | N/A | KLHDC7A | N/A |
| 1q22 | rs4971059 | 155148781 | 1 | 0.35 | >1000 | >1000 | TRIM46 | 4.66E-04 |
| 1q32.1 | rs35383942 | 201437832 | 1 | 0.06 | >1000 | >1000 | PHLDA3 | 9.80E-05 |
| 1q41 | rs11117758 | 217220574 | 1 | 0.21 | N/A | N/A | ESRRG | N/A |
| 20p12.3 | rs16991615 | 5948227 | 20 | 0.06 | 881 | >1000 | MCM8 | 1.43 E-04 |
| 20q13.13 | rs6122906 | 48945911 | 20 | 0.18 | N/A | N/A | None | D |
| 22q13.1 | rs738321 | 38568833 | 22 | 0.38 | N/A | N/A | PLA2G6 | N/A |
| 22q13.2 | rs73161324 | 42038786 | 22 | 0.06 | N/A | N/A | XRCC6 | D |
| 22q13.31 | rs28512361 | 46283297 | 22 | 0.11 | N/A | N/A | None | N/A |
| 2p23.3 | rs6725517 | 25129473 | 2 | 0.41 | N/A | N/A | ADCY3 | D |
| 2p25.1 | rs113577745 | 10135681 | 2 | 0.1 | N/A | N/A | GRHL1 | N/A |
| 2q13 | rs71801447 | 111925731 | 2 | 0.06 | N/A | N/A | BCL2L11 | N/A |
| 2q36.3 | rs12479355 | 227226952 | 2 | 0.21 | N/A | N/A | None | N/A |
| 3p12.1 | rs13066793 | 87037543 | 3 | 0.09 | N/A | N/A | VGLL3 | D |
| 3p12.1 | rs9833888 | 99723580 | 3 | 0.22 | N/A | N/A | CMSS1, FILIP1L | D |
| 3p13 | rs6805189 | 71532113 | 3 | 0.48 | N/A | N/A | FOXP1 | N/A |
| 3q23 | rs34207738 | 141112859 | 3 | 0.41 | N/A | N/A | ZBTB38 | N/A |
| 3q26.31 | rs58058861 | 172285237 | 3 | 0.21 | N/A | N/A | None | N/A |
| 4p14 | rs6815814 | 38816338 | 4 | 0.26 | N/A | N/A | None | N/A |
| 4q21.23 | 4:84370124 | 84370124 | 4 | 0.47 | N/A | N/A | HELQ | N/A |
| 4q22.1 | rs10022462 | 89243818 | 4 | 0.44 | 964 | >1000 | LOC105369192 | 1.88E-04 |
| 4q28.1 | rs77528541 | 126843504 | 4 | 0.13 | N/A | N/A | None | N/A |
| 5p15.33 | rs116095464 | 345109 | 5 | 0.05 | N/A | N/A | AHRR | N/A |
| 5q11.1 | rs72749841 | 49641645 | 5 | 0.16 | N/A | N/A | None | N/A. |
| 5q11.1 | rs35951924 | 50195093 | 5 | 0.32 | N/A | N/A | None | N/A |
| 5q22.1 | rs6882649 | 111217786 | 5 | 0.34 | N/A | N/A | NREP | N/A |
| 5q31.1 | rs6596100 | 132407058 | 5 | 0.25 | N/A | N/A | HSPA4 | N/A |
| 5q35.1 | rs4562056 | 169591487 | 5 | 0.33 | N/A | N/A | None | N/A |
| 6p22.2 | rs71557345 | 26680698 | 6 | 0.07 | N/A | N/A | None | N/A |
| 6p22.3 | rs3819405 | 16399557 | 6 | 0.33 | N/A | N/A | ATXN1 | D |
| 6p22.3 | rs2223621 | 20621238 | 6 | 0.38 | N/A | N/A | CDKAL1 | N/A |
| 6q14.1 | rs12207986 | 81094287 | 6 | 0.47 | N/A | N/A | None | N/A |
| 6q23.1 | rs6569648 | 130349119 | 6 | 0.24 | >1000 | 941 | L3MBTL3 | 1.77E-04 |
| 7p15.1 | rs17156577 | 28356889 | 7 | 0.11 | N/A | N/A | CREB5 | D |
| 7p15.3 | rs7971 | 21940960 | 7 | 0.35 | N/A | N/A | DNAH11, CDCA7L | N/A |
| 7q21.3 | rs17268829 | 94113799 | 7 | 0.28 | N/A | N/A | None | N/A |
| 7q22.1 | rs71559437 | 101552440 | 7 | 0.12 | N/A | N/A | CUX1 | N/A. |
| 8q22.3 | rs514192 | 102478959 | 8 | 0.32 | 258 | >1000 | None | 1.71E-04 |
| 8q23.1 | rs12546444 | 106358620 | 8 | 0.1 | N/A | N/A | ZFPM3 | N/A |
| 8q24.13 | rs58847541 | 124610166 | 8 | 0.15 | N/A | N/A | None | N/A |
| 9q33.1 | rs1895062 | 119313486 | 9 | 0.41 | N/A | N/A | ASTN2 | N/A |
| 9q33.3 | rs10760444 | 129396434 | 9 | 0.43 | N/A | N/A | LMX1B | D |
| 9q34.2 | rs8176636 | 136151579 | 9 | 0.2 | N/A | N/A | ABO | N/A |

*N/A: Not present in the OncoArray; D: Discarded by the association analysis

The 23 salient SNPs identified by both DeepLift and LIME in their top-100 list are shown in Table 2. Eight of the 23 SNPs had p-values higher than the Bonferroni level of significance and were missed by the association analysis using Plink. The potential oncogenesis mechanisms for some of the 8 SNPs have been investigated in previous studies. The SNP, rs139337779 at 12q24.22, is located within the gene, Nitric oxide synthase 1 (NOS1). Li et al. (Li et al., 2019) showed that the overexpression of NOS1 can up-regulate the expression of ATP-binding cassette, subfamily G, member 2 (ABCG2), which is a breast cancer resistant protein (Mao & Unadkat, 2015), and NOS1-indeuced chemo-resistance was partly mediated by the up-regulation of ABCG2 expression. Lee et al. (2009) reported that NOS1 is associated with the breast cancer risk in a Korean cohort. The SNP, chr13_113796587_A_G at 13q34, is located in the F10 gene, which is the coagulation factor X. Tinholt et al. (2014) showed that the increased coagulation activity and genetic polymorphisms in the F10 gene are associated with breast cancer. The BNC2 gene containing the SNP, chr9_16917672_G_T at 9p22.2, is a putative tumor suppressor gene in high-grade serious ovarian carcinoma (Casaratto et al., 2016). The SNP, chr2_171708059_C_T at 2q31.1, is within the GAD1 gene and the expression level of GAD1 is a significant prognostic factor in lung adenocarcinoma (Tsuboi et al., 2019). Thus, the interpretation of DNN models may identify novel SNPs with non-linear association with the breast cancer.

### Example 5 - LINA: A Linearizing Neural Network Architecture for Accurate First-order and Second-Order Interpretations

While neural networks can provide high predictive performance, it has been a challenge to identify the salient features and important feature interactions used for their predictions. This represented a key hurdle for deploying neural networks in many biomedical applications that require interpretability, including predictive genomics. In this paper, linearizing neural network architecture (LINA) was developed here to provide both the first-order and the second-order interpretations on both the instance-wise and the model-wise levels. LINA combines the representational capacity of a deep inner attention neural network with a linearized intermediate representation for model interpretation. In comparison with DeepLIFT, LIME, Grad*Input and L2X, the first-order interpretation of LINA had better Spearman correlation with the ground-truth importance rankings of features in synthetic datasets. In comparison with NID and GEH, the second-order interpretation results from LINA achieved better precision for identification of the ground-truth feature interactions in synthetic datasets. These algorithms were further benchmarked using predictive genomics as a real-world application. LINA identified larger numbers of important single nucleotide polymorphisms (SNPs) and salient SNP interactions than the other algorithms at given false discovery rates. The results showed accurate and versatile model interpretation using LINA.

An interpretable machine learning algorithm should have a high representational capacity to provide strong predictive performance, and its learned representations should be amenable to model interpretation and understandable to humans. The two desiderata are generally difficult to balance. Linear models and decision trees generate simple representations for model interpretation, but have low representational capacities for only simple prediction tasks. Neural networks and support vector machines have high representational capacities to handle complex prediction tasks, but their learned representations are often considered to be “black-boxes” for model interpretation (Bermeitinger et al., 2019).

Predictive genomics is an exemplar application that requires both a strong predictive performance and high interpretability. In this application, the genotype information for a large number of SNPs in a subject’s genome is used to predict the phenotype of this subject. While neural networks have been shown to provide better predictive performance than statistical models (Badré et al., 2020; Fergus et al., 2018), statistical models are still the dominant methods for predictive genomics, because geneticists and genetic counselors can understand which SNPs are used and how they are used as the basis for certain phenotype predictions. Neural network models have also been used in many other important bioinformatics applications (Ho Thanh Lam et al., 2020; Do & Le, 2020; Baltres et al., 2020) that can benefit from model interpretation.

To make neural networks more useful for predictive genomics and other applications, in certain non-limiting embodiments, the present disclosure is directed to a new neural network architecture, referred to as linearizing neural network architecture (LINA), to provide both first-order and second-order interpretations and both instance-wise and model-wise interpretations.

Model interpretation reveals the input-to-output relationships that a machine learning model has learned from the training data to make predictions (Molnar, 2019). The first-order model interpretation aims to identify individual features that are important for a model to make predictions. For predictive genomics, this can reveal which individual SNPs are important for phenotype prediction. The second-order model interpretation aims to identify important interactions among features that have a large impact on model prediction. The second-order interpretation may reveal the XOR interaction between the two features that jointly determine the output. For predictive genomics, this may uncover epistatic interactions between pairs of SNPs (Cordell, 2002; Phillips, 2008).

A general strategy for the first-order interpretation of neural networks, first introduced by Saliency (Simonyan et al., 2014), is based on the gradient of the output with respect to (w.r.t.) the input feature vector. A feature with a larger partial derivative of the output is considered more important. The gradient of a neural network model w.r.t. the input feature vector of a specific instance can be computed using backpropagation, which generates an instance-wise first-order interpretation. The Grad*Input algorithm (Shrikumar et al., 2017) multiplies the obtained gradient element-wise with the input feature vector to generate better scaled importance scores. As an alternative to using the gradient information, the Deep Learning Important FeaTures (DeepLIFT) algorithm explains the predictions of a neural network by backpropagating the activations of the neurons to the input features (Shrikumar et al., 2017). The feature importance scores are calculated by comparing the activations of the neurons with their references, which allows the importance information to pass through a zero gradient during backpropagation. The Class Model Visualization (CMV) algorithm (Simonyan et al., 2014) computes the visual importance of pixels in convolution neural network (CNN). It performs backpropagation on an initially dark image to find the pixels that maximize the classification score of a given class.

While the algorithms described above were developed specifically for neural networks, model-agnostic interpretation algorithms can be used for all types of machine learning models. Local Interpretable Model-agnostic Explanations (LIME) (Ribeiro et al., 2016) fits a linear model to synthetic instances that have randomly perturbed features in the vicinity of an instance. The obtained linear model is analyzed as a local surrogate of the original model to identify the important features for the prediction on this instance. Because this approach does not rely on gradient computation, LIME can be applied to any machine learning model, including non-differentiable models. The studies in Examples 1-4 combined LIME and DeepLIFT to interpret a feedforward neural network model for predictive genomics. Kernel SHapley Additive exPlanations (SHAP) (Lundberg & Lee, 2022) uses a sampling method to find the Shapley value for each feature of a given input. The Multi-Objective Counterfactuals (MOC) method (Dandl et al., 2020) searches for the counterfactual explanations for an instance by solving a multi-objective optimization problem. The importance scores calculated by the L2X algorithm (Chen et al., 2021) are based on the mutual information between the features and the output from a machine learning model. L2X is efficient because it approximates the mutual information using a variational approach.

The second-order interpretation is more challenging than the first-order interpretation because d features would have (d² - d)/2 possible interactions to be evaluated. Computing the Hessian matrix of a model for the second-order interpretation is conceptually equivalent to, but much more computationally expensive than, computing the gradient for the first-order interpretation. Group Expected Hessian (GEH) (Cui et al., 2020) computes the Hessian of a Bayesian neural network for many regions in the input feature space and aggregates them to estimate an interaction score for every pair of features. The additive grooves algorithm (Sorokina et al., 2007) estimates the feature interaction scores by comparing the predictive performance of the decision tree containing all features with that of the decision trees with pairs of features removed. Neural Interaction Detection (NID) (Tsang et al., 2018) avoids the high computational cost of evaluating every feature pair by directly analyzing the weights in a feedforward neural network. If some features are strongly connected to a neuron in the first hidden layer and the paths from that neuron to the output have high aggregated weights, then NID considers these features to have strong interactions.

Model interpretations can be further classified as instance-wise interpretations or model-wise interpretations. Instance-wise interpretation algorithms, including Saliency (Simonyan et al., 2014), LIME (Ribeiro et al., 2016) and L2X (Chen et al., 2018), provide an explanation for a model’s prediction for a specific instance. For example, an instance-wise interpretation of a neural network model for predictive genomics may highlight the important SNPs in a specific subject which are the basis for the phenotype prediction of this subject. This is useful for intuitively assessing how well grounded the prediction of a model is for a specific subject. Model-wise interpretation provides insights into how a model makes predictions in general. CMV (Simonyan et al., 2014) was developed to interpret CNN models. Instance-wise interpretation methods can also be used to explain a model by averaging the explanations of all the instances in a test set. A model-wise interpretation of a predictive genomics model can reveal the important SNPs for a phenotype prediction in a large cohort of subjects. Model-wise interpretations shed light on the internal mechanisms of a machine learning model.

Disclosed herein is a LINA architecture and first-order and second-order interpretation algorithms for LINA. The interpretation performance of the new methods has been benchmarked using synthetic datasets and a predictive genomics application in comparison with state-of-the-art (SOTA) interpretation methods. The interpretations from LINA were more versatile and more accurate than those from the SOTA methods.

### Methods

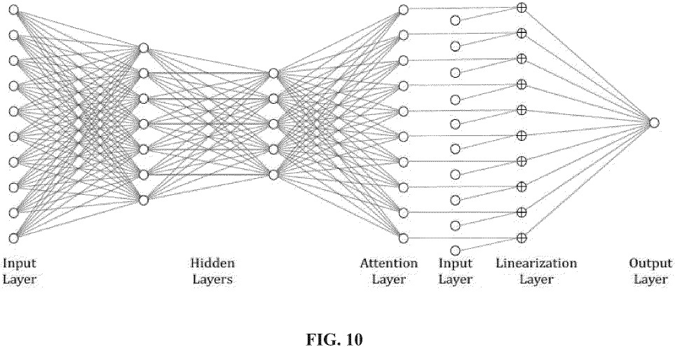

(A) LINA Architecture. The key feature of the LINA architecture (FIG. 10) is the linearization layer, which computes the output as an element-wise multiplication product of the input features, attention weights, and coefficients:

$$y = S \left[{K}^{T} \left(A ∘ X\right) + b\right] = S \left(\sum {}_{i = 1}^{d} {k}_{i} {a}_{i} {x}_{i} + b\right)$$

where y is the output, X is the input feature vector, S() is the activation function of the output layer, ◦ represents the element-wise multiplication operation, K and b are respectively the coefficient vector and bias that are constant for all instances, and A is the attention vector that adaptively scales the feature vector of an instance. X, A and K are three vectors of dimension d, which is the number of input features. The computation by the linearization layer and the output layer is also expressed in a scalar format in Equation (1). This formulation allows the LINA model to learn a linear function of the input feature vector, coefficient vector, and attention vector.

The attention vector is computed from the input feature vector using a multi-layer neural network, referred to as the inner attention neural network in LINA. The inner attention neural network must be sufficiently deep for a prediction task owing to the designed low representational capacity of the remaining linearization layer in a LINA model. In the inner attention neural network, all hidden layers use a non-linear activation function, such as ReLU, but the attention layer uses a linear activation function to avoid any restriction in the range of the attention weights. This is different from the typical attention mechanism in existing attentional architectures which generally use the softmax activation function.

(B) The Loss Function. The loss function for LINA is composed of the training error loss, regularization penalty on the coefficient vector, and regularization penalty on the attention vector:

$$l o s s = E \left(Y , {Y}_{t r u e}\right) + β {\left‖K\right‖}_{2} + γ {\left‖A - 1\right‖}_{1}$$

where E is a differentiable convex training error function, ||K||₂ is the L2 norm of the coefficient vector, ||A - 1||₁ is the L1 norm of the attention vector minus 1, and β and y are the regularization parameters. The coefficient regularization sets 0 to be the expected value of the prior distribution for K, which reflects the expectation of un-informative features. The attention regularization sets 1 to be the expected value of the prior distribution for A, which reflects the expectation of a neutral attention weight that does not scale the input feature. The values of β and y and the choices of L2, L1, and L0 regularization for the coefficient and attention vectors are all hyperparameters that can be optimized for predictive performance on the validation set.

(C) First-order Interpretation. LINA derives the instance-wise first-order interpretation from the gradient of the output, y, w.r.t the input feature vector, X. The output gradient can be decomposed as follows:

$$\frac{\partial y}{\partial {x}_{i}} = {k}_{i} {a}_{i} + \sum {}_{j = 1}^{d} {k}_{j} \frac{\partial {a}_{j}}{\partial {x}_{i}} {x}_{j}$$

Proof:
Let us derive
$$\frac{\partial y}{\partial {x}_{i}}$$
for a regression task:
$$\frac{\partial y}{\partial {x}_{i}} = \frac{\partial {k}_{i} {a}_{i} {x}_{i}}{\partial {x}_{i}} + {{\sum }_{j = 1}^{d} \frac{\partial {k}_{j} {a}_{j} {x}_{j}}{\partial {x}_{i s}}}_{j \neq i} + \frac{\partial b}{\partial {x}_{i}}$$
$$\begin{matrix}= {k}_{i} \frac{\partial \left({a}_{i} {x}_{i}\right)}{\partial {x}_{i}} + {{\sum }_{j = 1}^{d} {k}_{j}}_{j \neq i \text{}} \frac{\partial \left({a}_{j} {x}_{j}\right)}{\partial {x}_{i}} \\ = {k}_{i} \left(\frac{\partial {a}_{i}}{\partial {x}_{i}} {x}_{i} + {a}_{i}\right) + {{\sum }_{j = 1}^{d} {k}_{j}}_{j \neq i \text{}} \frac{\partial {a}_{j}}{\partial {x}_{i}} {x}_{j} \\ = {k}_{i} {a}_{i} + {\sum }_{j = 1}^{d} {k}_{j} \frac{\partial {a}_{j}}{\partial {x}_{i}} {x}_{j}\end{matrix}$$
End-of-proof.

The decomposition of the output gradient in LINA shows that the contribution of a feature in an attentional architecture comprises (i) a direct contribution to the output weighted by its attention weight and (ii) an indirect contribution to the output during attention computation. This indicates that using attention weights directly as a measure of feature importance omits the indirect contribution of a feature in the attention mechanism.

For the instance-wise first-order interpretation, the inventors defined

$$F {Q}_{i} = \frac{\partial y}{\partial {x}_{i}}$$

as the full importance score for feature i,

$$D {Q}_{i} = {k}_{i} {a}_{i}$$

as the direct importance score for feature i,

$$I {Q}_{i} = \sum {}_{j = 1}^{d} {k}_{j} \frac{\partial {a}_{j}}{\partial {x}_{i}}$$

and as the indirect importance score for feature i.

For the model-wise first-order interpretation, the inventors defined the model-wise full importance score (FPᵢ), direct importance score (DPᵢ), and indirect importance score (IPᵢ) for feature i as the averages of the absolute values of the corresponding instance-wise importance scores of this feature across all instances in the test set:

$${\text{FP}}_{\text{i}} = \overline{\left|{\text{FQ}}_{\text{i}}\right|}$$

$${\text{DP}}_{\text{i}} = \overline{\left|{\text{DQ}}_{\text{i}}\right|}$$

$${\text{IP}}_{\text{i}} = \overline{\left|{\text{IQ}}_{\text{i}}\right|}$$

Because absolute values are used, the model-wise FPᵢ of feature i is no longer a sum of its IPᵢ and DPᵢ.

(D) Second-order Interpretation. It is computationally expensive and unscalable to compute the Hessian matrix for a large LINA model. Here, the Hessian matrix of the output w.r.t. the input feature vector is approximated using the Jacobian matrix of the attention vector w.r.t. the input feature vector in a LINA model, which is computationally feasible to calculate. An approximation is derived as follows.

$$\frac{{\partial }^{2} y}{\partial {x}_{i} \partial {x}_{j}} = {K}^{T} \frac{\partial }{\partial {x}_{i}} \left[\begin{matrix}{x}_{1} \frac{\partial {a}_{1}}{\partial {x}_{j}} \\ ⋮ \\ {x}_{j - 1} \frac{\partial {a}_{j - 1}}{\partial {x}_{j}} \\ {x}_{j} \frac{\partial {a}_{j}}{\partial {x}_{j}} + {α}_{j} \\ {x}_{j + 1} \frac{\partial {a}_{j + 1}}{\partial {x}_{j}} \\ ⋮ \\ {x}_{n} \frac{\partial {a}_{n}}{\partial {x}_{j}}\end{matrix}\right] = {K}^{T} \left[\begin{matrix}{x}_{1} \frac{{\partial }^{2} {a}_{1}}{\partial {x}_{i} \partial {x}_{j}} \\ ⋮ \\ {x}_{i - 1} \frac{{\partial }^{2} {a}_{j - 1}}{\partial {x}_{i} \partial {x}_{j}} \\ {x}_{i} \frac{{\partial }^{2} {a}_{i + 1}}{\partial {x}_{i} \partial {x}_{j}} + \frac{\partial {a}_{j}}{\partial {x}_{j}} \\ {x}_{i + 1} \frac{{\partial }^{2} {a}_{i + 1}}{\partial {x}_{i} \partial {x}_{j}} \\ ⋮ \\ {x}_{j - 1} \frac{{\partial }^{2} {a}_{j - 1}}{\partial {x}_{i} \partial {x}_{j}} \\ {x}_{j} \frac{{\partial }^{2} {a}_{j}}{\partial {x}_{i} \partial {x}_{j}} + \frac{\partial {a}_{j}}{\partial {x}_{i}} \\ {x}_{j - 1} \frac{{\partial }^{2} {a}_{j + 1}}{\partial {x}_{i} \partial {x}_{j}} \\ ⋮ \\ {x}_{n} \frac{{\partial }^{2} {a}_{n}}{\partial {x}_{i} \partial {x}_{j}}\end{matrix}\right]$$

By omitting the second-order derivatives of the attention weights, Equation (10) can be simplified as

$$\frac{{\partial }^{2} y}{\partial {x}_{i} \partial {x}_{j}} \approx {k}_{j} \frac{\partial {a}_{j}}{\partial {x}_{i}} + {k}_{i} \frac{\partial {a}_{i}}{\partial {x}_{j}}$$

Equation (11) shows an approximation of the Hessian of the output using the Jacobian of the attention vector. The k-weighted sum of the omitted second-order derivatives of the attention weights constitutes the approximation error. The performance of the second-order interpretation based on this approximation is benchmarked using synthetic and real-world datasets.

For instance-wise second-order interpretation, the inventors define a directed importance score of feature r to feature c:

$${\text{SQ}}_{\text{r}}^{\text{c}} = {k}_{c} \frac{\partial {a}_{c}}{\partial {x}_{r}}$$

This measures the importance of feature r in the calculation of the attention weight of feature c. In other words, this second-order importance score measures the importance of feature r to the direct importance score of feature c for the output.

For the model-wise second-order interpretation, the inventors defined an undirected importance score between feature r and feature c based on their average instance-wise second-order importance score in the test set:

$${\text{SP}}_{\text{c,r}} = \begin{matrix}\left|{\text{SQ}}_{\text{r}}^{c} + {\text{SQ}}_{c}^{r}\right|\end{matrix}$$

(E) Recap of the LINA Importance Scores. The notations and definitions of all the importance scores for a LINA model are recapitulated below in Table 8. FQ and SQ are selected as the first-order and second-order importance score, respectively, for instance-wise interpretation. FP and SP are used as the first-order and second-order importance scores, respectively, for model-wise interpretation.

TABLE 8

Notations and definitions for LINA model

| | | | |
|---|---|---|---|
| Order | Target | Acronym | Definition |
| First-order | Instance-wise | FQ | FQᵢ = DQᵢ + IQᵢ |
| | | DQ | DQᵢ = kᵢaᵢ |
| | | IQ | ${\text{IQ}}_{\text{i}} = {\sum }_{c = 1}^{d} {\text{SQ}}_{i}^{c} {x}_{c}$ |
| | Model-wise | FP | FPᵢ = \|̅F̅Q̅ᵢ̅\|̅ |
| | | DP | DPᵢ = \|̅D̅Q̅ᵢ̅\|̅ |
| | | IP | IPᵢ = \|̅I̅Q̅ᵢ̅\|̅ |
| Second-order | Instance-wise | SQ | ${\text{SQ}}_{\text{r}}^{\text{c}} = {k}_{c} \frac{\partial {a}_{c}}{\partial {x}_{r}}$ |
| | Model-wise | SP | SP_{c,r} = $\overline{\left\|S {Q}_{r}^{c} + S {Q}_{c}^{r}\right\|}$ |

### Data And Experimental Setup

(A) California housing dataset. The California housing dataset (Kelley & Barry, 1997) was used to formulate a simple regression task, which is the prediction of the median sale price of houses in a district based on eight input features (Table 5). The dataset contained 20,640 instances (districts) for model training and testing.

(B) First-order benchmarking datasets. Five synthetic datasets, each containing 20,000 instances, were created using the sigmoid functions to simulate binary classification tasks. These functions were created following the examples in (Chen et al., 2018) for the first-order interpretation benchmarking. All five datasets included ten input features. The values of the input features were independently sampled from a standard Gaussian distribution: xᵢ~N(0, 1), i ∈ {1, 2, ..., 10}. The target value was set to 0, if the sigmoid function output is (0, 0.5). The target value was set to 1, if the sigmoid function output is [0.5, 1). The inventors used the following five sigmoid functions of different subsets of the input features:

(F1):

$$S i g \left(4 ∗ {X}_{1}^{2} - 3 ∗ {X}_{2}^{2} - 2 ∗ {X}_{3}^{2} + {X}_{4}^{2}\right)$$

. This function contains four important features with independent squared relationships with the target. The ground-truth rankings of the features by first-order importance are X₁, X₂, X₃, and X₄. The remaining six uninformative features are tied in the last rank.

(F2):

$$S i g \left(- 10 ∗ sin \left({X}_{1}\right) + 2 ∗ a b s \left({X}_{2}\right) + {X}_{3} - exp \left(- {X}_{4}\right)\right)$$

. This function contains four important features with various non-linear additive relationships with the target. The ground-truth ranking of the features is X₁, X₄, X₂, and X₃. The remaining six uninformative features are tied in the last rank.

(F3):

$$S i g \left(4 ∗ {X}_{1} ∗ {X}_{2} ∗ {X}_{3} + {X}_{4} ∗ {X}_{5} ∗ {X}_{6}\right)$$

. This function contains six important features with multiplicative interactions among one another. The ground-truth ranking of the features is X₁, X₂ and X₃ tied in the first rank, X₄, X₅ and X₆ tied in the second rank, and the remaining uninformative features tied in the third rank.

(F4):

$$S i g \left(- 10 ∗ sin \left({X}_{1} ∗ {X}_{2} ∗ {X}_{3}\right) + a b s \left({X}_{4} ∗ {X}_{5} ∗ {X}_{6}\right)\right)$$

. This function contains six important features with multiplicative interactions among one another and non-linear relationships with the target. The ground-truth ranking of the features is X₁, X₂ and X₃ tied in the first rank, X₄, X₅ and X₆ tied in the second rank, and the other four uninformative features tied in the third rank.

(F5):

$$S i g \left(- 20 ∗ sin \left({X}_{1} ∗ {X}_{2}\right) + 2 ∗ a b s \left({X}_{3}\right) + {X}_{4} ∗ {X}_{5} - 4 exp \left(- {X}_{6}\right)\right)$$

. This function contains six important features with a variety of non-linear relationships with the target. The ground-truth ranking of the features is X₁ and X₂ tied in the first rank, X₆ in the second, X₃ in the third, X₄ and X₅ tied in the fourth, and the remaining uninformative features tied in the fifth.

(C) Second-order benchmarking dataset. Ten regression synthetic datasets, referred to as F6-A, F7-A, F8-A, F9-A, and F10-A (-A datasets) and F6-B, F7-B, F8-B, F9-B, and F10-B (-B datasets) were created. The -A datasets followed the examples in Tsang et al. (2018) for the second-order interpretation benchmarking. The -B datasets used the same functions below to compute the target as the -A datasets, but included more uninformative features to benchmark the interpretation performance on high-dimensional data. Each -A dataset contained 5,000 instances. Each -B dataset contained 10,000 instances. The five -A datasets included 13 input features. The five -B datasets included 100 input features, some of which were used to compute the target. In F7-A/B, F8-A/B, F9-A/B, and F10-A/B, the values of the input features of an instance were independently sampled from a standard uniform distribution: Xᵢ~U(-1,1), i ∈ {1, 2, ..., 13} in the -A datasets or i E {1, 2, ..., 100} in the -B datasets. In the F6 dataset, the values of the input features of an instance were independently sampled from two uniform distributions: Xᵢ~U(0,1), i ∈ {1, 2, 3, 6, 7, 9, 11, 12, 13} in the -A datasets and i ∈ {1, 2, 3,6,7,9,11,..., 100} in the -B datasets; and Xᵢ~U(0.6,1), i ∈ {4, 5, 8, 10} in both. The value of the target for an instance was computed using the following five functions:

(F6-A) and (F6-B):

$${π}^{{X}_{1} ∗ {X}_{2}} ∗ \sqrt{{X}_{3}} + {sin}^{- 1} ( {X}_{4} ) + log ( {X}_{3} + {X}_{5} ) + \frac{{x}_{9}}{{x}_{10}} * \sqrt{\frac{{x}_{7}}{{x}_{8}}} - {X}_{2} * {X}_{7}$$

. This function contains eleven pairwise feature interactions: {(X₁,X₂), (X₁,X₃), (X₂,X₃), (X₃,X₅), (X₇,X₈), (X₇,X₉), (X₇,X₁₀), (X₈,X₉), (X₈,X₁₀), (X₉,X₁₀), (X₂,X₇)}.

(F7-A) and (F7-B):

$$\begin{matrix}exp \left(\left|{X}_{1} - {X}_{2}\right|\right) + \left|{X}_{2} * {X}_{3}\right| - {X}_{3}^{2 \left|{X}_{4}\right|} + log \left({X}_{4}^{2} + {X}_{5}^{2} + {X}_{7}^{2} + {X}_{8}^{2}\right) \\ + {X}_{9} + {X}_{10}^{2} \frac{1}{1 + {X}_{10}^{2}}\end{matrix}$$

This function contains nine pairwise interactions: {(X₁X₂₎, (X₂,X₃), (X₃,X₄), (X₄,X₅), (X₄,X₇), (X₄,X₈), (X₅,X₇), (X₅,X₈), (X_{7,}X₈)}.

(F8-A) and (F8-B):

$$\begin{matrix}sin \left(\left|{X}_{1} * {X}_{2}\right| + 1\right) - log \left(\left|{X}_{3} * {X}_{4}\right| + 1\right) + cos \left({X}_{5} + {X}_{6} - {X}_{8}\right) \\ + \sqrt{{X}_{8} {}^{2} + {X}_{9} {}^{2} + {X}_{10} {}^{2}} .\end{matrix}$$

This function contains ten pairwise interactions: { (X₁,X₂), (X₃,X₄), (X₅,X₆), (X₄,X₇), (X₅,X₆), (X₅,X₈), (X₆,X₈), (X₈,X₉), (X₈,X₁₀), (X₉,X₁₀)}.

(F9-A) and (F9-B):

$$\begin{matrix}tanh \left({X}_{1} * {X}_{2} + {X}_{3} * {X}_{4}\right) * \sqrt{\left|{X}_{5}\right|} + log \left[{\left({X}_{6} * {X}_{7} * {X}_{8}\right)}^{2} + 1\right] \\ + {X}_{9} * {X}_{10} + \frac{1}{1 + \left|{X}_{10}\right|} .\end{matrix}$$

This function contains thirteen pairwise interactions: {(X₁,X₂), (X₁,X₃), (X₂,X₃), (X₂,X₄), (X₃,X₄), (X₁,X₅), (X₂,X₅), (X₃,X₅), (X₄,X₅), (X₆,X₇), (X₆,X₈), (X₇,X₈), (X₉,X₁₀)}.

(F10-A) and (F10-B): cos(X₁ * X₂ * X₃) + sin(X₄ * X₅ * X₆). This function contains six pairwise interactions: {(X₁,X₂), (X₁,X₃), (X₂,X₃), (X₄,X₅), (X_{4,}X₆), (X₅,X₆)}.

(D) Breast cancer dataset. The Discovery, Biology, and Risk of Inherited Variants in Breast Cancer (DRIVE) project (Amos et al., 2017) generated a breast cancer dataset (NIH dbGaP accession number: phs001265.v1.p1) for genome-wide association study (GWAS) and predictive genomics. This cohort contained 26,053 case subjects with malignant tumor or in situ tumor and 23,058 control subjects with no tumor. The task for predictive genomics is a binary classification of subjects between cases and controls. The breast cancer dataset was processed using PLINK (Purcell et al., 2007), as described in Examples 1-4, to compute the statistical significance of the SNPs. Out of a total of 528,620 SNPs, 1541 SNPs had a p-value lower than 10⁻⁶ and were used as the input features for predictive genomics. To benchmark the performance of the model interpretation, 1541 decoy SNPs were added as input features. The frequencies of homozygous minor alleles, heterozygous alleles, and homozygous dominant alleles were the same between decoy SNPs and real SNPs. Because decoy SNPs have random relationships with the case/control phenotype, they should not be selected as important features or be included in salient interactions by model interpretation.

(E) Implementations and evaluation strategies. The California Housing Dataset was partitioned into a training set (70%), a validation set (20%), and a test set (10%). The eight input features were longitude, latitude, median age, total rooms, total bedrooms, population, households, and median income. The median house value was the target of the regression. All the input features were standardized to zero mean and unit standard deviation based on the training set. Feature standardization is critical for model interpretation in this case because the scale for the importance scores of a feature is determined by the scale for the values of this feature and comparison of the importance scores between features requires the values of the features to be in the same scale. The LINA model comprised an input layer (8 neurons), five fully connected hidden layers (7, 6, 5, 4 and 3 neurons), and an attention layer (8 neurons) for the inner attention neural network, followed by a second input layer (8 neurons), a linearization layer (8 neurons), and an output layer (1 neuron). The hidden layers used ReLU as the activation function. No regularization was applied to the coefficient vector and L1 regularization was applied to the attention vector (γ = 10⁻⁶). The LINA model was trained using the Adam optimizer with a learning rate of 10⁻². The predictive performance of the obtained LINA model was benchmarked to have an RMSE of 71055 in the test set. As a baseline model for comparison, a gradient boosting model achieved an RMSE of 77852 in the test set using 300 decision trees with a maximum depth of 5.

For the first-order interpretation, each synthetic dataset was split into a cross-validation set (80%) for model training and hyperparameter optimization and a test set (20%) for performance benchmarking and model interpretation. A LINA model and a feedforward neural network (FNN) model were constructed using 10-fold cross-validation. For the first four synthetic datasets, the inner attention neural network in the LINA model had 3 layers containing 9 neurons in the first layer, 5 neurons in the second layer, and 10 neurons in the attention layer. The FNN had 3 hidden layers with the same number of neurons in each layer as the inner attention neural network in the LINA model. For the fifth function with more complex relationships, the first and second layers were widened to 100 and 25 neurons, respectively, in both the FNN and LINA models to achieve a predictive performance similar to the other datasets in their respective validation sets. Both the FNN and LINA models were trained using the Adam optimizer. The learning rate was set to 10⁻². The mini-batch size was set to 32. No hyperparameter tuning was performed. The LINA model was trained with the L2 regularization on the coefficient vector (β = 10⁻⁴) and the L1 regularization on the attention vector (γ = 10⁻⁶). The values of β and γ were selected from 10⁻², 10⁻³, 10⁻⁴, 10⁻⁵, 10⁻⁶, 10⁻⁷, and 0 based on the predictive performance of the LINA model on the validation set. Batch normalization was used for both architectures. Both the FNN and LINA models achieved predictive performance at approximately 99% AUC on the test set in the five first-order synthetic datasets, which was comparable to Chen et al. (2018). Deep Lift (Shrikumar et al., 2017), LIME (Ribeiro et al., 2016), Grad*Input (Shrikumar et al., 2017), L2X (Dandl et al., 2020) and Saliency (Simonyan et al., 2014) were used to interpret the FNN model and calculate the feature importance scores using their default configurations. FP, DP, and IP scores were used as the first-order importance scores for the LINA model. The inventors compared the performances of the first-order interpretation of LINA with DeepLIFT, LIME, Grad*Input and L2X. The interpretation accuracy was measured using the Spearman rank correlation coefficient between the predicted ranking of features by their first-order importance and the ground-truth ranking. This metric was chosen because it encompasses both the selection and ranking of the important features.

For the second-order interpretation benchmarking, each synthetic dataset was also split into a cross-validation set (80%) and a test set (20%). A LINA model, an FNN model for NID, and a Bayesian neural network (BNN) for GEH as shown in Cui et al. (2020), were constructed based on the neural network architecture used in (Tsang et al., 2018) using 10-fold cross-validation. The inner attention neural network in the LINA model uses 140 neurons in the first hidden layer, 100 neurons in the second hidden layer, 60 neurons in the third hidden layer, 20 neurons in the fourth hidden layer, and 13 neurons in the attention layer. The FNN model was composed of 4 hidden layers with the same number of neurons in each layer as LINA’s inner attention neural network. The BNN model uses the same architecture as that of the FNN model. The FNN, BNN and LINA models were trained using the Adam optimizer with a learning rate of 10⁻³ and a mini-batch size of 32 for the -A datasets and 128 for the -B datasets. The LINA model was trained using L2 regularization on the coefficient vector (β = 10⁻⁴) and the L1 regularization on the attention vector (γ = 10⁻⁶) with batch normalization. Hyperparameter tuning was performed as described above to optimize the predictive performance. The FNN and BNN models were trained using the default regularization parameters, as shown in Cui et al. (2020) and Tsang et al. (2018). Batch normalization was used for LINA. The FNN, BNN and LINA models all achieved R² scores of more than 0.99 on the test sets of the five -A datasets, as in the examples in Tsang et al. (2018), while their R² scores ranged from 0.91 to 0.93 on the test set of the five high-dimensional -B datasets. Pairwise interactions in each dataset were identified from the BNN model using GEH (Cui et al., 2020), the FNN model using NID (Tsang et al., 2018), and the LINA model using the SP scores. For GEH, the number of clusters was set to the number of features and the number of iterations was set to 20. NID was run using its default configuration. For a dataset with m pairs of ground-truth interactions, the top-m pairs with the highest interaction scores were selected from each algorithm’s interpretation output. The percentage of ground-truth interactions in the top-m predicted interactions (i.e., the precision) was used to benchmark the second-order interpretation performance of the algorithms.

For the breast cancer dataset, 49,111 subjects in the breast cancer dataset were randomly divided into the training set (80%), validation set (10%), and test set (10%). The FNN model and the BNN model had 3 hidden layers with 1000, 250 and 50 neurons as described in Examples 1-4. The same hyperparameters were used in Examples 1-4. The inner attention neural network in the LINA model also used 1000, 250 and 50 neurons before the attention layer. All of these models had 3082 input neurons for 1541 real SNPs and 1541 decoy SNPs. β was set to 0.01 and γ to 0, which were selected from 10⁻², 10⁻³, 10⁻⁴, 10⁻⁵, 10⁻⁶, 10⁻⁷, and 0 based on the predictive performance of the LINA model on the validation set. Early stopping based on the validation AUC score was used during training. The FNN, BNN and LINA models achieved a test AUC of 64.8%, 64.8% and 64.7% on the test set, respectively, using both the 1541 real SNPs with p-values less than 10⁻⁶ and the 1541 decoy SNPs. The test AUCs of these models were lower than that of the FNN model in Examples 1-4 at 67.4% using real 5,273 SNPs with p-values less than 10⁻³ as input. As the same FNN architecture design was used in the two studies, the reduction in the predictive performance in this study can be attributed to the use of more stringent p-value filtering to retain only real SNPs with a high likelihood of having a true association with the disease and the addition of decoy SNPs for benchmarking the interpretation performance.

Deep Lift (Shrikumar et al., 2017), LIME (Ribeiro et al., 2016), Grad*Input (Shrikumar et al., 2017), L2X (Chen et al., 2018) and Saliency (Simonyan et al., 2014) were used to interpret the FNN model and calculate the feature importance scores using their default configurations. The FP score was used as the first-order importance score for the LINA model. After the SNPs were filtered at a given importance score threshold, the false discovery rate (FDR) was computed from the retained real and decoy SNPs above the threshold. The number of retained real SNPs was the total positive count for the FDR. The number of false positive hits (i.e., the number of unimportant real SNPs) within the retained real SNPs was estimated as the number of retained decoy SNPs. Thus, FDR was estimated by dividing the number of retained decoy SNPs by the number of retained real SNPs. An importance-score-sorted list of SNPs from each algorithm was filtered at an increasingly stringent score threshold until reaching the desired FDR level. The interpretation performance of an algorithm was measured by the number of top-ranked features filtered at 0.1%, 1% and 5% FDR and the FDRs for the top-100 and top-200 SNPs ranked by an algorithm.

For the second-order interpretation, pairwise interactions were identified from the BNN model using GEH (Cui et al., 2020), from the FNN model using NID (Tsang et al., 2018), and from the LINA model using the SP scores. For GEH, the number of clusters was set to 20 and the number of iterations was set to 20. While LINA and NID used all 4,911 subjects in the test set and completed their computation within an hour, the GEH results were computed for only 1000 random subjects in the test set over >2 days because GEH would have taken approximately two months to complete the entire test set with its n² computing cost where n is the number of subjects. NID was run using its default configuration in the FNN model. The interpretation accuracy was also measured by the numbers of top-ranked pairwise interactions detected at 0.1%, 1% and 5% FDR and the FDRs for the top-1000 and top-2000 interaction pairs ranked by an algorithm. A SNP pair was considered to be false positive if one or both of the SNPs in a pair was a decoy.

### Results and Discussion

(A) Demonstration of LINA on a real-world application. In this section, the inventors demonstrate LINA using the California housing dataset, which has been used in previous model interpretation studies for algorithm demonstration in Cui et al. (2020) and Tsang et al. (2018). Four types of interpretations from LINA were presented, including the instance-wise first-order interpretation, the instance-wise second-order interpretation, the model-wise first-order interpretation, and the model-wise second-order interpretation.

1) Instance-wise interpretation. Table 9 shows the prediction and interpretation results of the LINA model for an instance (district # 20444) that had a true median price of $208600. The predicted price of $285183 was simply the sum of the eight element-wise products of the attention, coefficient, and feature columns plus the bias. This provided an easily understandable representation of the intermediate computation behind the prediction for this instance. For example, the median age feature had a coefficient of 213 in the model. For this instance, the median age feature had an attention weight of -275, which switched the median age to a negative feature and amplified its direct effect on the predicted price in this district.

The product of the attention weight and coefficient yielded the direct importance score of the median age feature (i.e., DQ = -58,524), which represented the strength of the local linear association between the median age feature and the predicted price for this instance. By assuming that the attention weights of this instance are fixed, one can expect a decrease of $58,524 in the predicted price for an increase in the median age by one standard deviation (12.28 years) for this district. But this did not consider the effects of the median age increase on the attention weights, which was accounted for by its indirect importance score (i.e., IQ = 91,930). The positive IQ indicated that a higher median age would increase the attention weights of other positive features and increase the predicted price indirectly. Combining the DQ and IQ, the positive FQ of 33,407 marked the median age to be a significant positive feature for the predicted price, perhaps through the correlation with some desirable variables for this district. This example suggested a limitation of using the attention weights themselves to evaluate the importance of features in the attentional architectures. The full importance scores represented the total effect of a feature’s change on the predicted price. For this instance, the latitude feature had the largest impact on the predicted price.

Table 10 presents a second-order interpretation of the prediction for this instance. The median age row in Table 10 shows how the median age feature impacted the attention weights of the other features. The two large positive SQ values of median age to the latitude and longitude features indicated significant increases of the two location features’ attention weights with the increase of the median age. In other words, the location become a more important determinant of the predicted price for districts with older houses. The total bedroom feature received a large positive attention weight for this instance. The total bedroom column in Table 10 shows that the longitude and latitude features are the two most important determinants for the attention weights of the total bedroom feature. This suggested how a location change may alter the direct importance of the total bedroom feature for the price prediction of this district.

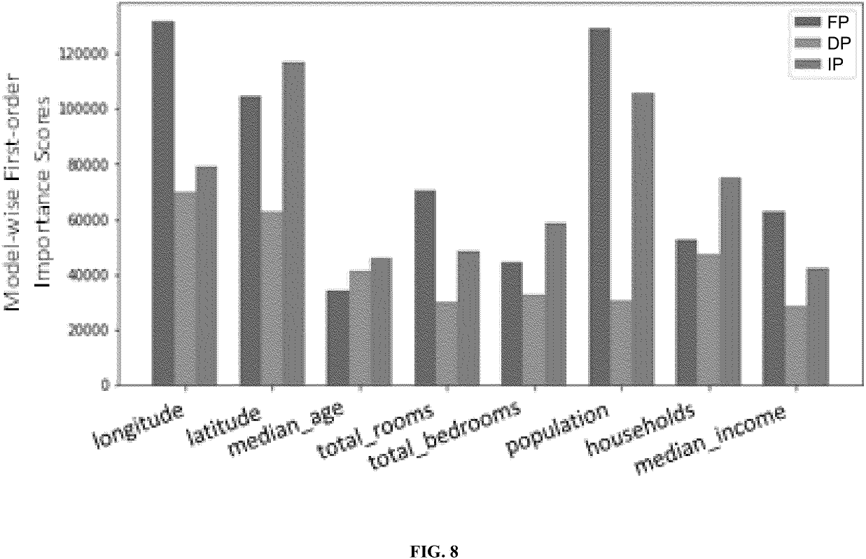

2) Model-wise interpretation. FIG. 8 shows the first-order model-wise interpretation results across districts in the California Housing dataset. The longitude, latitude and population were the three most important features. The longitude and latitude had both high direct importance scores and high indirect importance scores. However, the population feature derived its importance mostly from its heavy influence on the attention weights as measured by its indirect importance score.

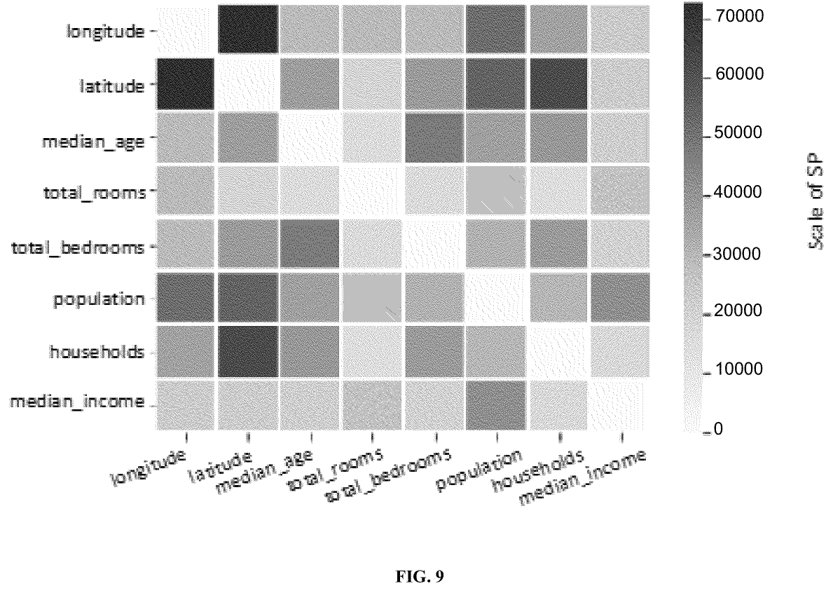

FIG. 9 shows the second-order model-wise interpretation results for pairs of different features. Among all the feature pairs, the latitude and longitude features had the most prominent interactions, which was reasonable because the location was jointly determined by these two features.

Some significant differences existed between the instance-wiseinterpretation and model-wise interpretation (e.g., Table 9 vs. FIG. 8 and Table 10 vs. FIG. 9). This illustrates the need for both instance-wise and model-wise interpretation methods for different purposes.

TABLE 9

The linearization outputs and first-order instance-wise importance scores for a district from the California housing dataset.

| Outputs Features | Linearization Output | | | | First-order Instance-wise Importance Scores | | |
|---|---|---|---|---|---|---|---|
| | Coefficients (K) | Attention (A) | Features (X) | Products (KAX) | FQ | DQ | IQ |
| longitude | 249 | 221 | 0.22 | 11,932 | -51,296 | 55,100 | -106,404 |
| latitude | 257 | -299 | -0.56 | 42,700 | -211,275 | -76,933 | -134,343 |
| median_age | 213 | -275 | -1.35 | 79,230 | 33,407 | -58,524 | 91,930 |
| total_rooms | 173 | 158 | 1.32 | 36,024 | -17,957 | 27,230 | -45,187 |
| total_bedrooms | 184 | 240 | 1.10 | 48,531 | 5,614 | 44,281 | -38,667 |
| population | 200 | -19 | 1.54 | -5,690 | -62,220 | -3,695 | -58,525 |
| households | 189 | 233 | 1.20 | 52,532 | 32,443 | 43,951 | -11,508 |
| median_income | 174 | 125 | 0.91 | 19,777 | 73.337 | 21,736 | 51,601 |
| bias | | | | 149 | | | |
| median_house_price | | | | 285,183 | | | |

TABLE 10

Second-order instance-wise importance scores of feature r (row r) to feature c (column

| Column features (c) Row features (r) | longitude | latitude | median_ age | total_ rooms | total_ bedrooms | population | househoₗds | median_ income |
|---|---|---|---|---|---|---|---|---|
| longitude | -17,234 | -33,983 | 19,682 | -10,797 | 9,572 | -13,375 | -1,153 | 4,899 |
| latitude | 22,696 | 44,572 | 25,631 | 13,068 | 12,002 | 18,119 | 1,035 | -10,005 |
| median_age | 18,591 | 18,555 | -14,252 | 7,140 | 5,749 | 8,326 | 2,586 | -8,357 |
| total_rooms | -13,249 | -27,930 | 11,547 | -4,102 | -4,198 | -8,626 | -526 | 12,029 |
| total_bedrooms | -16,973 | -19,799 | 14,110 | -7,173 | -5,943 | -5,597 | -2,123 | 7,328 |
| population | 932 | 11,223 | -4,307 | 1,052 | 1,947 | 4,842 | -1,471 | -4,623 |
| households | -18,646 | -25,227 | 16,879 | -8,943 | -7,507 | -10,514 | -2,163 | 6,829 |
| median_income | -8,713 | -20,704 | 10,629 | -3,928 | -4,515 | -9,182 | 758 | 9,546 |

(B) Benchmarking of the first-order and second-order interpretations using synthetic datasets. In real-world applications, the true importance of features for prediction cannot be determined with certainty and may vary among different models. Therefore, previous studies on model interpretation (Ribeiro et al., 2016; Cui et al., 2020) benchmarked their interpretation performance using synthetic datasets with known ground-truth of feature importance. In this study, the inventors also compared the interpretation performance of LINA with the SOTA methods using synthetic datasets created as in previous studies (Chen et al., 2021; Tsang et al., 2018).

The performance of the first-order interpretation of LINA was compared with DeepLIFT, LIME, Grad*Input and L2X (Table 11). The three first-order importance scores from LINA, including FP, DP and IP, were tested. The DP score performed the worst among the three, especially in the F3 and F4 datasets which contained interactions among three features. This suggested the limitation of using attention weights as a measure of feature importance. The FP score provided the most accurate ranking among the three LINA scores because it accounted for the direct contribution of a feature and its indirect contribution through attention weights. The first-order importance scores were then compared among different algorithms. L2X and LIME distinguished many important features correctly from un-informative features, but their rankings of the important features were often inaccurate. The gradient-based methods produced mostly accurate rankings of the features based on their first-order importance. Their interpretation accuracy generally decreased in datasets containing interactions among more features. Among all the methods, the LINA FP scores provided the most accurate ranking of the features on average.

The performance of the second-order interpretation of LINA was compared with those of GEH and NID (Table 12). There were a total of 78 possible pairs of interactions among 13 features in each -A synthetic dataset and there were 4950 possible pairs of interactions among 100 features in each -B synthetic dataset. The precision from random guesses was only ∼12.8% on average in the -A datasets and less than 1% in the -B datasets. The three second-order algorithms all performed significantly better than the random guess. In the -A datasets, the average precision of LINA SP was ∼80%, which was ∼12% higher than that of NID and ∼29% higher than that of GEH. The addition of 87 un-informative features in the -B datasets reduced the average precision of LINA by ∼15%, that of NID by ∼13%, and that of GEH by ∼22%. In the -B datasets, the average precision of LINA SP was ∼65%, which was ∼9% higher than that of NID and ∼35% higher than that of GEH. This indicates that more accurate second-order interpretations can be obtained from the LINA models.

TABLE 11

Benchmarking of the first-order interpretation performance using five synthetic datasets (F1∼F5)*

| Datasets | F1 | F2 | F3 | F4 | F5 | Average |
|---|---|---|---|---|---|---|
| Methods | | | | | | |
| LINA DP | 1.00±0.00 | 0.88±0.03 | 0.25±0.07 | 0.65±0.05 | 0.92±0.03 | 0.74±0.04 |
| LINA IP | 1.00±0.00 | 0.92±0.03 | 0.69±0.01 | 0.84±0.03 | 0.96±0.03 | 0.88±0.02 |
| LINA FP | 1.00+0.00 | 0.97±0.02 | 1.00±0.00 | 0.91±0.04 | 1.00±0.00 | 0.98±0.01 |
| DeepLift | 0.99±0.01 | 1.00±0.00 | 0.95±0.03 | 0.83±0.12 | 1.00±0.00 | 0.95±0.03 |
| Saliency | 1.00±0.00 | 0.90±0.01 | 1.00±0.00 | 0.76±0.11 | 1.00±0.00 | 0.93±0.03 |
| Grad*lnput | 1.00±0.00 | 1.00±0.00 | 0.85±0.08 | 0.78±0.12 | 1.00±0.00 | 0.93±0.04 |
| L2X | 0.59±0.06 | 0.41±0.07 | 0.15±0.11 | 0.30±0.08 | 0.5±0.03 | 0.39±0.07 |
| LIME | -0.72±0.0 | -0.52±0.08 | -0.14±0.07 | -0.57±0.05 | -0.3±0.06 | -0.45±0.05 |

*The best Spearman correlation coefficient for each synthetic dataset is highlighted in bold

TABLE 12

Precision of the second-order interpretation by LINA SP, NID and GEH in ten synthetic datasets (F6∼F10)*

| Total Features | Datasets | NID | GEH | LINA SP |
|---|---|---|---|---|
| 13 features | F6-A | 44.5%±0.2% | 50.0%±0.2% | 61.8%±0.2% |
| | F7-A | 98.0%±0.1% | 41.0%±0.2% | 92.0%±0.1% |
| | F8-A | 80.6%±0.2% | 48.8%±0.4% | 85.0%±0.2% |
| | F9-A | 62.2%±0.4% | 41.4%±0.3% | 70.0±0.3% |
| | F10-A | 56.7%±0.3% | 75.0%±0.5% | 91.7%±0.3% |
| | Average | 68.4%±0.2% | 51.2%±0.3% | 80.1±0.2% |
| 100 features | F6-B | 51.8%±0.2% | 18.1%±1.0% | 52.7%±0.3% |
| | F7-B | 44.0%±0.2% | 28.8%±0.4% | 90.0%±0.0% |
| | F8-B | 76.3%±0.1% | 47.9%±0.2% | 80%0.0±0.3% |
| | F9-B | 40.0%±0.3% | 41.8%±0.2% | 51.7%±0.3% |
| | F10-B | 66.6%±0.0% | 10.4%±1.0% | 50.0%±0.1% |
| | Average | 55.7%±0.2% | 29.4%±0.6% | 64.9%±0.2% |

*The best precision for each dataset is highlighted in bold

(C) Benchmarking of the first-order and second-order interpretation using a predictive genomics application. As the performance benchmarks in synthetic datasets may not reflect those in real-world applications, the inventors engineered a real-world benchmark based on a breast cancer dataset for predictive genomics. While it was unknown which SNPs and which SNP interactions were truly important for phenotype prediction, the decoy SNPs added by the inventors were truly unimportant. Moreover, a decoy SNP cannot have a true interaction, such as XOR or multiplication, with a real SNP to have a joint impact on the disease outcome. Thus, if a decoy SNP or an interaction with a decoy SNP is ranked by an algorithm as important, it should be considered a false positive detection. As the number of decoy SNPs was the same as the number of real SNPs, the false discovery rate can be estimated by assuming that an algorithm makes as many false positive detections from the decoy SNPs as from the real SNPs. This allowed the inventors to compare the number of positive detections by an algorithm at certain FDR levels.

The first-order interpretation performance of LINA was compared with those of DeepLIFT, LIME, Grad*Input and L2X (Table 13). At 0.1%, 1%, and 5% FDR, LINA identified more important SNPs than other algorithms. LINA also had the lowest FDRs for the top-100 and top-200 SNPs. The second-order interpretation performance of LINA was compared with those of NID and GEH (Table 14). At 0.1%, 1%, and 5% FDR, LINA identified more pairs of important SNP interactions than NID and GEH did. LINA had lower FDRs than the other algorithms for the top-1000 and top-2000 SNP pairs. Both L2X and GEH failed to output meaningful importance scores in this predictive genomics dataset. Because GEH needed to compute the full Hessian, it was also much more computationally expensive than the other algorithms.

The existing model interpretation algorithms and LINA can provide rankings of the features or feature interactions based on their importance scores at arbitrary scales. The inventors demonstrated that decoy features can be used in real-world applications to set thresholds for first-order and second-order importance scores based on the FDRs of retained features and feature pairs. This provided an uncertainty quantification of the model interpretation results without knowing the ground-truth in real-world applications.

The predictive genomics application provided a real-world test of the interpretation performance of these algorithms. In comparison with the synthetic datasets, the predictive genomics dataset was more challenging for model interpretation, because of the low predictive performance of the models and the large number of input features. For this real-world application, LINA was shown to provide better first-order and second-order interpretation performance than existing algorithms on a model-wise level. Furthermore, LINA can provide instance-wise interpretation to identify important SNP and SNP interactions for the prediction of individual subjects. Model interpretation is important for making biological discoveries from predictive models, because first-order interpretation can identify individual genes involved in a disease (Rivandi et al., 2018; Romualdo Cardoso et al., 2021) and second-order interpretation can uncover epistatic interactions among genes for a disease (Shaker & Senousy, 2019; van de Haar et al., 2019). These discoveries may provide new drug targets (Wang et al., 2018; Gao et al., 2019; Gonçalves et al., 2020) and enable personalized formulation of treatment plans (We et al., 2015; Zhao et al., 2021; Velasco-Ruiz et al., 2021) for breast cancer.

TABLE 13

Performance benchmarking of the first-order interpretation for predictive genomics

| Methods | LINA FP | Saliency | grad*Input | DeepLift | LIME | L2X |
|---|---|---|---|---|---|---|
| # SNPs at 0.1% FDR | 127 | 35 | 75 | 75 | 9 | 0 |
| # SNPs at 1% FDR | 158 | 35 | 88 | 85 | 9 | 0 |
| # SNPs at 5% FDR | 255 | 57 | 122 | 119 | 9 | 0 |
| FDR at top-100 SNP | 0.0% | 7.5% | 3.0% | 2.0% | 16.3% | N/A |
| FDR at top-200 SNP | 1.5% | 16.2% | 9.3% | 9.3% | 20.5% | N/A |

TABLE 14

Performance benchmarking of the second-order interpretation for predictive genomics

| Methods | LINA SP | NID | GEH |
|---|---|---|---|
| # SNP pairs at 0.1% FDR | 583 | 415 | 0 |
| # SNP pairs at 1% FDR | 1040 | 504 | 0 |
| # SNP pairs at 5% FDR | 2887 | 810 | 0 |
| FDR at top-1000 SNP pairs | 0.9% | 10.5% | N/A |
| FDR at top-2000 SNP pairs | 3.0% | 31.8% | N/A |

Conclusion. In this study, the inventors designed a new neural network architecture, referred to as LINA, for model interpretation. LINA uses a linearization layer on top of a deep inner attention neural network to generate a linear representation of model prediction. LINA provides the unique capability of offering both first-order and second-order interpretations and both instance-wise and model-wise interpretations. The interpretation performance of LINA was benchmarked to be higher than the existing algorithms on synthetic datasets and a predictive genomics dataset.

While the compositions, apparatus, and methods of this disclosure have been described in terms of particular embodiments, it will be apparent to those of skill in the art that variations may be applied to the methods and in the steps or in the sequence of steps of the method described herein without departing from the concept, spirit and scope of the disclosure. All such similar variations and modifications apparent to those skilled in the art are deemed to be within the spirit, scope and concept of the inventive concepts as defined by the appended claims.

### REFERENCES

The following references, to the extent that they provide exemplary procedural or other details supplementary to those set forth herein, are specifically incorporated herein by reference in their entireties.

Amos et al., “The OncoArray Consortium: A Network for Understanding the Genetic Architecture of Common Cancers,” Cancer Epidemiol Biomarkers Prev , vol. 26, no. 1, pp. 126-135, January 2017, doi: 10.1158/1055-9965.EPI-16-0106.

Amos et al., “The OncoArray Consortium: A Network for Understanding the Genetic Architecture of Common Cancers,” Cancer Epidemiol Biomarkers Prev, vol. 26, no. 1, pp. 126-135, January 2017, doi: 10.1158/1055-9965.EPI-16-0106.

Angemueller, T. Pärnamaa, L. Parts, and O. Stegle, “Deep learning for computational biology,” Molecular Systems Biology, vol. 12, no. 7, p. 878, July 2016, doi: 10.15252/msb.20156651.

Badre, L. Zhang, W. Muchero, J. C. Reynolds, and C. Pan, “Deep neural network improves the estimation of polygenic risk scores for breast cancer,” Journal of Human Genetics, pp. 1-11, October 2020, doi: 10.1038/s10038-020-00832-7.

Baltres et al., “Prediction of Oncotype DX recurrence score using deep multi-layer perceptrons in estrogen receptor-positive, HER2-negative breast cancer,” Breast Cancer, vol. 27, no. 5, pp. 1007-1016, September 2020, doi: 10.1007/s12282-020-01100-4.

Bellot, G. de los Campos, and M. Pérez-Enciso, “Can Deep Learning Improve Genomic Prediction of Complex Human Traits?,” Genetics, vol. 210, no. 3, pp. 809-819, November 2018, doi: 10.1534/genetics.118.301298.

Bengio, “Learning Deep Architectures for AI,” Found. Trends Mach. Learn., vol. 2, no. 1, pp. 1-127, January 2009, doi: 10.1561/2200000006.

Bermeitinger, T. Hrycej, and S. Handschuh, “Representational Capacity of Deep Neural Networks — A Computing Study,” Proceedings of the 11th International Joint Conference on Knowledge Discovery, Knowledge Engineering and Knowledge Management, pp. 532-538, 2019, doi: 10.5220/0008364305320538.

Cesaratto et al., “BNC2 is a putative tumor suppressor gene in high-grade serous ovarian carcinoma and impacts cell survival after oxidative stress,” Cell Death & Disease, vol. 7, no. 9, Art. no. 9, September 2016, doi: 10.1038/cddis.2016.278.

Chan et al., “Evaluation of three polygenic risk score models for the prediction of breast cancer risk in Singapore Chinese,” Oncotarget, vol. 9, no. 16, pp. 12796-12804, January 2018, doi: 10.18632/oncotarget.24374.

Chang, C. C. Chow, L. C. Tellier, S. Vattikuti, S. M. Purcell, and J. J. Lee, “Second-generation PLINK: rising to the challenge of larger and richer datasets,” Gigascience, vol. 4, no. 1, December 2015, doi: 10.1186/s13742-015-0047-8.

Chen, L. Song, M. Wainwright, and M. Jordan, “Learning to Explain: An Information-Theoretic Perspective on Model Interpretation,” in Proceedings of the 35th International Conference on Machine Learning, July 2018, pp. 883-82. Accessed: Nov. 04, 2021. [Online]. Available: https://proceedings.mlr.press/v80/chen18j.html

Clark, B. P. Kinghorn, J. M. Hickey, and J. H. van der Werf, “The effect of genomic information on optimal contribution selection in livestock breeding programs,” Genetics Selection Evolution, vol. 45, no. 1, p. 44, October 2013, doi: 10.1186/1297-9686-45-44.

Cordell, “Epistasis: what it means, what it doesn’t mean, and statistical methods to detect it in humans,” Human Molecular Genetics, vol. 11, no. 20, pp. 2463-2468, October 2002, doi: 10.1093/hmg/11.20.2463.

Cudic, H. Baweja, T. Parhar, and S. Nuske, “Prediction of Sorghum Bicolor Genotype from In-Situ Images Using Autoencoder-Identified SNPs,” in 2018 17th IEEE International Conference on Machine Learning and Applications (ICMLA), December 2018, pp. 23-31, doi: 10.1109/ICMLA.2018.00012.

Cui, P. Marttinen, and S. Kaski, “Learning Global Pairwise Interactions with Bayesian Neural Networks,” in ECAI 2020 — 24th European Conference on Artificial Intelligence, 29 August-8 September 2020, Santiago de Compostela, Spain, August 29 - September 8, 2020 - Including 10th Conference on Prestigious Applications of Artificial Intelligence (PAIS 2020), 2020, vol. 325, pp. 1087-1094. doi: 10.3233/FAIA200205.

Dandl, C. Molnar, M. Binder, and B. Bischl, “Multi-Objective Counterfactual Explanations,” in Parallel Problem Solving from Nature - PPSN XVI, Cham, 2020, pp. 448-469. doi: 10.1007/978-3-030-58112-1_31.

Dayem Ullah, J. Oscanoa, J. Wang, A. Nagano, N. R. Lemoine, and C. Chelala, “SNPnexus: assessing the functional relevance of genetic variation to facilitate the promise of precision medicine,” Nucleic Acids Res, vol. 46, no. W1, pp. W109-W113, July 2018, doi: 10.1093/nar/gky399.

De, W. S. Bush, and J. H. Moore, “Bioinformatics Challenges in Genome-Wide Association Studies (GWAS),” in Clinical Bioinformatics, R. Trent, Ed. New York, NY: Springer, 2014, pp. 63-81.

Do and N. Q. K. Le, “Using extreme gradient boosting to identify origin of replication in Saccharomyces cerevisiae via hybrid features,” Genomics, vol. 112, no. 3, pp. 2445-2451, mai 2020, doi: 10.1016/j.ygeno.2020.01.017.

Dudbridge, “Power and Predictive Accuracy of Polygenic Risk Scores,” PLOS Genetics, vol. 9, no. 3, p. e1003348, March 2013, doi: 10.1371/journal.pgen.1003348.

Fergus, A. Montanez, B. Abdulaimma, P. Lisboa, C. Chalmers, and B. Pineles, “Utilising Deep Learning and Genome Wide Association Studies for Epistatic-Driven Preterm Birth Classification in African-American Women,” IEEE/ACM Transactions on Computational Biology and Bioinformatics, pp. 1-1, 2018, doi: 10.1109/TCBB.2018.2868667.

Fernald and M. Kurokawa, “Evading apoptosis in cancer,” Trends in Cell Biology, vol. 23, no. 12, pp. 620-633, December 2013, doi: 10.1016/j.tcb.2013.07.006.

Friedman, “Greedy function approximation: a gradient boosting machine,” Annals of statistics, pp. 1189-1232, 2001.

Gao, Y. Quan, X.-H. Zhou, and H.- Y. Zhang, “PheW AS-Based Systems Genetics Methods for Anti-Breast Cancer Drug Discovery,” Genes, vol. 10, no. 2, Art. no. 2, February 2019, doi: 10.3390/genes10020154.

Ge, C.-Y. Chen, Y. Ni, Y.-C. A. Feng, and J. W. Smoller, “Polygenic prediction via Bayesian regression and continuous shrinkage priors,” Nature Communications, vol. 10, no. 1, pp. 1-10, April 2019, doi: 10.1038/s41467-019-09718-5,

Gola, J. Erdmann, B. Müller-Myhsok, H. Schunkert, and I. R. König, “Polygenic risk scores outperform machine learning methods in predicting coronary artery disease status,” Genetic Epidemiology, vol. 44, no. 2, pp. 125-138, March 2020, doi: 10.1002/gepi.22279.

Goncalves et al., “Drug mechanism-of-action discovery through the integration of pharmacological and CRISPR screens,” Molecular Systems Biology, vol. 16, no. 7, p. e9405, 2020, doi: https://doi.org/10.15252/msb.20199405.

Hastie, R. Tibshirani, and J. Friedman, The Elements of Statistical Learning: Data Mining, Inference, and Prediction, Second Edition, 2nd ed. New York: Springer-Verlag, 2009.

Hastie, S. Rosset, J. Zhu, and H. Zou, “Multi-class adaboost,” Statistics and its Interface, vol. 2, no. 3, pp. 349-360, 2009.

Ho Thanh Lam et al., “Machine Learning Model for Identifying Antioxidant Proteins Using Features Calculated from Primary Sequences,” Biology, vol. 9, no. 10, Art. no. 10, October 2020, doi: 10.3390/biology9100325.

Ho, W. Schierding, M. Wake, R. Saffery, and J. O’Sullivan, “Machine Learning SNP Based Prediction for Precision Medicine,” Front. Genet., vol. 10, 2019, doi: 10.3389/fgene.2019.00267.

Hsieh et al., “A polygenic risk score for breast cancer risk in a Taiwanese population,” Breast Cancer Res Treat, vol. 163, no. 1, pp. 131-138, May 2017, doi: 10.1007/s10549-017-4144-5.

International Schizophrenia Consortium et al., “Common polygenic variation contributes to risk of schizophrenia and bipolar disorder,” Nature, vol. 460, no. 7256, pp. 748-752, August 2009, doi: 10.1038/nature08185.

Ioffe and C. Szegedy, “Batch Normalization: Accelerating Deep Network Training by Reducing Internal Covariate Shift,” arXiv:1502.03167 [cs], March 2015, Accessed: Nov. 25, 2019. [Online]. Available: http://arxiv.org/abs/1502.03167.

Kelley Pace and R. Barry, “Sparse spatial autoregressions,” Statistics & Probability Letters, vol. 33, no. 3, pp. 291-297, May 1997, doi: 10.1016/S0167-7152(96)00140-X

Khera et al., “Genome-wide polygenic scores for common diseases identify individuals with risk equivalent to monogenic mutations,” Nat Genet, vol. 50, no. 9, pp. 1219-1224, September 2018, doi: 10.1038/s41588-018-0183-z.

Kingma and J. Ba, “Adam: A Method for Stochastic Optimization,” 3rd International Conference for Learning Representations, 2015, Accessed: Nov. 26, 2019. [Online]. Available: http://arxiv.org/abs/1412.6980.

Kolch, M. Halasz, M. Granovskaya, and B. N. Kholodenko, “The dynamic control of signal transduction networks in cancer cells,” Nature Reviews Cancer, vol. 15, no. 9, Art. no. 9, September 2015, doi: 10.1038/nrc3983.

LeBlanc and C. Kooperberg, “Boosting predictions of treatment success,” Proc Natl Acad Sci U SA, vol. 107, no. 31, pp. 13559-13560, August 2010, doi: 10.1073/pnas.1008052107.

Lee et al., “Candidate gene approach evaluates association between innate immunity genes and breast cancer risk in Korean women,” Carcinogenesis, vol. 30, no. 9, pp. 1528-1531, September 2009, doi: 10.1093/carcin/bgp084.

Li et al., “NOS1 upregulates ABCG2 expression contributing to DDP chemoresistance in ovarian cancer cells,” Oncology Letters, vol. 17, no. 2, pp. 1595-1602, February 2019, doi: 10.3892/ol.2018.9787.

Lundberg and S.-I. Lee, “A Unified Approach to Interpreting Model Predictions,” in Advances in Neural Information Processing Systems, 2017, vol. 30. Accessed: Jan. 31, 2022. [Online]. Available: https://papers.nips.cc/paper/2017/hash/8a20a8621978632d76c43dfd28b67767-Abstract.html

Maier et al., “Joint analysis of psychiatric disorders increases accuracy of risk prediction for schizophrenia, bipolar disorder, and major depressive disorder,” Am. J. Hum. Genet., vol. 96, no. 2, pp. 283-294, February 2015, doi: 10.1016/j.ajhg.2014.12.006.

Mao and J. D. Unadkat, “Role of the Breast Cancer Resistance Protein (BCRP/ABCG2) in Drug Transport—an Update,” AAPS J, vol. 17, no. 1, pp. 65-82, January 2015, doi: 10.1208/s12248-014-9668-6.

Mavaddat et al., “Polygenic Risk Scores for Prediction of Breast Cancer and Breast Cancer Subtypes,” The American Journal of Human Genetics, vol. 104, no. 1, pp. 21-34, January 2019, doi: 10.1016/j.ajhg.2018.11.002.

Meuwissen, B. J. Hayes, and M. E. Goddard, “Prediction of Total Genetic Value Using Genome-Wide Dense Marker Maps,” Genetics, vol. 157, no. 4, pp. 1819-1829, April 2001.

Michailidou et al., “Association analysis identifies 65 new breast cancer risk loci,” Nature, vol. 551, no. 7678, pp. 92-94, November 2017, doi: 10.1038/nature24284.

Molnar, Interpretable machine learning. A Guide for Making Black Box Models Explainable. 2019. [Online], Available: https://christophm.github.io/interpretable-ml-book/

Nelson, K. Tyne, A. Naik, C. Bougatsos, B. K. Chan, and L. Humphrey, “Screening for Breast Cancer: An Update for the U.S. Preventive Services Task Force,” Annals of Internal Medicine, vol. 151, no. 10, pp. 727-737, November 2009, doi: 10.7326/0003-4819-151-10-200911170-00009.

NIH, “Female Breast Cancer - Cancer Stat Facts.” https://seer.cancer.gov/statfacts/html/breast.html (accessed Dec. 03, 2019).

O’Connor, “Targeting the DNA Damage Response in Cancer,” Molecular Cell, vol. 60, no. 4, pp. 547-560, November 2015, doi: 10.1016/j.molcel.2015.10.040.

Oeffinger et al., “Breast Cancer Screening for Women at Average Risk: 2015 Guideline Update From the American Cancer Society,” JAMA, vol. 314, no. 15, pp. 1599-1614, October 2015, doi: 10.1001/jama.2015.12783.

Phillips, “Epistasis - the essential role of gene interactions in the structure and evolution of genetic systems,” Nat Rev Genet, vol. 9, no. 11, pp. 855-867, November 2008, doi: 10.1038/nrg2452.

Purcell et al., “PLINK: A Tool Set for Whole-Genome Association and Population-Based Linkage Analyses,” The American Journal of Human Genetics, vol. 81, no. 3, pp. 559-575, September 2007, doi: 10.1086/519795.

Ribeiro, S. Singh, and C. Guestrin, “‘Why Should I Trust You?’: Explaining the Predictions of Any Classifier,” in Proceedings of the 22Nd ACM SIGKDD International Conference on Knowledge Discovery and Data Mining, New York, NY, USA, 2016, pp. 1135-1144, doi: 10.1145/2939672.2939778.

Rivandi, J. W. M. Martens, and A. Hollestelle, “Elucidating the Underlying Functional Mechanisms of Breast Cancer Susceptibility Through Post-GWAS Analyses,” Frontiers in Genetics, vol. 9, 2018, Accessed: Feb. 01, 2022. [Online]. Available: frontiersin.org/article/10.3389/fgene.2018.00280

Romualdo Cardoso, A. Gillespie, S. Haider, and O. Fletcher, “Functional annotation of breast cancer risk loci: current progress and future directions,” Br J Cancer, pp. 1-13, November 2021, doi: 10.1038/s41416-021-01612-6.

Schmidhuber, “Deep learning in neural networks: An overview,” Neural Networks, vol. 61, pp. 85-117, January 2015, doi: 10.1016/j.neunet.2014.09.003.

Scott et al., “An Expanded Genome-Wide Association Study of Type 2 Diabetes in Europeans,” Diabetes, May 2017, doi: 10.2337/db16-1253.

Shaker and M. A. Senousy, “Association of SNP-SNP interactions Between RANKL, OPG, CHI3L1, and VDR Genes With Breast Cancer Risk in Egyptian Women,” Clinical Breast Cancer, vol. 19, no. 1, pp. e220-e238, février 2019, doi: 10.1016/j.clbc.2018.09.004.

Shrikumar, P. Greenside, and A. Kundaje, “Learning Important Features Through Propagating Activation Differences,” in International Conference on Machine Learning, July 2017, pp. 3145-3153, Accessed: Nov. 11, 2019. [Online]. Available: http://proceedings.mlr.press/v70/shrikumar17a.html.

Simonyan, A. Vedaldi, and A. Zisserman, “Deep Inside Convolutional Networks: Visualising Image Classification Models and Saliency Maps,” 2014.

Sorokina, R. Caruana, and M. Riedewald, “Additive Groves of Regression Trees,” in Proceedings of the 18th European conference on Machine Learning, Berlin, Heidelberg, September 2007, pp. 323-334. doi: 10.1007/978-3-540-74958-5_31.

Speed and D. J. Balding, “MultiBLUP: improved SNP-based prediction for complex traits,” Genome Res., vol. 24, no. 9, pp. 1550-1557, September 2014, doi: 10.1 101/gr.169375.113.

Srivastava, G. Hinton, A. Krizhevsky, I. Sutskever, and R. Salakhutdinov, “Dropout: A Simple Way to Prevent Neural Networks from Overfitting,” Journal of Machine Learning Research, vol. 15, pp. 1929-1958, 2014.

Tinholt et al., “Increased coagulation activity and genetic polymorphisms in the F5, F10 and EPCR genes are associated with breast cancer: a case-control study,” BMC Cancer, vol. 14, November 2014, doi: 10.1 186/1471-2407-14-845.

Tsang, D. Cheng, and Y. Liu, “Detecting Statistical Interactions from Neural Network Weights,” 2018.

Tsuboi et al., “Prognostic significance of GAD1 overexpression in patients with resected lung adenocarcinoma,” Cancer Medicine, vol. 8, no. 9, pp. 4189-4199, 2019, doi: 10.1002/cam4.2345.

van de Haar, S. Canisius, M. K. Yu, E. E. Voest, L. F. A. Wessels, and T. Ideker, “Identifying Epistasis in Cancer Genomes: A Delicate Affair,” Cell, vol. 177, no. 6, pp. 1375-1383, mai 2019, doi: 10.1016/j.cell.2019.05.005.

Velasco-Ruiz et al., “POLRMT as a Novel Susceptibility Gene for Cardiotoxicity in Epirubicin Treatment of Breast Cancer Patients,” Pharmaceutics, vol. 13, no. 11, Art. no. 11, November 2021, doi: 10.3390/pharmaceutics13111942.

Vilhjálmsson et al., “Modeling Linkage Disequilibrium Increases Accuracy of Polygenic Risk Scores,” Am J Hum Genet, vol. 97, no. 4, pp. 576-592, October 2015, doi: 10.1016/j.ajhg.2015.09.001. Wang, J. Ingle, and R. Weinshilboum, “Pharmacogenomic Discovery to Function and Mechanism: Breast Cancer as a Case Study,” Clinical Pharmacology & Therapeutics, vol. 103, no. 2, pp. 243-252, 2018, doi: 10.1002/cpt.915.

Wei et al., “From Disease Association to Risk Assessment: An Optimistic View from Genome-Wide Association Studies on Type 1 Diabetes,” PLOS Genetics, vol. 5, no. 10, p. e1000678, October 2009, doi: 10.1371/journal.pgen.1000678.

Wen et al., “Prediction of breast cancer risk based on common genetic variants in women of East Asian ancestry,” Breast Cancer Research, vol. 18, no. 1, p. 124, December 2016, doi: 10.1186/s13058-016-0786-1.

Whittaker, I. Royzman, and T. L. Orr-Weaver, “Drosophila Double parked: a conserved, essential replication protein that colocalizes with the origin recognition complex and links DNA replication with mitosis and the down-regulation of S phase transcripts,” Genes Dev, vol. 14, no. 14, pp. 1765-1776, July 2000.

Wu et al., “A genome-wide association study identifies WT1 variant with better response to 5-fluorouracil, pirarubicin and cyclophosphamide neoadjuvant chemotherapy in breast cancer patients,” Oncotarget, vol. 7, no. 4, pp. 5042-5052, November 2015, doi: 10.18632/oncotarget.5837.

Xu, N. Wang, T. Chen, and M. Li, “Empirical Evaluation of Rectified Activations in Convolutional Network,” arXiv:1505.00853 [cs, stat], November 2015, Accessed: Nov. 25, 2019. [Online]. Available: http://arxiv.org/abs/1505.00853.

Yin et al., “Using the structure of genome data in the design of deep neural networks for predicting amyotrophic lateral sclerosis from genotype,” bioRxiv, p. 533679, January 2019, doi: 10.1101/533679.

Zhao, J. Li, Z. Liu, and S. Powers, “Combinatorial CRISPR/Cas9 Screening Reveals Epistatic Networks of Interacting Tumor Suppressor Genes and Therapeutic Targets in Human Breast Cancer,” Cancer Res, vol. 81, no. 24, pp. 6090-6105, December 2021, doi: 10.1158/0008-5472.CAN-21-2555.

What is claimed is:

1. A computer-implemented method of training a deep neural network for estimating a polygenic risk score for a disease, the method comprising:
collecting a first set of SNPs from at least 1,000 subjects with a known disease outcome from a database and a second set of SNPs from at least 1,000 other subjects with a known disease outcome from a database,
encoding, independently, the first set of SNPs and the second set of SNPs by:
labeling each subject as either a disease case or a control case based on the known disease outcome for the subject, and
labeled each SNP in each subject as either homozygous with minor allele, heterozygous allele, or homozygous with the dominant allele;
optionally applying one or more filter to the first encoded set to create a first modified set of SNPs;
training the deep neural network using the first encoded set of SNPs or the first modified set of SNPs; and
validating the deep neural network using the second encoded set of SNPs.

2. The method of claim 1, wherein the filter comprises a p-value threshold.

3. The method of claim 1, wherein the first set of SNPs and the second set of SNPs are both from at least 10,000 subjects.

4. The method of claim 1, wherein the SNPs are genome-wide.

5. The method of claim 4, wherein the SNPs are representative of at least 22 chromosomes.

6. The method of claim 1, wherein both the first set of SNPs and the second set of SNPs comprise the same at least 2,000 SNPs.

7. The method of claim 1, wherein the disease is cancer.

8. The method of claim 7, wherein the cancer is breast cancer.

9. The method of claim 8, wherein the SNPs include at least five of the SNPs listed in Table 2.

10. The method of claim 1, wherein the trained deep neural network has an accuracy of at least 60%.

11. The method of claim 1, wherein the trained deep neural network has an AUC of at least 65%.

12. The method of claim 1, wherein the deep neural network comprises at least three hidden layers, wherein each layer comprises multiple neurons.

13. The method of claim 1, wherein the deep neural network comprises a linearization layer on top of a deep inner attention neural network.

14. The method of claim 13, wherein the linearization layer computes an output as an element-wise multiplication product of input features, attention weights, and coefficients.

15. The method of claim 14, wherein the network learns a linear function of an input feature vector, coefficient vector, and attention vector.

16. The method of claim 15, wherein the attention vector is computed from the input feature vector using a multi-layer neural network.

17. The method of claim 16, wherein all hidden layers of the multi-layer neural network use a non-linear activation function, and wherein the attention layer uses a linear activation function.

18. The method of claim 17, wherein the inner attention neural network uses 1000, 250 and 50 neurons before the attention layer.

19. The method of claim 1, wherein training the deep neural network comprises using stochastic gradient descent with regularization, such as dropout.

20. A method of using a deep neural network trained using data from subjects with a disease by the method of claim 1 to estimate a polygenic risk score for a patient for the disease, the method comprising:
collecting a set of SNPs from a subject with an unknown disease outcome,
encoding the set of SNPs by labeled each SNP in the subject as either homozygous with minor allele, heterozygous allele, or homozygous with the dominant allele;
applying the deep neural network to obtain an estimated polygenic risk score for the patient for the disease.

21. The method of claim 20, further comprising performing, or having performed, further screening for the disease if the polygenic risk score indicates that the patient is at risk for the disease.

22. A method for determining a polygenic risk score for a disease for a subject, comprising:
(a) obtaining a plurality of SNPs from genome of the subject;
(b) generating a data input from the plurality of SNPs; and
(c) determining the polygenic risk score for the disease by applying to the data input a deep neural network trained by the method of claim 1.

23. The method of claim 22, further comprising performing, or having performed, further screening for the disease if the polygenic risk score indicates that the patient is at risk for the disease.

24. The method of claim 23, wherein the disease is breast cancer, and wherein the method comprises performing, or having performed, yearly breast MRI and mammogram if the patient’s polygenic risk score is greater than 20%.

25. A polygenic risk score classifier comprising a deep neural network that has been trained according to the method of claim 1.
